(19) United States
(12) Patent Application Publication
(10) Pub. No.: US 2026/0317448 A1
(43) Pub. Date: Oct. 15, 2026

(54) ALKALINE PHOSPHATASE ANTIBODIES AND USES THEREOF

(71) Applicant: Chosen Diagnostics Inc., New Orleans, LA (US)

(72) Inventors: Sunyoung Kim, New Orleans, LA (US); Rebecca Buckley, Metairie, LA (US)

(21) Appl. No.: 19/483,125

(22) PCT Filed: May 10, 2024
(86) PCT No.: PCT/US2024/028889

§ 371 Date: Nov. 10, 2025

Related U.S. Application Data
(60) Provisional application No. 63/465,361, filed on May 10, 2023.

(51) Int. Cl. C07K 16/40

(52) U.S. Cl. CPC C07K 16/40; C12Y 301/03001; C07K 2317/622

(57) ABSTRACT

The invention is directed to antibodies that bind to intestinal alkaline phosphatase (iAP) protein. The antibodies can be used to detect and treat gastrointestinal disease.

This application is an International Application which claims priority from U.S. Provisional Application No. 63/465,361, filed on May 10, 2023, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The application contains a Sequence Listing which has been submitted electronically in ASCII format and is hereby incorporated by reference in its entirety. Said ASCII copy, created on [ ], is named [ ] and is [ ] bytes in size.

### FIELD OF THE INVENTION

This invention is directed to antibodies against intestinal alkaline phosphatase (iAP) and methods of use thereof.

### BACKGROUND

Intestinal alkaline phosphatase (iAP) plays a pivotal role in maintaining gut health and well-being. iAP is a major regulator of gut mucosal permeability and detoxifies the surface lipopolysaccharide (LPS) of harmful bacteria by cleaving inorganic phosphate. iAP is produced by enterocytes in the small intestine and exerts anti-inflammatory properties.

### SUMMARY OF THE INVENTION

The invention provides for intestinal alkaline phosphatase (iAP) antibody compositions and methods of use of the same.

Aspects of the invention are drawn towards an isolated antibody or fragment thereof that binds to human intestinal alkaline phosphatase (hIAP) comprising a VH CDR1 comprising the amino acid sequence of SEQ ID NO: 3, a VH CDR2 comprising the amino acid sequence of SEQ ID NO: 4, a VH CDR3 comprising the amino acid sequence of SEQ ID NO: 5, or sequences at least 80% identical thereto, and/or a VL CDR1 comprising the amino acid sequence of SEQ ID NO: 6, a VL CDR2 comprising the amino acid sequence of SEQ ID NO: 7, and a VL CDR3 comprising the amino acid sequence of SEQ ID NO: 8, or sequences at least 80% identical thereto.

Aspects of the invention are also drawn towards an isolated antibody or fragment thereof that binds to human intestinal alkaline phosphatase (hIAP) comprising a VH CDR1 comprising the amino acid sequence of SEQ ID NO: 22, a VH CDR2 comprising the amino acid sequence of SEQ ID NO: 23 or SEQ ID NO: 31, a VH CDR3 comprising the amino acid sequence of SEQ ID NO: 24, or sequences at least 80% identical thereto, and/or a VL CDR1 comprising the amino acid sequence of SEQ ID NO: 25, a VL CDR2 comprising the amino acid sequence of SEQ ID NO: 26, and a VL CDR3 comprising the amino acid sequence of SEQ ID NO: 27, or sequences at least 80% identical thereto.

Aspects of the invention are also drawn towards an isolated antibody or fragment thereof that binds to human intestinal alkaline phosphatase (hIAP) comprising a heavy chain variable region comprising an amino acid sequence according to SEQ ID NO: 1 or a sequence that is at least 80% identical thereto, and/or a light chain variable region comprising an amino acid sequence according to SEQ ID NO: 2 or a sequence that is at least 80% identical thereto.

Aspects of the invention are further drawn towards an isolated antibody or fragment thereof that binds to human intestinal alkaline phosphatase (hIAP) comprising a heavy chain variable region comprising an amino acid sequence according to SEQ ID NO: 20 or a sequence that is at least 80% identical thereto, and/or a light chain variable region comprising an amino acid sequence according to SEQ ID NO: 21 or a sequence that is at least 80% identical thereto.

In embodiments, the isolated monoclonal antibody or antigen-binding fragment comprises a wildtype Fc or a modified Fc.

In embodiments, the isolated monoclonal antibody or antigen-binding fragment comprises an Fab fragment, a single-chain variable fragment (scFv), or a single-domain antibody.

In embodiments, the isolated monoclonal antibody or antigen-binding fragment further comprises a detectable moiety.

Further, aspects of the invention are drawn towards a pharmaceutical composition comprising the isolated monoclonal antibody or fragment thereof as described herein and a pharmaceutically acceptable carrier or excipient.

Aspects of the invention are also drawn to a nucleic acid encoding the isolated monoclonal antibody or fragment thereof as described herein.

In embodiments, the nucleic acid comprises a nucleic acid sequence according to SEQ ID NO: 9 and/or SEQ ID NO: 10, SEQ ID NO: 20 and/or SEQ ID NO: 21, or a sequence that is at least 80% identical thereto.

Aspects of the invention are also drawn towards a vector comprising the nucleic acid as described herein, and/or an isolated cell comprising the vector.

Still further, aspects of the invention are drawn towards an isolated monoclonal antibody or fragment thereof that binds to human intestinal alkaline phosphatase (hIAP) comprising a heavy chain variable region encoded by a nucleic acid sequence according to SEQ ID NO: 9 or a sequence that is at least 80% identical thereto, and/or a light chain variable region encoded by a nucleic acid sequence according to SEQ ID NO: 10 or a sequence that is at least 80% identical thereto.

Aspects of the invention are further drawn towards an isolated monoclonal antibody or fragment thereof that binds to human intestinal alkaline phosphatase (hIAP) comprising a heavy chain variable region encoded by a nucleic acid sequence according to SEQ ID NO: 20 or a sequence that is at least 80% identical thereto, and/or a light chain variable region encoded by a nucleic acid sequence according to SEQ ID NO: 21 or a sequence that is at least 80% identical thereto.

Also, aspects of the invention are drawn to an isolated cell comprising one or more polynucleotide(s) encoding the isolated monoclonal antibody or fragment thereof as described herein.

Aspects of the invention are drawn towards a method of detecting or diagnosing an iAP associated gastrointestinal disorder in a subject in need thereof comprising administering to a subject a therapeutically effective amount of the hIAP antibody or fragment thereof as described herein.

Aspects of the invention are also drawn towards a method of treating or preventing an iAP associated gastrointestinal disease in a subject in need thereof comprising administering to a subject a therapeutically effective amount of the hIAP antibody or fragment thereof as described herein.

In embodiments, the iAP associated disease comprises a gastrointestinal disease, non-limiting examples of which comprise the gastrointestinal disease comprises colitis, inflammatory bowel disease (IBD), gastritis, gastroenteritis, pyloric stenosis, gastric cancer, gastrointestinal cancer, colon cancer, infectious diarrhea, fecal impaction, constipation, intestinal obstruction and pseudoobstruction, or malabsorption.

Aspects of the invention are also drawn towards a kit comprising the isolated monoclonal antibody or fragment thereof as described herein.

Other objects and advantages of this invention are readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows the affinity and specificity of recombinant, monoclonal antibody CDI005E8 for human intestinal alkaline phosphatase. Panel A: Measurement of absorbance from horseradish peroxidase signal on secondary antibody was used to determine Kd of rmAb CDI005E8 to 2 μg/mL of peptide antigen 2-hsALPI coated onto microwell plate. Measurements were made in triplicate; average and standard deviation are shown in the log binding curve. Panel B, Top panel: Monoclonal antibody CDI005E8 identified human intestinal alkaline phosphatase in infant stool and did not cross react with purified, human placenta-like (PL) alkaline phosphatase (lane 1); purified, human tissue-nonspecific (TN) alkaline phosphatase (lane 3); or purified, human germ cell (GC) alkaline phosphatase. A total of 50 ng of each alkaline phosphate protein (placental, tissue non-specific, and germ cell) were loaded in lanes 1, 3, and 6 respectively. In lanes 4 and 5, 1 μg of infant stool sample or small intestinal lysate was loaded, respectively. Concentration of primary antibody was 20 nM CDI005E8 (top panel) or 5 nM polyclonal anti-intestinal alkaline phosphatase (Panel B, bottom panel; Heath et al. JAMA Netw Open 2 (11), e19114996); concentration of goat anti-rabbit secondary antibody was 1 nM for both blots.

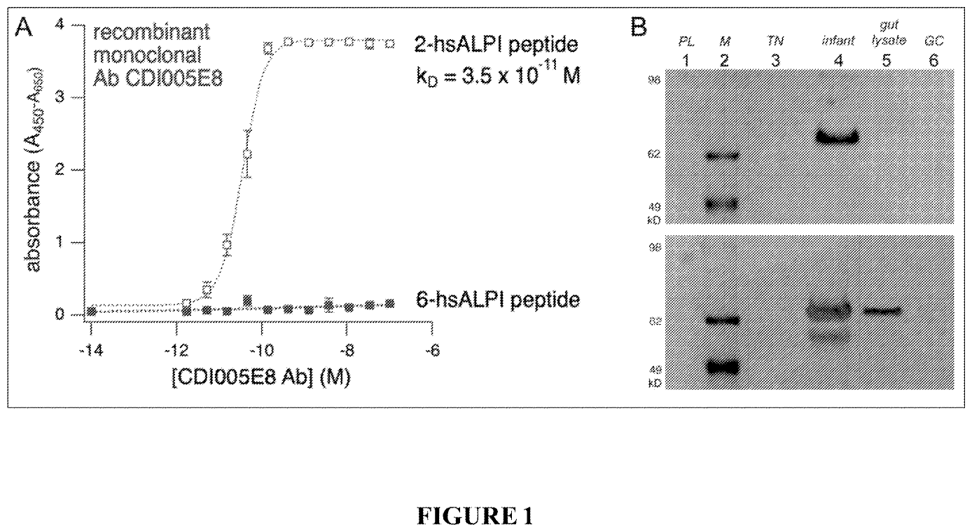

FIG. 2 shows the predicted three-dimensional model of the Fv region and of its antigen-binding pocket. (Panel A) Ribbon diagram of the heavy and light chains of CDI005E8. SAbPred was used to model the Fv structure of CDI005E8. The antibody sequence was numbered using ANARCI. Templates for the VH and VL framework regions were chosen from SAbDab and oriented with respect to each other using ABangle. The VH and VL orientations were found to be the same as in PDB ID 5v6m. SCRWL4 was used to predict the CDR conformations of side chains whose coordinates cannot be copied directly from a template structure. (Panel B) The CDR regions (H1-H3 & L1-L3) are shown in different blue shades. The Kabat guidelines were used for the CDR regions shown (also see Table 4). FREAD was used with CDR specific databases to predict the CDR conformations. (Panel C) Predicted paratope residues are shown in both chain width and heatmap color, in which the widest main chain diameter and red are associated with the highest scoring residue. Ala65 of the heavy chain (H2) in CDI005E8 sequence has the highest paratope score and is shown in red. Calculations were performed on Antibody i-Patch portal on the SAbPred website. Full description of the method can be found at Krawcyzk et al. Protein Engineering, Design & Selection 26, 621-629.

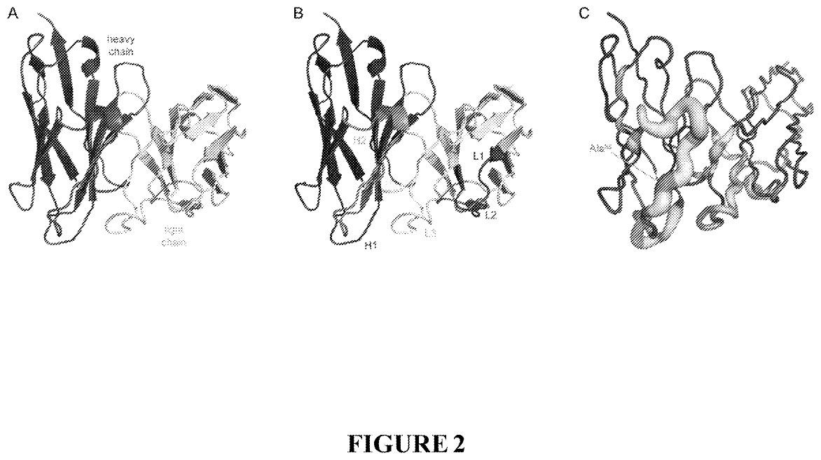

FIG. 3 is a schematic showing the summary of predicted epitope identification on antigen target.

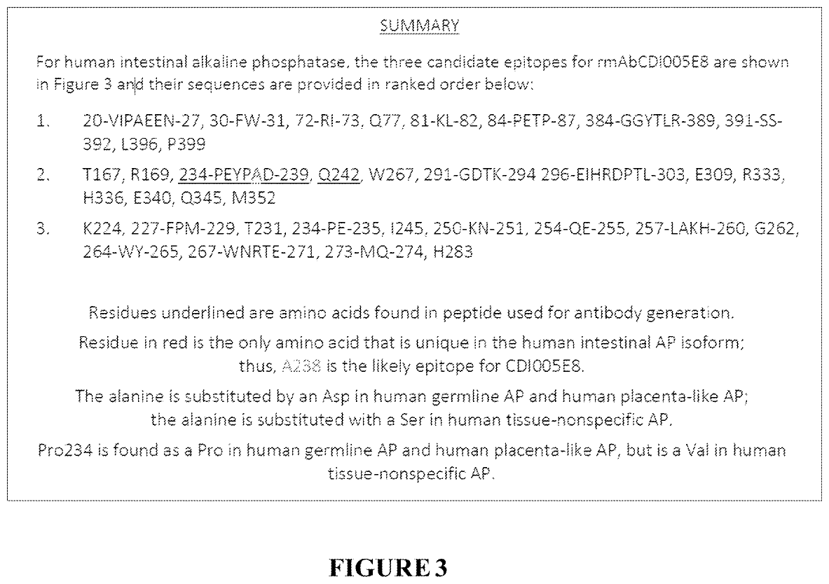

FIG. 4 shows the predicted human intestinal alkaline phosphatase epitope for rmAb CDI05E8. (panel A) Overlay of top three ranked, human iAP epitopes, shown in AlphaFold predicted iAP structure, for rmAb CDI005E8. (panel B) Epitope rank 1 in olive. (panel C) Epitope rank 2 in yellow. This epitope, which was predicted without prior knowledge of antigen, includes residues in peptide used to antibody generation. (panel D) Epitope rank 3 in orange. Calculations were performed on EpiPred portal on the SAbPrep server. Reference for the epitope identification is Krawczyk et al. Bioinformatics 30, 2288-2294.

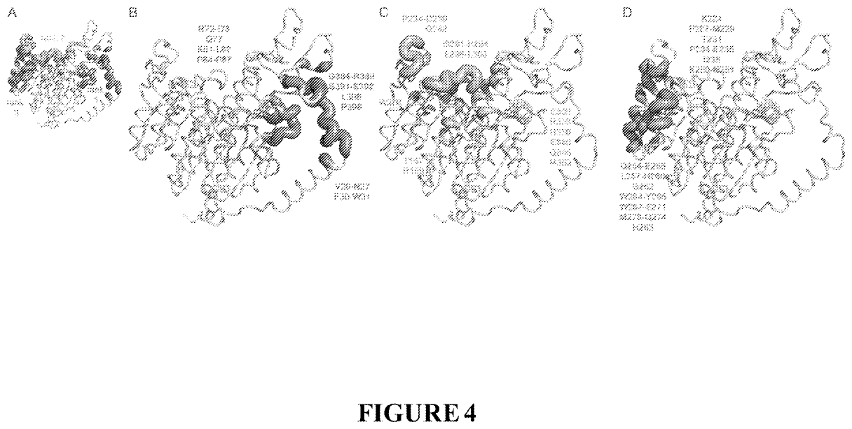

FIG. 5 shows the three-dimensional structures of alkaline phosphatase in (panel A) monomeric and (panel B) dimeric forms. (panel A) predicted human intestinal alkaline phosphatase structure from AlphaFold. Alphafold.ebi.ac.uk.entry/P09923 determination. Pymol file name-AF-P09923-F1-model-v2 human iAP.pse. (panel B) Initial evaluation of alkaline phosphatase from existing structure of human intestinal alkaline phosphatase homologs. Shown are human placenta-like alkaline phosphatase (PDB lew2) and rat intestinal alkaline phosphatase (PDB 4kjg).

FIG. 6 shows spatial location of candidate antigen peptide in human intestinal alkaline phosphatase. (panel A) Initial set and (panel B) second set of candidate peptides to serve as antigen for monoclonal antibodies. Peptide sequence is shown in spherical representation on a modeled dimeric structure of predicted human intestinal alkaline phosphatase from Alphafold (red cartoon, Alphafold.ebi.ac.uk. entry/P09923 determination) and human placenta-like alkaline phosphatase (grey ribbon, PDB lew2). Peptide has been omitted from the human placenta-like alkaline phosphatase for clarity.

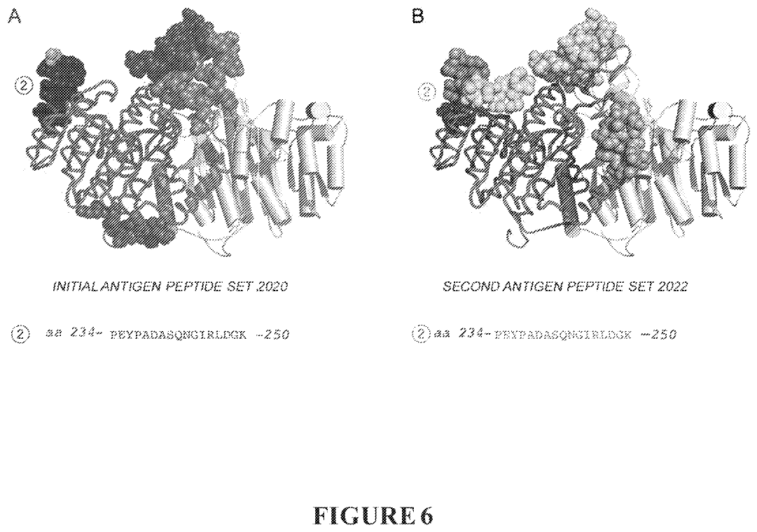

FIG. 7 shows a glycopeptide coverage map for rmAb CDI005E8. Amino Acids (AA) in bold and underlined are fragments that were chosen for RA calculations. AA in red are N-glycosylation sites.

FIG. 8 shows a representative MS2 spectrum of glycopeptide found in rmAb CDI005E8. Peaks representing HexNAc (3) Hex (3) structure on the following peptide EQQFNSTIR (pos. 304) are shown. Glycoform structures are based on total mass and known biosynthetic pathways, and do not reflect sequencing or compositional analysis.

FIG. 9 shows MALDI spectrum of N-glycans identified in rmAb CDI005E8. For the major N-glycans m/z is given together with cartoon representation of the structure.

FIG. 10 shows the affinity and specificity of recombinant, monoclonal antibody CDI022E3 for human intestinal alkaline phosphatase.

### DETAILED DESCRIPTION

### Abbreviations and Definitions

Detailed descriptions of one or more embodiments are provided herein. It is to be understood, however, that the invention may be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Intestinal Alkaline Phosphatase (iAP) Antibodies

Using a single 100-150 mg stool sample from three healthy human donors, 234 human proteins, secreted from the gastrointestinal tract, were identified. Of these, a core proteome of 57 proteins, common between these three human individuals, was identified.

Intestinal alkaline phosphatase (iAP) is expressed in small intestinal enterocytes, co-secreted into the intestinal lumen and systemic circulation and plays an integral role in maintaining gut barrier function by detoxifying bacterial lipopolysaccharides and maintaining microbial homeostasis. As the primary alkaline phosphatase in stool, iAP has been identified as one of the 57 proteins in the core human stool proteome.

Aspects of the invention provide isolated antibodies specific against iAP. The term “isolated” as used herein with respect to cells, nucleic acids, such as DNA or RNA, can refer to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and will not be found in the natural state. “Isolated” can also refer to cells or polypeptides which are isolated from other cellular proteins or tissues. Isolated polypeptides can include both purified and recombinant polypeptides. These antibodies represent a new class of monoclonal antibodies against iAP. For example, isolated cells can be mammalian cells, such as HEK293 cells or CHO cells.

Embodiments as described herein comprise recombinant monoclonal iAP antibodies. “Recombinant” as it pertains to polypeptides (such as antibodies) or polynucleotides can refer to a form of the polypeptide or polynucleotide that does not exist naturally, a non-limiting example of which can be created by combining polynucleotides or polypeptides that will not normally occur together.

The amino acid sequence of monoclonal iAP antibodies are provided herein, in addition to an exemplary wildtype IgG constant region useful in combination with the VH and VL sequences provided herein (see Table 1A-B); the amino acid sequences of the heavy and light chain complementary determining regions CDRs of the iAP antibodies are underlined (CDR1), underlined and bolded (CDR2), or underlined, italicized, and bolded (CDR3)

TABLE 1A
CDI005E8 Ab Variable Region amino acid sequences
V_{H} chain of CDI005E8
METGLRWLLLVAVLKGVQCQSLEESGGGLVQPGASLTLTCTASGESFTNNYWIYWVRQAPG
KGLEWIGSVIISSSSAYYASWAKGRFTITKPSSTTVTLQVTSLTAADTATYFCVTGGILWG
PGTLVTVSSGQPKAPSVFPLAPCCGDTPSSTVTLGCLVKGYLPEPVTVTWNSGTLINGVRT
FPSVRQSSGLYSLSSVVSVTSSSQPVTCNVAHPATNTKVDKTVAPSTCSKPMCPPPELPGG
PSVFIFPPKPKDTLMISRTPEVTCVVVDVSQDDPEVOFTWYINNEQVRTARPPLREQQENS
TIRVVSTLPIAHQDWLRGKEFKCKVHNKALPAPIEKTISKARGQPLEPKVYTMGPPREELS
SRSVSLTCMINGFYPSDISVEWEKNGKAEDNYKTTPTVLDSDGSYFLYSKLSVPTSEWQRG
DVFTCSVMHEALHNHYTQKSISRSPGK
(SEQ ID NO: 1)
V_{L} chain of CDI005E8
MDTRAPTQLLGLLLLWLPGARCDVVMTQTPASVEAAVGGTVTIKCQASQSIGTALAWYQQK
PGQPPKRLIYKASTLTSGVPSRFKGSGSGTDFTLTISDLECADAATYYCQCTVDDSSSGSS
YGNAFGGGTEVVVRGDPVAPTVLIFPPAADQVATGTVTIVCVANKYFPDVTVTWEVDGTTQ
TTGIENSKT PQNSADCTYNLSSTLTLTSTQYNSHKEYTCKVTQGTTSVVQSFNRGDC
(SEQ ID NO: 2)

TABLE 1B
CDI022E3 Ab Variable Region amino acid sequences
VH chain of CDI022E3
METGLRWLLLVAVLKGVQCQSLEESGGRLVTPGTPLTLTCTVSGIDLSTYAMGWVRQAPG
EGLEWIGTIGVSGSTYYASWAKGRFTISKTSTTVDLKMTSPATEDTATYFCARGSVWGPG
TLVTVSLGQPKAPSVFPLAPCCGDTPSSTVTLGCLVKGYLPEPVTVTWNSGTLINGVRTF
PSVRQSSGLYSLSSVVSVTSSSQPVTCNVAHPATNTKVDKTVAPSTCSKPMCPPPELPGG
PSVFIFPPKPKDTLMISRTPEVTCVVVDVSQDDPEVQFTWYINNEQVRTARPPLREQQFN
STIRVVSTLPIAHQDWLRGKEFKCKVHNKALPAPIEKTISKARGQPLEPKVYTMGPPREE
LSSRSVSLTCMINGFYPSDISVEWEKNGKAEDNYKTTPTVLDSDGSYFLYSKLSVPTSEW
QRGDVFTCSVMHEALHNHYTQKSISRSPGK
(SEQ ID NO: 20)
VL chain of CDI022E3
MDTRAPTQLLGLLLLWLPGATFAQVLTQTPASVEAAVGGTVTIKCQASQSISTWLAWYQQ
KPGQAPKRLIYSASTLASGVSSRFKGSGSGTEFTLTISDLECADAATYYCRCSYGKSYGE
AFGGGTEVVVKGDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQSENRGDC
(SEQ ID NO: 21)

The amino acid sequences of the heavy and light chain complementary determining regions of the iAP antibodies are shown in Table 2A-B:

TABLE 2
A. Heavy chain (V_{H}) and Light chain (V_{L}) complementary determining
regions (CDRs) of CDI005E8

| Sequence ID | V_{H} CDR1 | V_{H} CDR2 | V_{H} CDR3 |
|---|---|---|---|
| CDI005E8 | CTASGFSFTNNYWIYWV | LEWIGSVIISSSSA | CVTGGILWGPGT |
| Heavy Chain | (SEQ ID NO: 3) | YYASWAKGRFT | (SEQ ID NO: 5) |
| | | (SEQ ID NO: 4) | |
| Sequence ID | V_{L} CDR1 | V_{L} CDR2 | V_{L} CDR3 |
| CDI005E8 | COASQSIGTALAWYQ | IYKASTLTS | CTVDDSSSGSSYGNA |
| Light Chain | (SEQ ID NO: 6) | (SEQ ID NO: 7) | FGGG |
| | | | (SEQ ID NO: 8) |

B. Heavy chain (V_{H}) and Light chain (V_{L}) complementary determining
regions (CDRs) of CDI022E3

| Sequence ID | V_{H} CDR1 | V_{H} CDR2 | V_{H} CDR3 |
|---|---|---|---|
| CDI022E3 | TVSGIDLSTYAMG | TIGVSGSTY | ARGSV |
| Heavy Chain | (SEQ ID NO: 22) | (SEQ ID NO: 23) | (SEQ ID NO: 24) |
| | | TIGVSGSTYYASWAKG | |
| | | (SEQ ID NO: 31) | |
| Sequence ID | V_{L} CDR1 | V_{L} CDR2 | V_{L} CDR3 |
| CDI022E3 | QASQSISTWLA | YSASTLAS | RCSYGKSYGEA |
| Light Chain | (SEQ ID NO: 25) | (SEQ ID NO: 26) | (SEQ ID NO: 27) |

The nucleic acid sequence of the monoclonal iAP antibody is provided herein, in addition to an exemplary wildtype IgG constant region useful in combination with the VH and VL sequences provided herein (see Table 3A-B):

TABLE 3A
CDI005E8 Ab Variable Region nucleic acid sequences
V_{H} chain of CDI005E8
ATGGAGACTGGGCTGCGCTGGCTTCTCCTGGTCGCTGTGCTCAAAGGTGTCCAGTGTCAGT
CGTTGGAGGAGTCCGGGGGAGGCCTGGTCCAGCCTGGGGCATCCCTGACACTCACCTGCAC
AGCCTCTGGATTCTCCTTCACTAACAACTATTGGATATATTGGGTCCGCCAGGCTCCAGGG
AAGGGGCTGGAGTGGATCGGATCCGTGATTATTAGTAGTAGTAGCGCATACTACGCGAGCT
GGGCGAAAGGCCGATTCACCATCACCAAACCCTCGTCGACCACGGTGACTCTGCAGGTGAC
CAGTCTGACAGCCGCGGACACGGCCACCTATTTCTGTGTGACAGGTGGTATCTTATGGGGC
CCAGGCACCCTGGTCACCGTCTCCTCAGGGCAACCTAAGGCTCCATCAGTCTTCCCACTGG
CCCCCTGCTGCGGGGACACACCCAGCTCCACGGTGACCCTGGGCTGCCTGGTCAAAGGCTA
CCTCCCGGAGCCAGTGACCGTGACCTGGAACTCGGGCACCCTCACCAATGGGGTACGCACC
TTCCCGTCCGTCCGGCAGTCCTCAGGCCTCTACTCGCTGAGCAGCGTGGTGAGCGTGACCT
CAAGCAGCCAGCCCGTCACCTGCAACGTGGCCCACCCAGCCACCAACACCAAAGTGGACAA
GACCGTTGCGCCCTCGACATGCAGCAAGCCCATGTGCCCACCCCCTGAACTCCCGGGGGGA
CCGTCTGTCTTCATCTTCCCCCCAAAACCCAAGGACACCCTCATGATCTCACGCACCCCCG
AGGTCACATGCGTGGTGGTGGACGTGAGCCAGGATGACCCCGAGGTGCAGTTCACATGGTA
CATAAACAACGAGCAGGTGCGCACCGCCCGGCCGCCGCTACGGGAGCAGCAGTTCAACAGC
ACGATCCGCGTGGTCAGCACCCTCCCCATCGCGCACCAGGACTGGCTGAGGGGCAAGGAGT
TCAAGTGCAAAGTCCACAACAAGGCACTCCCGGCCCCCATCGAGAAAACCATCTCCAAAGC
CAGAGGGCAGCCCCTGGAGCCGAAGGTCTACACCATGGGCCCTCCCCGGGAGGAGCTGAGC
AGCAGGTCGGTCAGCCTGACCTGCATGATCAACGGCTTCTACCCTTCCGACATCTCGGTGG
AGTGGGAGAAGAACGGGAAGGCAGAGGACAACTACAAGACCACGCCGACCGTGCTGGACAG
CGACGGCTCCTACTTCCTCTACAGCAAGCTCTCAGTGCCCACGAGTGAGTGGCAGCGGGGC
GACGTCTTCACCTGCTCCGTGATGCACGAGGCCTTGCACAACCACTACACGCAGAAGTCCA
TCTCCCGCTCTCCGGGTAAATAG
(SEQ ID NO: 9)
V_{L} chain of CDI005E8
ATGGACACGAGGGCCCCCACTCAGCTGCTGGGGCTCCTGCTGCTCTGGCTCCCAGGTGCCA
GATGTGATGTTGTGATGACCCAGACTCCAGCCTCCGTGGAGGCAGCTGTGGGAGGCACAGT
CACCATCAAGTGCCAGGCCAGTCAGAGCATTGGCACTGCATTAGCCTGGTATCAGCAGAAA
CCAGGGCAGCCTCCCAAGCGCCTGATCTACAAGGCATCCACTCTGACATCTGGGGTCCCAT
CGCGGTTCAAAGGCAGTGGATCTGGGACAGATTTCACTCTCACCATCAGCGACCTGGAGTG
TGCCGATGCTGCCACTTACTACTGTCAATGTACTGTTGATGATAGTAGTAGTGGTAGTAGT
TATGGTAATGCTTTCGGCGGAGGGACCGAGGTGGTGGTCAGAGGTGATCCAGTTGCACCTA
CTGTCCTCATCTTCCCACCAGCTGCTGATCAGGTGGCAACTGGAACAGTCACCATCGTGTG
TGTGGCGAATAAATACTTTCCCGATGTCACCGTCACCTGGGAGGTGGATGGCACCACCCAA
ACAACTGGCATCGAGAACAGTAAAACACCGCAGAATTCTGCAGATTGTACCTACAACCTCA
GCAGCACTCTGACACTGACCAGCACACAGTACAACAGCCACAAAGAGTACACCTGCAAGGT
GACCCAGGGCACGACCTCAGTCGTCCAGAGCTTCAATAGGGGTGACTGTTAG
(SEQ ID NO: 10)

TABLE 3B
CDI022E3 Ab Variable Region nucleic acid sequences
V_{H} chain of CDI022E3
ATGGAGACTGGGCTGCGCTGGCTTCTCCTGGTCGCTGTGCTCAAAGGTGTCCAGTGTCAG
TCGCTGGAGGAGTCCGGGGGTCGCCTGGTCACGCCTGGGACACCCCTGACACTCACCTGC
ACAGTCTCTGGAATCGACCTCAGTACCTATGCAATGGGCTGGGTCCGCCAGGCTCCAGGG
GAGGGGCTGGAATGGATCGGGACCATTGGAGTTAGTGGTAGCACATACTACGCGAGCTGG
GCGAAAGGCCGATTCACCATCTCCAAAACCTCGACCACGGTGGATCTCAAAATGACCAGT
CCGGCAACCGAGGACACGGCCACCTATTTCTGTGCCAGAGGAAGCGTCTGGGGCCCAGGC
ACCCTGGTCACCGTCTCCTTGGGGCAACCTAAGGCTCCATCAGTCTTCCCACTGGCCCCC
TGCTGCGGGGACACACCCAGCTCCACGGTGACCCTGGGCTGCCTGGTCAAAGGCTACCTC
CCGGAGCCAGTGACCGTGACCTGGAACTCGGGCACCCTCACCAATGGGGTACGCACCTTC
CCGTCCGTCCGGCAGTCCTCAGGCCTCTACTCGCTGAGCAGCGTGGTGAGCGTGACCTCA
AGCAGCCAGCCCGTCACCTGCAACGTGGCCCACCCAGCCACCAACACCAAAGTGGACAAG
ACCGTTGCGCCCTCGACATGCAGCAAGCCCATGTGCCCACCCCCTGAACTCCCGGGGGGA
CCGTCTGTCTTCATCTTCCCCCCAAAACCCAAGGACACCCTCATGATCTCACGCACCCCC
GAGGTCACATGCGTGGTGGTGGACGTGAGCCAGGATGACCCCGAGGTGCAGTTCACATGG
TACATAAACAACGAGCAGGTGCGCACCGCCCGGCCGCCGCTACGGGAGCAGCAGTTCAAC
AGCACGATCCGCGTGGTCAGCACCCTCCCCATCGCGCACCAGGACTGGCTGAGGGGCAAG
GAGTTCAAGTGCAAAGTCCACAACAAGGCACTCCCGGCCCCCATCGAGAAAACCATCTCC
AAAGCCAGAGGGCAGCCCCTGGAGCCGAAGGTCTACACCATGGGCCCTCCCCGGGAGGAG
CTGAGCAGCAGGTCGGTCAGCCTGACCTGCATGATCAACGGCTTCTACCCTTCCGACATC
TCGGTGGAGTGGGAGAAGAACGGGAAGGCAGAGGACAACTACAAGACCACGCCGACCGTG
CTGGACAGCGACGGCTCCTACTTCCTCTACAGCAAGCTCTCAGTGCCCACGAGTGAGTGG
CAGCGGGGCGACGTCTTCACCTGCTCCGTGATGCACGAGGCCTTGCACAACCACTACACG
CAGAAGTCCATCTCCCGCTCTCCGGGTAAATAG
(SEQ ID NO: 28)
V_{L} chain of CDI022E3
ATGGACACGAGGGCCCCCACTCAGCTGCTGGGGCTCCTGCTGCTCTGGCTCCCAGGTGCC
ACATTTGCCCAAGTGCTGACCCAGACTCCAGCCTCCGTGGAGGCAGCTGTGGGAGGCACA
GTCACCATCAAGTGCCAGGCCAGTCAGAGCATTAGCACCTGGTTAGCCTGGTATCAGCAG
AAACCAGGGCAGGCTCCCAAGCGCCTGATCTATTCTGCATCCACTCTGGCATCTGGGGTC
TCATCGCGGTTCAAAGGCAGTGGATCTGGGACAGAGTTCACTCTCACCATCAGCGACCTG
GAGTGTGCCGATGCTGCCACTTACTACTGTCGATGTAGTTATGGTAAAAGTTATGGTGAG
GCTTTCGGCGGAGGGACCGAGGTGGTGGTCAAAGGTGATCCAGTTGCACCTACTGTCCTC
ATCTTCCCACCAGCTGCTGATCAGGTGGCAACTGGAACAGTCACCATCGTGTGTGTGGCG
AATAAATACTTTCCCGATGTCACCGTCACCTGGGAGGTGGATGGCACCACCCAAACAACT
GGCATCGAGAACAGTAAAACACCGCAGAATTCTGCAGATTGTACCTACAACCTCAGCAGC
ACTCTGACACTGACCAGCACACAGTACAACAGCCACAAAGAGTACACCTGCAAGGTGACC
CAGGGCACGACCTCAGTCGTCCAGAGCTTCAATAGGGGTGACTGTTAG
(SEQ ID NO: 29)

As described herein, an iAP protein or a derivative, fragment, analog, homolog or ortholog thereof, can be utilized as an immunogen in the generation of antibodies that immunospecifically binds iAP. As described herein, the immunogens used in the generation of antibodies to iAP comprise PEYPADASQNGIRLDGK (SEQ ID NO: 11) and/or PGYVENSGVRPDVNESESGSPDY (SEQ ID NO: 30). These sequences don't necessarily need to be displayed as accessible epitopes on native iAP protein, and antibodies that recognize these linear epitopes do not necessarily need to be generated at appreciable levels when using native iAP protein as the immunogen. Second, even if these epitopes are displayed on the surface of native iAP in an accessible fashion, these sequences can be blocked in clinical samples by interactions with specific host antibodies or interactions with other host proteins. Without wishing to be bound by theory, these confounding effects are eliminated when diagnostic samples are digested to liberate target peptides. Thus, the use of peptide-specific antibodies to recognize target peptides produced by specific digestion of diagnostic samples, has synergistic effects to increase the detection sensitivity and specificity for a target biomarker.

Those skilled in the art will recognize that it is possible to determine, without undue experimentation, if a monoclonal antibody has the same specificity as a monoclonal antibody of the invention by ascertaining whether the former prevents the latter from binding to iAP protein or fragment thereof. Without wishing to be bound by theory, if the monoclonal antibody being tested competes with the monoclonal antibody of the invention, as shown by a decrease in binding by the monoclonal antibody of the invention, then the two monoclonal antibodies can bind to the same, or to a closely related, epitope. Competition assays are well known to those of skill in the art.

Another way to determine whether a monoclonal antibody has the specificity of a monoclonal antibody of the invention is to pre-incubate the monoclonal antibody of the invention with an iAP polypeptide with which it is normally reactive, and then add the monoclonal antibody being tested to determine if the monoclonal antibody being tested is inhibited in its ability to bind iAP. If the monoclonal antibody being tested is inhibited then, it has the same, or functionally equivalent, epitopic specificity as the monoclonal antibody of the invention.

As used herein, the term “epitope” can include any protein determinant that can specifically bind to an immunoglobulin, a single-chain variable fragment (scFv), or a T cell receptor. Epitopic determinants can consist of chemically active surface groupings of molecules such as amino acids or sugar side chains and can have specific three-dimensional structural characteristics, as well as specific charge characteristics. For example, antibodies can be raised against N-terminal or C-terminal peptides of a polypeptide.

Without wishing to be bound by theory, the antibodies and fragments described herein bind a distinct epitope. As described herein, a peptide is used as the immunogen in the production of the antibodies, and therefore the epitope can comprise a linear amino acid sequence therein. In embodiments, the linear amino acid sequence is 2, 3, 4, 5, 6, 7, 8, 9, 10, 11, 12, 13, 14, 15, 16, 17, 18, 19, 20, or greater than 20 amino acids in length. In some embodiments, the linear amino acid sequence can be 11, 12, 13, 14, 15, 16, 17, 18, 19, 20, 21, 22, 23, 24, or 25 amino acids in length. In embodiments, the antibodies described herein can bind to the same or overlapping segment of the peptide immunogen. In other embodiments, the antibodies described herein can bind distinct, non-overlapping peptide sequences.

If two or more antibodies bind to the same or overlapping segment of a peptide immunogen, they can be considered to compete with each other for binding to the peptide immunogen. The term “competes with,” when referring to a pair of antibodies, can refer to a first antibody that detectably competes with a second antibody (or other molecule) in a binding assay using, for example, recombinant immunogen or cell-surface expressed immunogens. For example, a first antibody can block or attenuate the binding of a second antibody, if this second bound to an epitope that overlapped with the epitope that was bound by the first antibody. Further, if the epitope of the second antibody is directly adjacent to the epitope bound by the first antibody, then the first antibody can block or attenuate the binding of the second antibody by spatial exclusion or steric effects.

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kon) and the “off rate constant” (Koff) can be determined by calculation of the concentrations and the actual rates of association and dissociation. (See Nature 361:186-87 (1993)). The ratio of Koff/Kon allows for the cancellation of parameters not related to affinity, and is equal to the dissociation constant Kd. (See, for example, Davies et al. (1990) Annual Rev Biochem 59:439-473). An antibody of the invention is said to specifically bind to iAP epitope when the equilibrium binding constant (Kd) is ≤1 μM, ≤100 nM, ≤10 nM, or ≤100 μM to about 1 μM, as measured by assays such as radioligand binding assays or similar assays known to those skilled in the art. For example, the Kd of CDI005E8 Ab is 7.11±1.01 pM, when using 2 μg/mL (1.1 μM) of peptide antigen 2-hsALPI. This was surprising considering the hydrophobic iAP epitope and the hydrophobic antibody paratope are an unusual combination to elicit picomolar binding affinities.

Monoclonal antibodies as described herein can undergo affinity maturation, for example, to increase the strength or affinity of immunological binding. “Affinity maturation” can refer to a process in which an antibody is evolved from a reference antibody (also referred to as a parent antibody), such as by mutation of one or more amino acid residues, to have increased activity for a target antigen than a corresponding form of the reference antibody has for the same target antigen. Hence, the evolved antibody is optimized compared to the reference or template antibody.

Reference to an affinity matured antibody can refer to an antibody that has an increased activity for a target antigen relative to a reference antibody. For example, the affinity matured antibody can exhibit increased binding to the target antigen compared to the reference or parent antibody. The affinity matured antibody can bind to the same epitope as the reference antibody.

An optimized antibody can refer to an antibody, or portion thereof, that has an increased activity for a target protein or antigen compared to a reference antibody, for example, improved binding affinity for a target protein and/or an improved functional activity. The antibody can be optimized by virtue of one or more amino acid modifications (amino acid deletion, replacement or insertion) compared to a parent antibody not containing the one or more amino acid modifications. An activity, for example binding affinity, can be increased by at or about 1.5-fold to 1000-fold, such as at least or about 2-fold to 100-fold, for example at or about 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold or more compared to an activity of the parent antibody (e.g. germline antibody not containing the modification(s)).

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen that is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25 28).

The antibodies described herein can be detected by appropriate assays, e.g., conventional types of immunoassays. For example, an assay can be performed in which iAP or fragment thereof is affixed to a solid phase. Incubation is maintained for a sufficient period of time to allow the antibody in a sample to bind to the immobilized polypeptide on the solid phase. After this first incubation, the solid phase is separated from the sample. The solid phase is washed to remove unbound materials and interfering substances such as non-specific proteins which can also be in the sample. The solid phase containing the antibody of interest bound to the immobilized polypeptide is subsequently incubated with a second, labeled antibody or antibody bound to a coupling agent such as biotin or avidin. This second antibody can be another anti-iAP antibody or another antibody. Labels for antibodies are well-known in the art and include radionuclides, enzymes (e.g. maleate dehydrogenase, horseradish peroxidase, glucose oxidase, catalase), fluors (fluorescein isothiocyanate, rhodamine, phycocyanin, fluorescarmine), biotin, and the like. The labeled antibodies are incubated with the solid and the label bound to the solid phase is measured. These and other immunoassays can be easily performed by those of ordinary skill in the art.

The term “monoclonal antibody” or “MAb” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in the molecules of the population. MAbs contain an antigen binding site that can be immunoreactive with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, can be immunized with an immunizing agent to elicit lymphocytes that can produce antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are required for uses described herein, or spleen cells or lymph node cells can be used if non-human mammalian sources required for uses described herein. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59 103). Immortalized cell lines can be transformed mammalian cells, such as myeloma cells of rodent, bovine and human origin. For example, rat or mouse myeloma cell lines can be employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas can include hypoxanthine, aminopterin, and thymidine (“HAT medium”), which substances prevent the growth of HGPRT deficient cells.

Immortalized cell lines can be those that fuse efficiently, support stable high-level expression of antibody by the selected antibody producing cells, and are sensitive to a medium such as HAT medium. Immortalized cell lines can be murine myeloma lines, which can be obtained, for instance, from the Salk Institute Cell Distribution Center, San Diego, California and the American Type Culture Collection, Manassas, Virginia. Human myeloma and mouse human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51 63)).

The culture medium in which the hybridoma cells are cultured can then be assayed for the presence of monoclonal antibodies directed against the antigen. The binding specificity of monoclonal antibodies produced by the hybridoma cells is determined by immunoprecipitation or by an in vitro binding assay, such as radioimmunoassay (RIA) or enzyme linked immunoabsorbent assay (ELISA). Such techniques and assays are known in the art.

“Affinity” or “binding affinity” can refer to the strength with which an antibody molecule or portion thereof binds to an epitope on a target protein or antigen. Affinity can be measured by the equilibrium binding constant (K_{A}) or the equilibrium dissociation constant (K_{D}). The low affinity antibody-antigen interactions are weak and the molecules tend to separate rapidly, while the high affinity antibody-antigen binding is strong and the molecules remain in the bound state for an extended period of time. For example, the affinity of the antibody for the target protein is, in equilibrium binding constant (K_{A}), about 10 6 M⁻¹ or more, about 10⁷ M⁻¹ or more, about 10⁸ M⁻¹ or more, or about 10⁹ M⁻¹ 10¹⁰ M⁻¹, 10¹¹ M⁻¹ or 10¹² M⁻¹. Furthermore, the antibody can have an equilibrium dissociation constant (K_{D}) of about 10⁻⁴ M to 10⁻⁵ M, about 10⁻⁵ M to 10⁻⁶ M, about 10⁻⁶ M to 10⁻⁷ M, about 10⁻⁷ M to 10⁻⁸ M, about 10⁻⁸ M, about 10⁻⁹ M, about 10⁻¹⁰ M, about 10⁻¹¹ M, about 10⁻¹² M or about 10⁻¹³ M or about greater than 10⁻¹³ M. Low dissociation constants can also be characterized. Without wishing to be bound by theory, a low dissociation constant can be that the antibody is characterized by a higher binding affinity. For example, antibodies with a dissociation constant of nM, or nM or less, are considered as high affinity antibodies. Such affinities are known in the art and can be determined, e.g., by equilibrium dialysis; by surface plasmon resonance (SPR) using a BIAcore instrument according to the general procedures outlined by the manufacturer; radioimmunoassays using radiolabeled target antigens; or by other methods known to the person skilled in the art. Analysis can be performed using the method of ScL, 51:660 (1949). Further, the binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI 1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that can specifically bind to genes encoding the heavy and light chains of murine antibodies). The hybridoma cells of the invention can be a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812 13 (1994)) or by covalently joining to the immunoglobulin coding sequence all or part of the coding sequence for a non immunoglobulin polypeptide. Such a non immunoglobulin polypeptide can be substituted for the constant domains of an antibody described herein or can be substituted for the variable domains of one antigen combining site of an antibody of the invention to create a chimeric bivalent antibody.

Fully human antibodies are antibody molecules in which the entire sequence of both the light chain and the heavy chain, including the CDRs, arise from human genes. Such antibodies are termed “human antibodies”, or “fully human antibodies” herein. Human monoclonal antibodies can be prepared by using trioma technique; the human B cell hybridoma technique (see Kozbor, et al., 1983 Immunol Today 4:72); and the EBV hybridoma technique to produce human monoclonal antibodies (see Cole, et al., 1985 In: MONOCLONAL ANTIBODIES AND CANCER THERAPY, Alan R. Liss, Inc., pp. 77 96).

Human monoclonal antibodies can be utilized and can be produced by using human hybridomas (see Cote, et al., 1983. Proc Natl Acad Sci USA 80:2026 2030) or by transforming human B cells with Epstein Barr Virus in vitro (see Cole, et al., 1985 In: MONOCLONAL ANTIBODIES AND CANCER THERAPY, Alan R. Liss, Inc., pp. 77 96).

“Humanized antibodies” can be antibodies from a non-human species (such as mouse), whose amino acid sequences (for example, in the CDR regions) have been modified to increase their similarity to antibody variants produced naturally in humans. Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant can be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes the antibody described herein, or produces the antibody of the invention.

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779 783 (1992); Lonberg et al., Nature 368 856 859 (1994); Morrison, Nature 368, 812 13 (1994); Fishwild et al, Nature Biotechnology 14, 845 51 (1996); Neuberger, Nature Biotechnology 14, 826 (1996); and Lonberg and Huszar, Intern. Rev. Immunol. 13 65 93 (1995).

Human antibodies can additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides the modifications is then obtained as progeny by crossbreeding intermediate transgenic animals containing fewer than the full complement of the modifications. An embodiment of such a nonhuman animal can be a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, are disclosed in PCT publication WO 99/53049.

The antibody can be expressed by a vector containing a DNA segment encoding the single chain antibody described herein. A vector can refer to a system that can carry a foreign gene and can be rendered in a host cell. These include plasmid vectors, recombinant viral vectors, recombinant bacterial vectors, pseudovirions, virus-like particles, and the like.

These can include vectors, liposomes, naked DNA, adjuvant-assisted DNA, gene gun, catheters, etc. Vectors include chemical conjugates such as described in WO 93/64701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

The “recombinant viral vector” can refer to a vector that can carry a foreign gene based on a recombinant virus, and includes, for example, a recombinant adenovirus vector, a recombinant poxvirus vector, a baculovirus vector, and the like.

The “recombinant bacterial vector” can refer to a vector that can carry a foreign gene constructed based on a recombinant bacterium, and includes, for example, a Listeria vector, an attenuated Salmonella vector, and the like.

Vectors can include viral vectors, fusion proteins and chemical conjugates. Retroviral vectors include moloney murine leukemia viruses. DNA viral vectors can be used. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cell's cytoplasm. Avipoxvirus vectors result in only a short-term expression of the nucleic acid. Adenovirus vectors, adeno-associated virus vectors and herpes simplex virus (HSV) vectors can be used to introduce the nucleic acid into neural cells. The adenovirus vector results in a shorter-term expression (about 2 months) than adeno-associated virus (about 4 months), which in turn is shorter than HSV vectors. The vector chosen will depend upon the target cell. The introduction can be by standard techniques, e.g. infection, transfection, transduction or transformation. Examples of modes of gene transfer include e.g., naked DNA, CaPO4 precipitation, DEAE dextran, electroporation, protoplast fusion, lipofection, cell microinjection, and viral vectors.

The vector can be employed to target essentially any target cell. For example, stereotaxic injection can be used to direct the vectors (e.g. adenovirus, HSV) to a required location for uses described herein. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al., Proc. Natl. Acad. Sci. USA 91:2076-2080 (1994); Morrison et al., Am. J. Physiol. 266:292-305 (1994)).

These vectors can be used to express large quantities of antibodies that can be used in a variety of ways. For example, to detect the presence of iAP in a sample. The antibody can also be used to try to bind to and disrupt the function of iAP.

In an embodiment, the antibodies of the invention are full-length antibodies, containing an Fc region similar to wild-type Fc regions that bind to Fc receptors.

Heteroconjugate antibodies are also within the scope of the invention. Heteroconjugate antibodies are composed of two covalently joined antibodies. The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl 4 mercaptobutyrimidate and those disclosed, for example, in U.S. Pat. No. 4,676,980.

In embodiments, the antibodies of the invention have a specified percentage identity or similarity to the amino acid or nucleotide sequences of the anti-iAP antibodies described herein. For example, “homology” or “identity” or “similarity” refers to sequence similarity between two peptides or between two nucleic acid molecules. Homology can be determined by comparing a position in each sequence, which can be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-iAP antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-iAP antibodies described herein. Sequence identity or similarity to the nucleic acids and proteins of the invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the invention.

“Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a nucleic acid sequence. It can be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of the anti-iAP antibodies disclosed herein can exhibit increased cross-reactivity to iAP in comparison to an unmodified iAP antibody.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the antigen and does not react with other polypeptides.

The terms “antibody fragment” or “antigen-binding fragment”, as used herein, is a portion of an antibody such as F(ab′) 2, F (ab) 2, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising a VL or VH domain, fragments produced by a Fab expression library, and anti-idiotypic (anti-Id) antibodies.

A “single-chain variable fragment” or “scFv” refers to a fusion protein of the variable regions of the heavy (VH) and light chains (VL) of immunoglobulins. A single chain Fv (“scFv”) polypeptide molecule is a covalently linked VH: VL heterodimer, which can be expressed from a gene fusion including VH- and VL-encoding genes linked by a peptide-encoding linker. (See Huston et al. (1988) Proc Nat Acad Sci USA 85 (16): 5879-5883). In some aspects, the regions are connected with a short linker peptide of ten to about 25 amino acids. The linker can be rich in glycine for flexibility, as well as serine or threonine for solubility, and can connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Antibody molecules obtained from humans fall into five classes of immunoglobulins: IgG, IgM, IgA, IgE and IgD, which differ from one another by the nature of the heavy chain in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains can be joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as kappa or lambda (K, 2). Each heavy chain class can be bound with a kappa or lambda light chain. In general, the light and heavy chains are covalently bonded to each other, and the “tail” portions of the two heavy chains are bonded to each other by covalent disulfide linkages or non-covalent linkages when the immunoglobulins are generated by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” VH and VL regions, which contain the CDRs, as well as frameworks (FRs) of the iAP antibodies are shown in Table 4.

The six CDRs in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, the FR regions, show less inter-molecular variability. The framework regions largely adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of definition to refer to a CDR of an antibody or variants thereof is intended to be within the scope of the term as defined and used herein. The appropriate amino acid residues which encompass the CDRs as defined by each of the references cited herein are set forth in the table herein as a comparison. The exact residue numbers which encompass a CDR will vary depending on the sequence and size of the CDR. Those skilled in the art can routinely determine which residues comprise a CDR given the variable region amino acid sequence of the antibody.

| CDR | Kabat Numbering | Chothia Numbering |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table described herein, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10⁻¹⁷ residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three dimensional antigen-binding site. This quaternary antibody structure forms the antigen-binding site at the end of each arm of the Y. Epitopic determinants can consist of chemically active surface groupings of molecules such as amino acids or sugar side chains and have specific three dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3). In one embodiment, the antibodies can be directed to human intestinal alkaline phosphatase (iAP) having NCBI Reference No: NP_001622.2 (528 amino acid residues in length) comprising the amino acid sequence of SEQ ID NO: 12:

| | |
|---|---|
| 1 | MQGPWVLLLL GLRLQLSLGV IPAEEENPAF |
| | WNRQAAEALD AAKKLQPIQK VAKNLILELG |
| 61 | DGLGVPTVTA TRILKGQKNG KLGPETPLAM |
| | DREPYLALSK TYNVDRQVPD SAATATAYLC |
| 121 | GVKANFQTIG LSAAARFNQC NTTRGNEVIS |
| | VMNRAKQAGK SVGVVTTTRV QHASPAGTYA |
| 181 | HTVNRNWYSD ADMPASARQE GCQDIATQLI |
| | SNMDIDVILG GGRKYMFPMG TPDPEYPADA |
| 241 | SQNGIRLDGK NLVQEWLAKH QGAWYVWNRT |
| | ELMQASLDQS VTHLMGLFEP GDTKYEIHRD |
| 301 | PTLDPSLMEM TEAALRLLSR NPRGFYLEVE |
| | GGRIDHGHHE GVAYQALTEA VMEDDAIERA |
| 361 | GQLTSEEDTL TLVTADHSHV FSFGGYTLRG |
| | SSIFGLAPSK AQDSKAYTSI LYGNGPGYVE |
| 421 | NSGVRPDVNE SESGSPDYQQ QAAVPLSSET |
| | HGGEDVAVFA RGPQAHLVHG VQEQSFVAHV |
| 481 | MAFAACLEPY TACDLAPPAC TTDAAHPVAA |
| | SLPLLAGTLL LLGASAAP |

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ allows for the cancellation of all parameters not related to affinity, and is equal to the equilibrium binding constant, K_{D}. (See, for example, Davies et al. (1990) Annual Rev Biochem 59:439-473). An antibody of the invention can specifically bind to a iAP epitope when the equilibrium binding constant (K_{D}) is ≤1 μM, ≤10 μM, ≤10 nM, ≤10 μM, or ≤100 μM to about 1 μM, as measured by kinetic assays such as radioligand binding assays or similar assays known to those skilled in the art, such as BIAcore or Octet (BLI). For example, in some embodiments, the K_{D} is between about 1E⁻¹² M and a K_{D} about 1E⁻¹¹ M. In some embodiments, the K_{D} is between about 1E⁻¹¹ M and a K_{D} about 1E⁻¹⁰ M. In some embodiments, the K_{D} is between about 1E⁻¹⁰ M and a K_{D} about 1E⁻⁹ M. In some embodiments, the K_{D} is between about 1E⁻⁹ M and a K_{D} about 1E⁻⁸ M. In some embodiments, the K_{D} is between about 1E⁻⁸ M and a K_{D} about 1E⁻⁷ M. In some embodiments, the K_{D} is between about 1E⁻⁷ M and a K_{D} about 1E⁻⁶ M. For example, in some embodiments, the K_{D} is about 1E⁻¹² M while in other embodiments the K_{D} is about 1E⁻¹¹ M. In some embodiments, the K_{D} is about 1E⁻¹⁰ M while in other embodiments the K_{D} is about 1E⁻⁹ M. In some embodiments, the K_{D} is about 1E⁻⁸ M while in other embodiments the K_{D} is about 1E⁻⁷ M. In some embodiments, the K_{D} is about 1E⁻⁶ M while in other embodiments the K_{D} is about 1E⁻⁵ M. In some embodiments, for example, the K_{D} is about 3 E⁻¹¹ M, while in other embodiments the K_{D} is about 3E⁻¹² M. In some embodiments, the K_{D} is about 6E⁻¹¹ M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it will bind to a random, unrelated epitope.

For example, the iAP antibody can be monovalent or bivalent, and can comprise a single or double chain. Functionally, the binding affinity of the iAP antibody is within the range of 10 5M to 10⁻¹²M. For example, the binding affinity of the iAP antibody is from 10⁻⁶ M to 10⁻¹² M, from 10⁻⁷M to 10⁻¹² M, from 10⁻⁸ M to 10⁻¹² M, from 10⁻⁹M to 10⁻¹² M, from 10⁻⁵ M to 10⁻¹¹M, from 10⁻⁶ M to 10⁻¹¹ M, from 10⁻⁷M to 10⁻¹¹ M, from 10 8 M to 10⁻¹¹ M, from 10⁻⁹M to 10⁻¹¹ M, from 10⁻¹⁰ M to 10⁻¹¹ M, from 10⁻⁵ M to 10⁻¹⁰M, from 10⁻⁶ M to 10⁻¹⁰ M, from 10⁻⁷M to 10⁻¹⁰ M, from 10⁻⁸ M to 10⁻¹⁰M, from 10⁻⁹M to 10⁻¹⁰ M, from 10⁻⁵ M to 10⁻⁹M, from 10⁻⁶ M to 10⁻⁹M, from 10⁻⁷M to 10⁻⁹M, from 10⁻⁸ M to 10⁻⁹M, from 10⁻⁵ M to 10⁻⁸ M, from 10⁻⁶ M to 10⁻⁸ M, from 10⁻⁷M to 10⁻⁸ M, from 10⁻⁵ M to 10⁻⁷M, from 10⁻⁶ M to 10⁻⁷M, or from 10⁻⁵ M to 10⁻⁶ M.

An iAP protein, or a derivative, fragment, analog, homolog or ortholog thereof, can be utilized as an immunogen in the generation of antibodies that immunospecifically bind these protein components, e.g., amino acid residues comprising SEQ ID NO: 12 or a fragment thereof. An iAP protein or a derivative, fragment, analog, homolog, or ortholog thereof, coupled to a proteoliposome can be utilized as an immunogen in the generation of antibodies that immunospecifically bind these protein components.

Those skilled in the art will recognize that it is possible to determine, without undue experimentation, if a human monoclonal antibody has the same specificity as a human monoclonal antibody of the invention by ascertaining whether the former prevents the latter from binding to iAP. For example, if the human monoclonal antibody being tested competes with the human monoclonal antibody of the invention, as shown by a decrease in binding by the human monoclonal antibody of the invention, then the two monoclonal antibodies can bind to the same, or to a closely related, epitope.

Another way to determine whether a human monoclonal antibody has the specificity of a human monoclonal antibody of the invention is to pre-incubate the human monoclonal antibody of the invention with the iAP protein, with which it is normally reactive, and then add the human monoclonal antibody being tested to determine if the human monoclonal antibody being tested is inhibited in its ability to bind iAP. If the human monoclonal antibody being tested is inhibited then, in all likelihood, it has the same, or functionally equivalent, epitopic specificity as the monoclonal antibody of the invention. Screening of human monoclonal antibodies of the invention can be also carried out by utilizing iAP and determining whether the test monoclonal antibody is able to neutralize iAP.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen, which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in all the molecules of the population. MAbs contain an antigen binding site that can immunoreact with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, can be immunized with an immunizing agent to elicit lymphocytes that produce or can produce antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes for cells of human origin, or spleen cells or lymph node cells can be for non-human mammalian sources. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, such as myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas can include hypoxanthine, aminopterin, and thymidine (“HAT medium”), which substances prevent the growth of HGPRT-deficient cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, California) and the American Type Culture Collection (Manassas, Virginia). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that can bind to genes encoding the heavy and light chains of murine antibodies). The hybridoma cells of the invention serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies, for example, are antibody molecules in which the entire sequence of both the light chain and the heavy chain, including the CDRs, arise from human genes. Such antibodies are termed “human antibodies” or “fully human antibodies”. Human monoclonal antibodies, such as fully human and humanized antibodies, can be prepared by using trioma technique; the human B-cell hybridoma technique (see Kozbor, et al, 1983 Immunol Today 4:72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

“Humanized antibodies” can be antibodies from a non-human species (such as mouse), whose amino acid sequences (for example, in the CDR regions) have been modified to increase their similarity to antibody variants produced in humans. Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant can be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can eadily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes the antibody of the invention, or produces the antibody of the invention.

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28 (4/5): 489-498 (1991); Studnicka et al., Protein Engineering 7 (6): 805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (such as rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20).

In addition, antibodies (such as human antibodies) can also be produced using other techniques, including phage display libraries. (See Hoogenboom and Winter, J. Mol. Biol, 227:381 (1991); Marks et al., J. Mol. Biol, 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13:65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the modifications is then obtained as progeny by crossbreeding intermediate transgenic animals containing fewer than the full complement of the modifications. A non-limiting example of such a nonhuman animal is a mouse, and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publication nos. WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described herein. Vectors include, but are not limited to, chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used, and include moloney murine leukemia viruses. DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87:1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G . . . et al, Nat. Genet. 8:148 (1994).

Pox viral vectors introduce the gene into the cell's cytoplasm. Avipox virus vectors result in only a short term expression of the nucleic acid. Adenovirus vectors, adeno-associated virus vectors, and herpes simplex virus (HSV) vectors can be used for introducing the nucleic acid into neural cells. The adenovirus vector results in a shorter term expression (about 2 months) than adeno-associated virus (about 4 months), which in turn is shorter than HSV vectors. The vector chosen will depend upon the target cell and the condition being treated. The introduction can be by standard techniques, e.g. infection, transfection, transduction or transformation. Examples of modes of gene transfer include e.g., naked DNA, CaP04 precipitation, DEAE dextran, electroporation, protoplast fusion, lipofection, cell microinjection, and viral vectors.

The vector can be employed to target any target cell. For example, stereotaxic injection can be used to direct the vectors (e.g. adenovirus, HSV) to a location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of iAP in a sample.

In an embodiment, the antibodies described herein can be full-length antibodies, containing an Fc region similar to wild-type Fc regions that bind to Fc receptors.

Techniques can be adapted for the production of single-chain antibodies specific to an antigenic protein of the invention (See e.g., U.S. Pat. No. 4,946,778). In addition, methods can be adapted for the construction of Fab expression libraries (See e.g., Huse, et al, 1989 Science 246:1275-1281) to allow rapid and effective identification of monoclonal Fab fragments with specificity for a protein or derivatives, fragments, analogs or homologs thereof. Antibody fragments that contain the idiotypes to a protein antigen can be produced by techniques known in the art including, but not limited to: (i) an F(ab′) 2 fragment produced by pepsin digestion of an antibody molecule; (ii) an Fab fragment generated by reducing the disulfide bridges of an F(ab′) 2 fragment; (iii) an Fab fragment generated by the treatment of the antibody molecule with papain and a reducing agent and (iv) Fv fragments.

Heteroconjugate antibodies are also within the scope of the invention. Heteroconjugate antibodies are composed of two covalently joined antibodies. Such antibodies can, for example, target immune system cells to unwanted cells (see U.S. Pat. No. 4,676,980), and for treatment of HIV infection (See PCT Publication Nos. WO91/00360; WO92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in detecting or treating gastrointestinal disease. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176:1 191-1 195 (1992) and Shopes, J. Immunol., 148:2918-2922 (1992)). Alternatively, an antibody can be engineered that has dual Fc regions and can thereby have enhanced complement lysis and ADCC capabilities. (See Stevenson et al, Anti-Cancer Drug Design, 3:219-230 (1989)). In one embodiment, the antibody of the invention has modifications of the Fc region, such that the Fc region does not bind to the Fc receptors. For example, the Fc receptor is Fcγ receptor. Antibodies with modification of the Fc region such that the Fc region does not bind to Fcγ, but still binds to neonatal Fc receptor are useful as described herein.

In certain embodiments, an antibody of the invention can comprise an Fc variant comprising an amino acid substitution which alters the antigen-independent effector functions of the antibody, such as the circulating half-life of the antibody. Such antibodies exhibit increased or decreased binding to FcRn when compared to antibodies lacking these substitutions, therefore, have an increased or decreased half-life in serum, respectively. Fc variants with improved affinity for FcRn can have longer serum half-lives, and such molecules have use applications in methods of treating mammals where long half-life of the administered antibody is beneficial, e.g., to treat a chronic disease or disorder. In contrast, Fc variants with decreased FcRn binding affinity can have shorter halt-lives, and such molecules are also useful, for example, for administration to a mammal where a shortened circulation time can be advantageous, e.g., for in vivo diagnostic imaging or in situations where the starting antibody has toxic side effects when in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can include those applications for localizing the antibody to the brain, kidney, and/or liver. In embodiments, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation is a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In some embodiments, the antibody has a N-linked glycosylation site in the heavy chain at amino acid position 304 (EU numbering). In some embodiments, the antibody has a N-linked glycosylation site in the light chain at amino acid position 202 (EU numbering). In another embodiment, the antibody has an amino acid substitution near or within a glycosylation motif, for example, an N-linked glycosylation motif that contains the amino acid sequence NXT or NXS. In an embodiment, the antibody comprises an Fc variant with an amino acid substitution at amino acid position 228 or 299 (EU numbering). In other embodiments, the antibody comprises an IgG1 or IgG4 constant region comprising an S228P and a T299A mutation (EU numbering). In some embodiments, the N-linked glycosylation site of the antibody described herein is removed. For example, a deglycosylated mutant of the heavy chain of the antibody described herein can comprise a N304A mutation (EU numbering). For example, a deglycosylated mutant of the light chain of the antibody described herein can comprise a N202A mutation (EU numbering).

Exemplary amino acid substitutions which confer reduced or altered glycosylation are described in PCT Publication No, WO05/018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG₄ antibody which is devoid of Fc-effector function thereby eliminating the potential for Fc mediated toxicity to the normal vital tissues and cells that express iAP. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238:1098 (1987). Carbon-14-labeled l-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved by direct condensation of existing side chains or by the incorporation of external bridging molecules. Many bivalent or polyvalent linking agents are useful in coupling protein molecules, such as the antibodies of the invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the coupling agents. (See Killen and Lindstrom, Jour. Immun. 133:1335-2549 (1984); Jansen et al., Immunological Reviews 62:185-216 (1982); and Vitetta et al, Science 238:1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, for example, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al, Proc. Natl. Acad. Sci. USA, 82:3688 (1985); Hwang et al, Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556. Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes can extruded through filters of defined pore size to yield liposomes with a given diameter. Fab′ fragments of the antibody of the invention can be conjugated to the liposomes as described in Martin et al, J. Biol. Chem., 257:286-288 (1982) via a disulfide-interchange reaction.

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. This invention provides for multispecific antibodies, such as bi-specific and trispecific antibodies, that recognize iAP and a second antigen and/or a third antigen. In one embodiment, multispecific antibodies (e.g., bi-specific antibodies and trispecific antibodies) can comprise iAP specific fusion proteins encompassing the antibodies described herein. In one embodiment, the bispecific and trispecific antibodies comprise iAP fusion proteins. For example, the fusion protein comprises an antibody comprising a variable domain or scFv unit and a ligand or antigen and/or a third ligand or antigen as described herein such that the resulting antibody recognizes an antigen and binds to the ligand-specific receptor. In one embodiment, the fusion protein further comprises a constant region, and/or a linker as described herein. Different formats of multispecific antibodies (e.g., bispecific antibodies and trispecific antibodies such as a fusion protein comprises an antibody that recognizes iAP and a ligand) are described herein. Different formats of bispecific or trispecific antibodies are also provided herein. In some embodiments, each of the anti-iAP fragment and the second antigen-specific fragment and/or the third antigen-specific fragment is each independently selected from a Fab fragment, a single-chain variable fragment (scFv), or a single-domain antibody. A bispecific or trispecific antibody of the invention can comprise a heavy chain and a light chain combination or scFv of the iAP antibodies described herein.

Multispecific antibodies (e.g., bispecific antibodies and trispecific antibodies) of the invention (for example, an anti-iAP-scFv fusion protein) can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker depicted herein (GGGGSGGGGS; “(G4S) 2”) can be generated with a longer G4S linker to improve flexibility. For example, the linker can also be “(G4S) 3” (e.g., GGGGGGGGSGGGGS); “(G4S) 4” (e.g., GGGGSGGGGSGGGGSGGGGS) (SEQ ID NO: 13); “(G4S) 5” (e.g., GGGGSGGGGSGGGGSGGGGSGGGGS) (SEQ ID NO: 14); “(G4S) 6” (e.g., GGGGSGGGGSGGGGSGGGGSGGGGSGGGGS) (SEQ ID NO: 15); “(G4S) 7” (e.g., GGGGSGGGGSGGGGSGGGGSGGGGSGGGGSGGGGS) (SEQ ID NO: 16); and the like. For example, use of the (G4S) 5 linker can provide more flexibility to a ligand described herein and can improve expression. In some embodiments, the linker can also be (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. Non-limiting examples of linkers known to those skilled in the art that can be used to construct the fusions described herein can be found in U.S. Pat. No. 9,708,412; U.S. Patent Application Publication Nos. US20180134789 and US20200148771; and PCT Publication No. WO2019051122 (each of which are incorporated by reference in their entireties).

In another embodiment, the multispecific antibodies (e.g., bispecific antibodies and trispecific antibodies such as anti-iAP-scFv fusions) can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens/ligands or three different antigens/ligands are mixed to promote heteroligation pairing, which is mediated through the engineered “knob into holes” of the CH3 domains.

In another embodiment the multispecific antibodies (e.g., bispecific antibodies and trispecific antibodies such as anti-iAP-scFv fusions) can be constructed through exchange of heavy-light chain dimers from two or more different antibodies to generate a hybrid antibody where the first heavy-light chain dimer recognizes iAP and the second heavy-light chain dimer recognizes a second antigen and/or third antigen. The mechanism for heavy-light chain dimer is similar to the formation of human IgG₄, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG₄ molecules. Heavy chain pairing is also stabilized further by interheavy chain disulfide bridges in the hinge region of the antibody. Specifically, in IgG₄, the hinge region contains the amino acid sequence Cys-Pro-Ser-Cys (in comparison to the stable IgG1 hinge region which contains the sequence Cys-Pro-Pro-Cys) at amino acids 226-230. This sequence difference of Serine at position 229 has been linked to the tendency of IgG₄ to form intrachain disulfides in the hinge region (Van der Neut Kolfschoten, M. et al, 2007, Science 317:1554-1557 and Labrijn, A. F. et al, 2011, Journal of Immunol 187:3238-3246).

The multispecific antibodies (e.g., bispecific antibodies and trispecific antibodies such as anti-iAP-scFv fusions) of the invention can be created through introduction of the R409 residue in the CH3 domain and the Cys-Pro-Ser-Cys sequence in the hinge region of antibodies that recognize iAP or a second and/or third antigen, so that the heavy-light chain dimers exchange to produce an antibody molecule with one heavy-light chain dimer recognizing iAP and the second heavy-light chain dimer recognizing a second and/or third antigen, wherein the second and/or third antigen (or ligand) is any antigen (or ligand) disclosed herein. Known IgG₄ molecules can also be altered such that the heavy and light chains recognize iAP or a second and/or third antigen, as disclosed herein. Use of this method for constructing the multispecific antibodies (e.g., bispecific antibodies and trispecific antibodies such as anti-AP-scFv fusions) of the invention can be beneficial due to the intrinsic characteristic of IgG₄ molecules wherein the Fc region differs from other IgG subtypes in that it interacts poorly with effector systems of the immune response, such as complement and Fc receptors expressed by certain white blood cells. This specific property makes these IgG₄-based multispecific antibodies (e.g., bispecific antibodies and trispecific antibodies such as anti-iAP-scFv fusions) attractive for therapeutic applications, in which the antibody is required to bind the target(s) and functionally alter the signaling pathways associated with the target(s), however not trigger effector activities.

The multispecific antibodies (e.g., bispecific antibodies and trispecific antibodies such as anti-iAP-scFv fusions) described herein can be engineered with a non-depleting heavy chain isotype, such as IgG₁-LALA or stabilized IgG₄ or one of the other non-depleting variants. In some embodiments, mutations are introduced to the constant regions of the bsAb such that the antibody dependent cell-mediated cytotoxicity (ADCC) activity of the bsAb is altered. For example, the mutation is a LALA mutation in the CH2 domain. In one aspect, the multispecific antibody (e.g., bispecific antibodies and trispecific antibodies such as anti-iAP-scFv fusions) contains mutations on one scFv unit of the heterodimeric multispecific antibody, which reduces the ADCC activity. In another aspect, the multispecific antibody (e.g., bispecific antibodies and trispecific antibodies such as anti-iAP-scFv fusions) contains mutations on both chains of the heterodimeric multispecific antibody, which completely ablates the ADCC activity. For example, the mutations introduced in one or both scFv units of the multispecific antibody (e.g., bispecific antibodies and trispecific antibodies such as anti-iAP-scFv fusions) are LALA mutations in the CH2 domain. These multispecific antibodies (e.g., bispecific antibodies and trispecific antibodies such as anti-iAP-scFv fusions) with variable ADCC activity can be optimized such that the multi-specific antibodies exhibit maximal selective killing towards cells that express one antigen that is recognized by the multispecific antibody, however, exhibits minimal killing towards the second antigen that is recognized by the multispecific antibody.

The multispecific antibodies (e.g., bispecific antibodies and trispecific antibodies such as anti-iAP-scFv fusions) disclosed herein can be useful in treatment of chronic infections, diseases, or medical conditions, for example, gastrointestinal disease.

### Pharmaceutical Compositions

Antibodies of the invention specifically binding an iAP protein or fragment thereof can be administered for the treatment of a gastrointestinal disease in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

A specific dosage and treatment regimen for a patient will depend upon a variety of factors, including the antibody administered, variant or derivative thereof used, the patient's age, body weight, general health, sex, and diet, and the time of administration, rate of excretion, drug combination, and the severity of the disease being treated. Judgment of such factors by medical caregivers is within the ordinary skill in the art. The amount will also depend on the individual patient to be treated, the route of administration, the type of formulation, the characteristics of the compound used, the severity of the disease, and the effect intended. The amount used can be determined by pharmacological and pharmacokinetic principles well known in the art.

A therapeutically effective amount of an antibody of the invention can be the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. The dosage administered to a subject (e.g., a patient) of the antigen-binding polypeptides described herein can be 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration can be used. Further, the dosage and frequency of administration of antibodies of the disclosure can be reduced by enhancing uptake and tissue penetration (e.g., into the brain) of the antibodies by modifications such as, for example, lipidation. Ranges for therapeutically effective dosing of an antibody or antibody fragment of the invention can be, by way of nonlimiting examples, from about 0.1 mg/kg body weight to about 50 mg/kg body weight. Dosing frequencies can range, for example, from twice daily to once a week.

Where antibody fragments are used, the smallest inhibitory fragment that specifically binds to the binding domain of the target protein can be used. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90:7889-7893 (1993)). The formulation can also contain more than one active compound as necessary for the indication being treated, for example, those with complementary activities that do not adversely affect each other. Alternatively, or in addition, the composition can comprise an agent that enhances its function, such as, for example, a cytotoxic agent, cytokine (e.g. IL-15), chemotherapeutic agent, or growth-inhibitory agent. Such molecules are suitably in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid allow for the release of molecules for over 100 days, certain hydrogels release proteins for shorter time periods.

The antibodies or agents of the invention (also referred to herein as “active compounds”), and derivatives, fragments, analogs and homologs thereof, can be incorporated into pharmaceutical compositions suitable for administration. Such pharmaceutical compositions can comprise the antibody or agent and a pharmaceutically acceptable carrier.

As used herein, the term “pharmaceutically acceptable carrier” can include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is included herein. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated herein, as required, followed by filtered sterilization. For example, dispersions are prepared by incorporating the active compound into a sterile vehicle that contains a basic dispersion medium and the required other ingredients from those enumerated herein. In the case of sterile powders for the preparation of sterile injectable solutions, methods of preparation are vacuum drying and freeze-drying that yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein refers to physically discrete units suited as unitary dosages for the subject to be treated; each unit containing a predetermined quantity of active compound calculated to produce a therapeutic effect in association with the required pharmaceutical carrier. The specification for the dosage unit forms of the invention are dictated by and directly dependent on the unique characteristics of the active compound and the therapeutic effect to be achieved, and the limitations inherent in the art of compounding such an active compound for the treatment of individuals.

The pharmaceutical compositions can be included in a container, pack, or dispenser together with instructions for administration.

### Method of Detecting an iAP Associated Gastrointestinal Disease

Antibodies of the invention specifically binding an iAP protein, or a fragment thereof, can be administered for the detection of an iAP associated gastrointestinal disease or disorder. An “iAP-associated disease or disorder” includes disease states and/or symptoms associated with a disease state, where increased levels of iAP and/or activation of cellular signaling pathways involving iAP are found. Exemplary iAP-associated diseases or disorders include, but are not limited to diseases involving the gastrointestinal tract or region thereof, namely the esophagus, stomach, small intestine, large intestine or rectum, as well as organs and tissues associated with digestion, e.g., the pancreas, the gallbladder, and the liver. In some embodiments, the gastrointestinal diseases can be colitis, inflammatory bowel disease (IBD), gastritis, gastroenteritis, pyloric stenosis, gastric cancer, infectious diarrhea, fecal impaction, constipation, intestinal obstruction and pseudoobstruction, or malabsorption. In addition to necrotizing enterocolitis (NEC), non-limiting examples of types of colitis comprise adult necrotizing enterocolitis (ANEC), pseudomembranous enterocolitis, infectious colitis, very early onset inflammatory bowel disease, ulcerative colitis, Crohn's disease, ischemic colitis, and radiation colitis. In some embodiments, the gastrointestinal disease can be caused by a microorganism, such as a Gram positive bacterium, a Gram negative bacterium, a protozoa, a fungus, a DNA virus, a RNA virus, or a reverse transcribing virus.

Non-limiting examples of disease-causing bacteria include: Bacillus anthracis, Bacillus cereus, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitides, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholera, Yersinia pestis, Yersinia enterocolitica, Yersinia pseudotuberculosis.

Non-limiting examples of disease-causing protozoa include Plasmodium falciparum (malaria), Toxoplasma gondii (toxoplasmosis), Leishmania species (leishmaniases), Trypanosoma brucei (African sleeping sickness), Trypanosoma cruzi (Chagas disease), and Giardia intestinalis (giardiasis).

Non-limiting examples of disease-causing fungi include Candida albicans, Aspergillus fumigatus, Aspergillus flavus, Cryptococcus neoformans, Cryptococcus gattii, Histoplasma capsulatum, Pneumocystis carinii, Stachybotrys chartarum.

Non-limiting examples of disease-causing viruses include Adenovirus, Coxsackievirus, Epstein-Barr virus, Hepatitis A virus, Hepatitis B virus, Hepatitis C virus, Herpes simplex virus, type 1, Herpes simplex virus, type 2, Cytomegalovirus, Human herpesvirus, type 8, HIV, Influenza virus, Measles virus, Mumps virus, Human papillomavirus, Parainfluenza virus, Poliovirus, Rabies virus, Respiratory syncytial virus, Rubella virus, Varicella-zoster virus, coronavirus (such as human coronavirus NL63 (HCoV-NL63), severe acute respiratory syndrome coronavirus (SARS-COV), Middle East respiratory syndrome coronavirus (MERS-COV), or severe acute respiratory syndrome coronavirus-2 (SARS-COV-2)).

In embodiments, the antibody can contain a detectable label. Antibodies can be polyclonal or monoclonal. An intact antibody, or a fragment thereof (e.g., Fab, scFv, or F (ab) 2) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids within a subject. Included within the usage of the term “biological sample”, therefore, is feces, such as stool, a tissue (e.g., blood), cell (e.g., hematopoietic cell such as hematopoietic stem cell, leukocyte, or reticulocyte, stem cell, or plasma cell), vesicle, biomolecular aggregate or platelet from the subject. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Antibodies directed against an iAP protein (or a fragment thereof) can be used in methods known within the art relating to the localization and/or quantitation of an iAP protein (e.g., for use in measuring levels of the iAP protein within appropriate physiological samples, for use in diagnostic methods, for use in imaging the protein, and the like). In a given embodiment, antibodies specific to an iAP protein, or derivative, fragment, analog or homolog thereof, that contain the antibody derived antigen binding domain, are utilized as pharmacologically active compounds (referred to herein as “therapeutics”).

An antibody of the invention specific for an iAP protein can be used to isolate an iAP polypeptide by standard techniques, such as immunoaffinity, chromatography or immunoprecipitation. Antibodies directed against an iAP protein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P or ³H.

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate). Immunoconjugates can also comprise an antibody conjugated with a label. In some embodiments, the label comprises a chromagen. Each chromogen listed is reactive with the corresponding enzyme to produce a signal which reports the presence of the immunoconjugate. The superscript notation (*) indicates that the chromogen fluoresces, rather than produces a color change. Non-limiting examples of chromagens and their corresponding enzyme are listed herein.

| Chromagen | Enzyme |
|---|---|
| *4-Methylumbelliferyl phosphate | alkaline phosphatase |
| * bis (4-Methylumbelliferyl phosphate) | or |
| 3-0-methylfluorescein | acid phosphatase |
| *Flavone-3-diphosphate tetraammonium salt | |
| p-nitrophenyl phosphate 2Na | |
| *Tyramine hydrochloride | peroxidase |
| *3-(p-hydroxyphenyl) Propionic acid | |
| *p-Hydroxyphenethyl alcohol | |
| 2,2′-Azino-Di-3-Ethylbenzothiazoline | |
| Sulfonic acid (ABTS) | |
| ortho-phenylenediamine 2HCl | |
| O-dianisidine | |
| *5-aminosalicylic acid | |
| p-cresol | |
| 3,3′-dimethyloxybenzidine | |
| 3-methyl-2-benzothiazolinehydrazone | |
| 3,3′,5,5′-Tetramethylbenzidine (TMB) | |
| o-nitrophenyl-β-D-galactopyranoside | β-D-galactosidase |
| 4-methylumbelliferyl phosphate | |
| β-D-galactoside | |
| 2,2′-Azino-Di-3-Ethylbenzothiazoline | Glucose oxidase |
| Sulfonic acid (ABTS) | |

In some embodiments, the label comprises a binding moiety (e.g., biotin-avidin, biotin-streptavidin or sugar-lectin, myc tag, his tag and the like). In some embodiments, the label comprises a colored dye compound, such as a fluorescent dye. Non-limiting examples of fluorescent dye compounds include fluorescein, ethidium bromide, rhodamine, Texas Red, Phycoerythrin (RPE), and cyanine. In some embodiments, the label comprises a fluorescent compound (e.g., GFP, RFP, YFP, BFP, and the like). In some embodiments, the label consists of a nanoparticle, where the size, composition and geometry of the particle can determine the absorption and emission properties of the nanoparticle, which can be altered by interaction with other particles with overlapping emission and absorption spectra that can undergo other interactions (e.g. plasmonic resonance). In some embodiments, the label comprises a chemiluminescent compound (e.g., N-(4-Aminobutyl)-N-ethylisoluminol, 4-Aminophthalhydrazide, Disodium 2-chloro-5-(4-methoxyspiro[1,2-dioxetane-3,2′-(5-chlorotricyclo[3.3.1.13.7]decan])-4-yl]-1-phenyl phosphate). In some embodiments, the label comprises an enzymatic compound (e.g., β-D-galactosidase or peroxidase).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

For example, the antibody can be coupled (i.e., physically linked) to a detectable substance for the detection of iAP or fragment thereof. Non-limiting examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. Covalent binding can be used, and achieved by, for example, direct condensation of existing side chains or by the incorporation of external bridging molecules. Many bivalent or polyvalent linking agents are useful in coupling protein molecules, such as the antibodies of the invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of coupling agents. (See Killen and Lindstrom, Jour. Immun. 133:1335-2549 (1984); Jansen et al., Immunological Reviews 62:185-216 (1982); and Vitetta et al., Science 238:1098 (1987)).

Linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Linkers can include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is prone to disruption in vitro, resulting in less conjugate available. Sulfo-NHS, for example, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82:3688 (1985); Hwang et al., Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556.

Useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the required diameter for uses described herein. Fab′ fragments of the antibody of the invention can be conjugated to the liposomes as described in Martin et al., J. Biol. Chem., 257:286-288 (1982) via a disulfide-interchange reaction.

Aspects of the invention comprise measuring or detecting biomarkers of gastrointestinal diseases in biological samples. Gastrointestinal diseases can refer to diseases involving the gastrointestinal tract, namely the esophagus, stomach, small intestine, large intestine and rectum, and the accessory organs of digestion, the liver, gallbladder, and pancreas. For example, such diseases can result from infectious, autoimmune, and physiological states. Non-limiting examples of gastrointestinal diseases include colitis, inflammatory bowel disease (IBD), gastritis, gastroenteritis, pyloric stenosis, gastric cancer, infectious diarrhea, fecal impaction, constipation, intestinal obstruction and pseudo-obstruction, or malabsorption. In addition to necrotizing enterocolitis (NEC), non-limiting examples of types of colitis comprise adult necrotizing enterocolitis (ANEC), pseudomembranous enterocolitis, infectious colitis, very early onset inflammatory bowel disease, ulcerative colitis, Crohn's disease, ischemic colitis, and radiation colitis.

In embodiments, the gastrointestinal disease is necrotizing enterocolitis (NEC). NEC is an acquired gastrointestinal disease often seen in pre-term infants. In NEC, bacteria invade the wall of the intestine, causing local infection and inflammation. NEC is characterized by high mortality and long-term morbidity, including short gut syndrome, recurrent infection, nutritional deficiency and neurodevelopmental delay. Despite an overall net decrease in mortality for premature infants, there has been an increase in NEC-associated deaths. NEC is often difficult to diagnose and manage, due to initial nonspecific symptomatology and rapid deterioration.

In addition to necrotizing enterocolitis seen in neonates and preterm neonates, necrotizing enterocolitis can also affect non-neonates. For example, necrotizing enterocolitis in non-neonates, such as adults, can result from inflammatory mediators; nutritional disorders, such as anorexia or significant weight loss; gastrointestinal dysfunction; alcoholism; malabsorption; agents that block intestinal proteases; smoking; circulatory disturbances, such as reduced mesenteric blood flow, bowel ischemia, atherosclerosis of the bowel arteries; cholelithiasis; administration of drugs; immunological deficiencies, such as of the IgA secretory component or intestinal T-lymphocytes coupled with poor antibody response: fecal impaction or constipation: or infectious agents, such as bacterial infections, food borne infections and food borne illnesses.

Non-limiting examples of such drugs include those with anticholinergic properties, such as neuroleptics or phenothiazine-based neuroleptics, narcotics, inflammatory mediators, antidepressants, iron pills, laxatives, or antacids.

Non-limiting examples of such infectious agents comprise bacteria like Klebsiella, E. coli, Enterobacter, Pseudomonas, Clostridia and Staphylococcus epidermidis, viruses like Corona virus, Rota virus and Entero virus and rarely, fungi like Candida albicans. Enteropathogenic viruses can infect epithelial cells resulting in cell destruction, necrosis and intestinal perforation.

Constipation or fecal impact can have many different causes known to art, non-limiting examples of which include antacid medicines containing calcium or aluminum, changes in diet or activities, colon cancer, dairy products, eating disorders, neurological conditions, inactivity, dehydration, consuming fiber, overuse of laxatives, pregnancy, digestive disorders, resisting the urge to have a bowel movement, medications, stress, or hypothyroidism.

Embodiments can comprise measuring or detecting biomarkers of gastrointestinal diseases in biological samples. Biomarkers of the invention can be measured in different types of biological samples. Non-limiting examples of biological samples that can be used in methods of the invention, although not intended to be limiting, include stool, plasma, cord blood, neonatal blood, cerebral spinal fluid, tears, vomit, saliva, urine, feces, and meconium. In embodiments, a sample can be prepared to enhance detectability of the biomarkers. For example, a sample from the subject can be fractionated. Any method that enriches for a biomarker polypeptide of interest can be used. Sample preparations, such as prefractionation protocols, are optional and can or cannot be necessary to enhance detectability of biomarkers depending on the methods of detection used. For example, sample preparation can be unnecessary if an antibody that specifically binds a biomarker is used to detect the presence of the biomarker in a sample. Sample preparation can involve fractionation of a sample and collection of fractions determined to contain the biomarkers. Methods of prefractionation include, for example, size exclusion chromatography, ion exchange chromatography, heparin chromatography, affinity chromatography, sequential extraction, gel electrophoresis, mass spectrometry, and liquid chromatography.

The methods described herein can involve obtaining a biological sample from the subject, such as an infant. As used herein, the phrase “obtaining a biological sample” refers to any process for directly or indirectly acquiring a biological sample from a subject. For example, a biological sample can be obtained (e.g., at a point-of-care facility, such as a physician's office, a hospital, laboratory facility) by procuring a tissue or fluid sample (e.g., blood draw, marrow sample, spinal tap) from a subject. Alternatively, a biological sample can be obtained by receiving the biological sample (e.g., at a laboratory facility) from one or more persons who procured the sample directly from the subject. The biological sample can be, for example, feces, such as stool, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

The term “sample” can refer to any sample that can contain an analyte of interest, such as iAP or a fragment thereof. A sample can be a non-biological sample or a biological sample, and encompasses clinical specimens (diagnostic samples collected as part of standard clinical procedures). Non-biological samples include those prepared in vitro comprising varying concentrations of a target molecule of interest in solution. Biological samples include, without limitation, whole blood, lymph, serum, plasma, urine, saliva, sputum, breath extract (meaning exhaled air captured in a solution), bone marrow, aspirates (nasal, lung, bronchial, tracheal), eye fluid, amniotic fluid, feces other bodily fluids and secretions, cells, and tissue specimens and dilutions of them. As used herein, the term “body fluid” means any fluid that can be isolated from the body of an individual. For example, “body fluid” can include blood, plasma, serum, bile, saliva, urine, tears, perspiration, and the like. In embodiments, the sample comprises a body fluid sample from human; for example plasma or serum. For example, the bodily fluid can be obtained from a vertebrate mammal, such as, a human having, suspected of having, and/or at risk for developing, an infection. Any suitable biological sample can be used. For example, a biological sample can be a specimen obtained from a subject or can be derived from such a subject. A subject can provide a plurality of biological samples, including a solid biological sample, from for example, a biopsy or a tissue. In some cases, a sample can be a tissue section or cells that are placed in or adapted to tissue culture. A biological sample also can be a biological fluid such as urine, blood, plasma, serum, saliva, tears, or mucus, or such a sample absorbed onto a paper or polymer substrate. A biological sample can be further fractionated, for example, to a fraction containing a cell type(s). In some embodiments, a sample can be a combination of samples from a subject (e.g., a combination of a tissue and fluid sample). In some cases, serum or plasma is obtained from the subject using techniques known in the art.

The term “biological sample” can refer to tissues, cells and biological fluids isolated from a subject, as well as tissues, cells and fluids within a subject. Included within the usage of the term “biological sample”, therefore, is blood and a fraction or component of blood including blood serum, blood plasma, or lymph. The definition can also include a sample that has been manipulated in any way after its procurement, such as by treatment with reagents, solubilization, or enrichment for certain components, such as proteins or polynucleotides.

In embodiments, the biological sample can be isolated from or obtained from a subject. As used herein, the phrase “obtaining a biological sample” or “isolating a biological sample” can refer to any process for directly or indirectly acquiring a biological sample from a subject. For example, a biological sample can be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample can be obtained by receiving the biological sample (e.g., at a laboratory facility) from one or more persons who procured the sample directly from the subject. Further, the biological sample can be obtained, such as directly or indirectly, from an animal, such as for livestock or wildlife management efforts or veterinary care.

In one embodiment, the sample contains protein molecules from the subject. In one embodiment, the biological sample is a peripheral blood leukocyte sample isolated by conventional means from a subject. In another embodiment, the biological sample is blood serum isolated by conventional means from a subject.

As used herein, the terms “purification”, “purifying”, and “enriching” do not refer to removing materials from the sample other than the analyte(s) of interest. Instead, these terms refer to a procedure that enriches the amount of one or more analytes of interest relative to other components in the sample that can interfere with detection of the analyte of interest.

Purification of the sample by various means can allow relative reduction of one or more interfering substances, e.g., one or more substances that can or cannot interfere with the detection of selected analyte by mass spectrometry. Relative reduction as this term is used does not require that any substance, with the analyte of interest in the material to be purified, is entirely removed by purification.

The term “immunopurification” or “immunopurify” can refer to a purification procedure that utilizes antibodies, including polyclonal or monoclonal antibodies, such as those described herein, to enrich the one or more analytes of interest. Immunopurification can be performed using any of the immunopurification methods well known in the art. Often the immunopurification procedure utilizes antibodies bound, conjugated or otherwise attached to a solid support, for example a bead, column, well, tube, gel, capsule, particle or the like.

Immunopurification can include without limitation procedures often referred to in the art as immunoprecipitation, as well as procedures often referred to in the art as affinity chromatography or immunoaffinity chromatography.

The term “immunoparticle” can refer to a capsule, bead, gel particle or the like that has antibodies bound, conjugated or otherwise attached to its surface (for example, on and/or in the particle). In certain embodiments, immunoparticles are sepharose or agarose beads. In alternative embodiments, immunoparticles comprise glass, plastic or silica beads, or silica gel.

As used herein, the term “anti-iAP antibody” can refer to any polyclonal or monoclonal antibody that has an affinity for iAP, epitope thereof, or peptide fragment thereof. In various embodiments the specificity of the antibodies to chemical species other than iAP can vary; for example, in certain embodiments the anti-iAP antibodies are specific for iAP and thus have little or no affinity for chemical species other than iAP, whereas in other embodiments the anti-iAP antibodies are non-specific and thus bind certain chemical species other than iAP.

The antibodies and fragments as described herein can be utilized with any solid phase enrichment approach. The term “solid phase enrichment” or “solid phase extraction” or “SPE” can refer to a process in which a chemical mixture is separated into components as a result of the affinity of components dissolved or suspended in a solution (i.e., mobile phase) for a solid through or around which the solution is passed (i.e., solid phase). In some instances, as the mobile phase passes through or around the solid phase, components of the mobile phase can be retained by the solid phase resulting in a purification of the analyte in the mobile phase. In other instances, the analyte, such as iAP, can be retained by the solid phase, allowing components of the mobile phase to pass through or around the solid phase. In these instances, a second mobile phase is then used to elute the retained analyte off of the solid phase for further processing or analysis. SPE, including TFLC, can operate via a unitary or mixed mode mechanism. Mixed mode mechanisms utilize ion exchange and hydrophobic retention in the same column; for example, the solid phase of a mixed-mode SPE column can exhibit strong anion exchange and hydrophobic retention; or can exhibit strong cation exchange and hydrophobic retention.

To facilitate separation of the antibody-protein complex from unbound protein in the sample, the antibody can be linked to an agent that facilitates separation, such as a binding partner (e.g., biotin, oligonucleotide, aptamer), a solid support (such as a bead or matrix, including a microarray or multiwell plate); or any other agent known in the art.

Linking can be covalent or noncovalent, and can be direct or indirect. Methods for linking antibodies to such agents are well known in the art. See, e.g. Kennedy et al. (1976) Clin. Chim. Acta 70:1-31, and Schurs et al. (1977) Clin. Chim. Acta 81:1-40 (describing coupling techniques, including the glutaraldehyde method, the periodate method, the dimaleimide method, the m-maleimidobenzyl-N-hydroxy-succinimide ester method, each of which methods are incorporated by reference herein).

Methods for separating an antibody-protein complex from a sample are known in the art and include use of a capture agent that binds a binding partner (e.g., avidin to capture a biotin-linked antibody; an oligonucleotide to capture an oligonucleotide linked to an antibody; Physical separation can also be used, such as sedimentation, filtration, FACS (for example, using beads that are labeled with a spectral signature), and magnetic separation (when the antibody is linked to a matrix with magnetic properties, such as a magnetic bead).

Many binding partners are known in the art (e.g., a dinitrophenyl group, digoxigenin, fluorophores, Oregon Green dyes, Alexa Fluor 488 (Molecular Probes), fluorescein, a dansyl group, Marina Blue (Molecular Probes), tetramethylrhodamine, Texas Red (Molecular Probes), BODIPY (4,4-difluoro-4-bora-3a,4a-diaza-s-indacene; U.S. Pat. No. 4,774,339) dyes, etc) that can be used in the invention. Antibodies that can be used as capture reagents can specifically bind to binding agents are commercially available from vendors such as Molecular Probes, Eugene, Oreg. These antibodies include antibodies that can specifically bind to a dinitrophenyl group, a digoxigenin, a fluorophore, Oregon Green dyes, Alexa Fluor 488 (Molecular Probes), fluorescein, a dansyl group, Marina Blue (Molecular Probes), tetrahmethylrhodamine, Texas Red (Molecular Probes), and a BODIPY dye (Molecular Probes). Any suitable ligand and anti-ligand can also be used.

Oligonucleotides can be used as binding partner and capture reagents. Oligonucleotides include nucleic acids such as DNA, RNA, and mixed RNA/DNA molecules. The oligonucleotide that is used as the affinity label can hybridize to the sequence of the oligonucleotide on the capture reagent. Those of skill in the art will recognize that many different oligonucleotide sequences can be designed that will hybridize to each other. Important considerations for designing such oligonucleotide pairs include the actual nucleotide sequence, the length of the oligonucleotides, the hybridization conditions (e.g., temperature, salt concentration, presence of organic chemicals, etc.) and the melting temperature of the oligonucleotide.

Solid supports suitable for immobilizing (linking) antibodies or proteins from a sample (and modifications to render solid supports suitable for immobilizing antibodies) are well known in the art. Examples of a solid support include: a bead (including magnetized beads), microwell plate, and a protein microarray (See, e.g. U.S. Pat. No. 6,365,418). Thus, for example, CdSe-CdS core-shell nanocrystals enclosed in a silica shell can be easily derivatized for coupling to a biological molecule. Bruchez et al. (1998) Science 281:2013-2016. Similarly, highly fluorescent quantum dots (zinc sulfide-capped cadmium selenide) have been covalently coupled to biomolecules for use in ultrasensitive biological detection. Warren and Nie (1998) Science 281:2016-2018. Fluorescently labeled beads are commercially available from Luminex and Quantum Dot.

The bound protein (or in some embodiments, polypeptide fragments) can be released from the antibody-protein complex using conventional immunoaffinity elution conditions such as acidic pH, ionic strength, detergents or a combination thereof. In embodiments, peptide or protein is de-salted for subsequent fractionation, characterization, or other analysis.

In more than one embodiment, the antibody or the antigen can be immobilized, for example, to facilitate separation of complexed from uncomplexed forms of one or both. Observation of the antibody-antigen complex can be accomplished in any vessel suitable for containing the reactants. Examples of such vessels include microtiter plates, test tubes, and micro centrifuge tubes. In one embodiment, a fusion protein can be provided that adds a domain that allows one or both of the proteins to be bound to a matrix. For example, GST antibody fusion proteins or GST antigen fusion proteins can be adsorbed onto glutathione sepharose beads (Sigma Chemical, St. Louis, MO) or glutathione derivatized microtiter plates, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined directly or indirectly. Alternatively, the complexes can be dissociated from the matrix, and the level of antibody-antigen complex formation can be determined using standard techniques.

Other techniques for immobilizing proteins on matrices can also be used in the assays of the invention. For example, the antibody or the antigen (e.g. the 1593 peptide or 65D3-1 antibody) can be immobilized utilizing conjugation of biotin and streptavidin. Biotinylated antibody or antigen molecules can be prepared from biotin NHS (N hydroxy succinimide) using techniques well known within the art (e.g., biotinylation kit, Pierce Chemicals, Rockford, Ill.), and immobilized in the wells of streptavidin coated 96 well plates (Pierce Chemical). Alternatively, other antibodies reactive with the antibody or antigen of interest, but which do not interfere with the formation of the antibody-antigen complex of interest, can be derivatized to the wells of the plate, and unbound antibody or antigen trapped in the wells by antibody conjugation. Methods for detecting such complexes, in addition to those described herein for the GST immobilized complexes, include immunodetection of complexes using such other antibodies reactive with the antibody or antigen.

In some embodiments, the methods of the invention further comprise treating the sample with a protein cleaving agent, whereby polypeptide fragments are generated. In one embodiment, the sample is contacted with a protein cleaving agent prior to contacting a sample with an anti-iAP antibody. In another embodiment, protein is contacted with a protein cleaving agent after separation of protein from an antibody-protein complex.

Protein cleaving agent treatment generates protein cleavage fragments (such as polypeptides), which can facilitate subsequent mass spectral analysis of the amount of protein and the identity of proteins in a sample(s). Treatment with a protein cleaving agent treatment can facilitate the analysis of proteins whose molecular masses exceed 25 kDa. Protein cleaving reagent treatment also can facilitate accessibility and/or access of antibodies to a cognate epitope. Protein cleaving agents are well known in the art, and are further discussed herein. In some embodiments, one protein cleaving agent is used. In other embodiments, more than one protein cleaving reagent is used. In some embodiments, more than one type of protein cleaving agent is used with respect to a single sample (e.g., two or more types of proteases, two or more types of chemical cleavage agents, or a combination of one or more protease and one or more chemical cleavage agent). Conditions for treatment with a protein cleaving agent are well known in the art.

In one embodiment, a protein cleaving agent is a protease. Example of proteases that can be used as protein cleaving agents, include, but are not limited to: chymotrypsin, trypsin (arg, lys cleavage sequence), thermolysin (phe, leu, iso, val cleavage sequence), V8 protease, Endoproteinase Glu-C, Endoproteinase Asp-N, Endoproteinase Lys-C, Endoproteinase Arg-C, Endoproteinase Arg-N, Factor Xa protease, thrombin, enterokinase, V5 protease, and the tobacco etch virus protease. Proteases useful in the methods of the invention can be genetically engineered and/or chemically modified to prevent autolysis. It is appreciated that an enzymatic protein cleaving agent (such as a protease) can be modified to facilitate removal of the protease from the polypeptide cleavage products following polypeptide cleavage. Such modifications are known in the art and include: (1) bead-bound (e.g., latex, silica or magnetic bead) protease, (2) haptenated protease, (3) affinity depletion of the protease (with, for example, a bead-bound anti-protease, or bead-bound non-cleavable substrate) and/or (4) size exclusion chromatography. The activity of a protease can be inhibited, for example, by treating with heat, a protease inhibitor, a metal chelator (e.g., EGTA, EDTA), and the like.

In another embodiment, a protein cleaving agent is a chemical cleaving agent, such as chemical substances and compounds that cleave polypeptides and peptide bonds. Nonlimiting examples of chemical cleaving agents include cyanogen bromide (which cleaves at methionine residues), hydroxylamine (which cleaves between an Asn and a Gly residue), and acid pH (which can cleave an Asp-Pro bond) (see e.g., Ausubel et al., supra).

In still further embodiments, phosphatases (e.g., alkaline phosphatase, acid phosphatase, protein serine phosphatase, protein tyrosine phosphatase, protein threonine phosphatase, etc.), lipases, and other enzymes can be employed as protein cleaving agents.

In another aspect, the invention provides methods for characterizing a protein using mass spectrometry, comprising: (a) reducing the complexity of a sample using any of the methods described herein, whereby proteins are enriched and/or purified; and (b) analyzing the proteins (interchangeably termed “products”) which are isolated, purified, prepared and/or separated using any of the methods herein, wherein the analyzing is by mass spectrometry. Mass spectrometry methods are well known in the art. For example, in some embodiments, the mass spectrometry method is matrix-assisted laser desorption/ionization (“MALDI”) mass spectrometry; surface-enhanced laser desorption ionization (“SELDI”); and/or tandem mass spectrometry (e.g., MS/MS, MS/MS/MS, ESI-MS/MS). In some embodiments, tandem mass spectrometry is carried out using a laser desorption/ionization mass spectrometer that is further coupled to a quadrupole time-of-flight mass spectrometer QqTOF MS (see e.g., Krutchmsky et al, WO 99/38185). Methods such as MALDI-QqTOFMS (Krutchinsky et al., WO 99/38185; Shevchenko et al. (2000) Anal. Chem. 72:2132-2141), ESI-QqTOF MS (Figeys et al. (1998) Rapid Comm′ns. Mass Spec. 12-1435-144) and chip capillary electrophoresis (chip-CE)-QqTOF MS (Li et al. (2000) Anal. Chem. 72:599-609) have been described previously. Mass spectrometers and techniques for using them in methods of the invention are well known to those of skill in the art. A person skilled in the art understands that any of the components of a mass spectrometer (e.g., desorption source, mass analyzer, detect, etc.) can be combined with other suitable components described herein or those known in the art. For additional information regarding mass spectrometers, see, e.g., Principles of Instrumental Analysis, 3rd ed., Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

An antibody specific for iAP can be used to isolate an iAP polypeptide by standard techniques, such as immunoaffinity, chromatography or immunoprecipitation. Antibodies directed against an iAP protein (or a fragment thereof) can be used diagnostically to monitor protein levels in a biological sample, such as a tissue or fluid, as part of a clinical testing procedure, e.g., to, for example, determine the efficacy of a given treatment regimen or diagnose a given disease or infection.

In embodiments, detection can be facilitated by coupling (i.e., physically linking) the antibody to a detectable substance. Examples of detectable substances include those described herein.

Antibodies of the invention, including polyclonal, monoclonal, humanized and fully human antibodies, can be used as diagnostic agents. Such agents can be employed to diagnose an iAP-related disease or pathology in a subject.

An effective amount of an antibody of the invention can refer to an amount needed to achieve an objective. As noted herein, this can be due to a binding interaction between the antibody and its target antigen. The effective amount required will furthermore depend on the binding affinity of the antibody for its specific antigen.

An antibody according to the invention can be used as an agent for detecting the presence of iAP (or a protein or a protein fragment thereof) in a sample. For example, the antibody contains a detectable label. Antibodies can be polyclonal or monoclonal. In embodiments, the antibody is an intact antibody. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end labeling of a DNA probe with biotin such that it can be detected with fluorescently labeled streptavidin.

The detection method of the invention can be used to detect an analyte in a biological sample in vitro as well as in vivo. For example, in vitro techniques for detection of an analyte protein include enzyme linked immunosorbent assays (ELISAs), Western blots, immunoprecipitations, and immunofluorescence. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti analyte protein antibody. For example, the antibody can be labeled with a radioactive marker whose presence and location in a subject can be detected by standard imaging techniques.

Methods of Treatment of iAP-Associated Gastrointestinal Disease

As used herein, the terms “treat” or “treatment” can refer to both therapeutic treatment and prophylactic or preventative measures, wherein the object is to prevent or slow down (lessen) a physiological change or disorder, such as the progression of cancer. Clinical results can include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a gastrointestinal disease (for example, if an early detection gastrointestinal disease biomarker is identified in such a subject), or other cell proliferation-related diseases or disorders. Such diseases or disorders include but are not limited to, e.g., those diseases or disorders associated with aberrant expression of iAP. For example, the methods are used to treat, prevent or alleviate a symptom of gastrointestinal disease. In an embodiment, the methods are used to treat, prevent or alleviate a symptom of a gastrointestinal disease. Non-limiting examples of gastrointestinal diseases include colitis, inflammatory bowel disease (IBD), gastritis, gastroenteritis, pyloric stenosis, gastric cancer, infectious diarrhea, fecal impaction, constipation, intestinal obstruction and pseudoobstruction, or malabsorption. In addition to necrotizing enterocolitis (NEC), non-limiting examples of types of colitis comprise adult necrotizing enterocolitis (ANEC), pseudomembranous enterocolitis, infectious colitis, very early onset inflammatory bowel disease, ulcerative colitis, Crohn's disease, ischemic colitis, and radiation colitis.

Accordingly, in one aspect, the invention provides methods for preventing, treating or alleviating a symptom of a gastrointestinal disease in a subject by administering to the subject a monoclonal antibody, scFv antibody or bi-specific antibody of the invention. For example, an anti-iAP antibody can be administered in therapeutically effective amounts.

Subjects at risk for gastrointestinal diseases or disorders can include patients who have a family history of gastrointestinal disease or a subject exposed to a known or suspected gastrointestinal disease-causing agent. Administration of a prophylactic agent can occur prior to the manifestation of gastrointestinal disease such that the disease is prevented or, alternatively, delayed in its progression.

### Subjects

As described herein, embodiments of the invention comprise methods of diagnosing, prognosing, and/or treating a subject with a gastrointestinal disease. The term “subject” or “patient” can refer to any organism to which aspects of the invention can be administered, e.g., for experimental, diagnostic, prophylactic, and/or therapeutic purposes. Subjects to which compounds of the can be administered will be mammals, such as primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals such as pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted herein or another organism that is alive. The term “living subject” refers to the entire subject or organism and not just a part excised (e.g., a liver or other organ) from the living subject.

In embodiments herein, a subject comprises a mammal, such as a human or vertebrate animal. Examples of such include but are not limited to a dog, cat, horse, cow, pig, sheep, goat, chicken, primate, e.g., monkey, fish (aquaculture species), e.g. salmon, rat, and mouse. A human comprises a preterm neonate, an infant, a child, an adolescent, an adult, or an elderly individual.

Although aspects of the invention as described herein relate to human gastrointestinal disorders, aspects of the invention are also applicable to other nonhuman vertebrates. Aspects of the invention are applicable for veterinary use, such as with domestic animals. In general, aspects will vary according to the type of use and mode of administration, as well as the requirements of individual subjects.

In embodiments, the subject can be on an antibiotic regimen. The term “antibiotic regimen” refers to the treatment or prevention of a disease, such as an infection, or method for achieving a change, such as the reducing or prevention of an infection, wherein said treatment comprises administering to a subject an antibiotic to effectively treat the disease or produce the physiological change. An antibiotic regimen can comprise variations known to those skilled in the art, such as antibiotic choice (for example, comprises correct medication choice, route of administration and dosing schedule), timing of administration, and duration. Non-limiting examples of such antibiotics comprise Vancomycin, Ampicillin, Zosyn (combination of piperacillin and tazobactam), Gentamycin, Flagyl (metrodniazole generic), Meropenem, Metronidazole, Cefotaxime, Clindamycin, or any combination thereof. In some embodiments, an antifungal agent can further be administered. In other embodiments, the antifungal agent can be Fluconazole, Terconazole, Voriconazole, Posaconazole, Pentamidine, Itraconazole, Ketoconazole.

### Screening Assays and Methods

The invention provides screening assays and methods for identifying therapeutics and/or prophylactics, i.e., candidate or test compounds or agents (e.g., peptides, peptidomimetics, small molecules or other drugs) that can be used to prevent or treat an iAP-associated disorder. The invention also encompasses the candidates and/or compounds identified using the screening assays described herein.

The term “screening” can refer to determining whether a candidate and/or test compound has capabilities or characteristics of preventing or slowing down (lessening) the targeted pathologic condition stated herein. In some embodiments, the peptide antibodies described herein can be used in immunoassays to evaluate approaches that specifically inhibit the expression or secretion of iAP. For example, the antibody can be used in assays that utilize denatured protein (e.g. Western blots). In other embodiments, the peptide antibody described herein can be useful in preclinical studies to evaluate the efficacy of candidate drugs, since it can rapidly quantitate the level of iAP a sample. Without wishing to be bound by theory, the level of iAP can be serially analyzed during treatment in pre-clinical animal models. The ability to examine the longitudinal response to treatment in the same animal can be highly valuable. This ability can be very useful in studies designed to evaluate dose responses and treatment intervals.

Embodiments of the invention can comprise measuring or detecting such biomarkers of an iAP-associated disease using assays known to the art. Non-limiting examples of assays include an immunoassay, a colorimetric assay, fluorimetric assay or a combination thereof. Non-limiting examples of immunoassays comprise a western blot assay, an enzyme-linked immunosorbent assay (ELISA), immunoprecipitation or a combination thereof. For example, a biological sample collected from a subject can be incubated together with a biomarker specific antibody, such as an anti-iAP antibody or fragment thereof, and the binding of the antibody to the biomarker in the sample is detected or measured.

In embodiments, the assay can be a multiplex assay. A “multiplex assay” can refer to a type of procedure that simultaneously measures multiple analytes (dozens or more) in a single assay and is distinguished from procedures that measure one or a few analytes at a time. Multiplex assays can be used to detect or to assay a given class of molecules within a biological sample, such as to diagnose a disease or to determine the effect of a treatment.

The candidate and/or test compounds of the invention can be obtained using any of the numerous approaches in combinatorial library methods known in the art, including biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. (See, e.g., Lam, 1997. Anticancer Drug Design 12:145).

The methods described herein can involve obtaining a biological sample from the subject. As used herein, the phrase “obtaining a biological sample” can refer to any process for directly or indirectly acquiring a biological sample from a subject. For example, a biological sample can be obtained (e.g., at a point-of-care facility, such as a physician's office, a hospital, laboratory facility) by procuring a sample (e.g., tissue sample, fluid sample, or waste sample) from a subject. Alternatively, a biological sample can be obtained by receiving the biological sample (e.g., at a laboratory facility) from one or more persons who procured the sample directly from the subject. The biological sample can be, for example, feces, such as stool, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

A “small molecule” as used herein, is meant to refer to a composition that has a molecular weight of less than about 5 kDa and, for example, less than about 4 kDa. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90:6909; Erb, et al., 1994. Proc. Natl. Acad. Sci. U.S.A. 91: 11422; Zuckermann, et al., 1994. J. Med. Chem. 37:2678; Cho, et al., 1993. Science 261:1303; Carrell, et al., 1994. Angew. Chem. Int. Ed. Engl. 33:2059; Carell, et al., 1994. Angew. Chem. Int. Ed. Engl. 33:2061; and Gallop, et al., 1994. J. Med. Chem. 37:1233.

Libraries of compounds can be presented in solution (see e.g., Houghten, 1992. Biotechniques 13:412-421), or on beads (see Lam, 1991. Nature 354:82-84), on chips (see Fodor, 1993. Nature 364:555-556), bacteria (see U.S. Pat. No. 5,223,409), spores (see U.S. Pat. No. 5,233,409), plasmids (see Cull, et al., 1992. Proc. Natl. Acad. Sci. USA 89:1865-1869) or on phage (see Scott and Smith, 1990. Science 249:386-390; Devlin, 1990. Science 249:404-406; Cwirla, et al., 1990. Proc. Natl. Acad. Sci. U.S.A. 87:6378-6382; Felici, 1991. J. Mol. Biol. 222:301-310; and U.S. Pat. No. 5,233,409.).

Those skilled in the art will recognize that, in any of the screening methods disclosed herein, the antibody can be a iAP-specific antibody. Additionally, the antigen can be iAP protein, or a portion or fragment thereof.

The screening methods disclosed herein can be performed as a cell-based assay or as a cell-free assay. The cell-free assays of the invention are amenable to the use of both the soluble form or the membrane-bound form of the proteins and fragments thereof. In the case of cell-free assays comprising the membrane-bound forms of the proteins, a solubilizing agent can be used such that the membrane-bound form of the proteins are maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly (ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

The invention further pertains to new agents identified by any of the aforementioned screening assays and uses thereof for treatments as described herein.

### Diagnostic Assays

The anti-iAP antibodies can be used diagnostically to, for example, monitor the development or progression of gastrointestinal disease as part of a clinical testing procedure, e.g., to determine the efficacy of a given treatment and/or prevention regimen.

In some embodiments, for diagnostic purposes, the anti-iAP antibody of the invention is linked to a detectable moiety, for example, so as to provide a method for detecting gastrointestinal disease in a subject at risk of or suffering from a gastrointestinal disease.

The detectable moieties can be conjugated directly to the antibodies or fragments, or indirectly by using, for example, a fluorescent secondary antibody. Direct conjugation can be accomplished by standard chemical coupling of, for example, a fluorophore to the antibody or antibody fragment, or through genetic engineering. Chimeras, or fusion proteins can be constructed which contain an antibody or antibody fragment coupled to a fluorescent or bioluminescent protein. For example, Casadei, et al, (Proc Natl Acad Sci USA. 1990 March; 87 (6): 2047-51) describe a method of making a vector construct that can express a fusion protein of aequorin and an antibody gene in mammalian cells. For example, the detectable moiety can be Europium-labeled, SULFO-TAG-labeled, HRP, or Alexa Fluor.

As used herein, the term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject (such as a biopsy), as well as tissues, cells and fluids within a subject. That is, the detection method of the invention can be used to detect cells that express iAP in a biological sample in vitro as well as in vivo. For example, in vitro techniques for detection of iAP include enzyme linked immunosorbent assays (ELISAs), Western blots, immunoprecipitations, and immunofluorescence. Furthermore, in vivo techniques for detection of iAP include introducing into a subject a labeled anti-iAP antibody. For example, the antibody can be labeled with a radioactive marker whose presence and location in a subject can be detected by standard imaging techniques.

In the case of “targeted” conjugates, that is, conjugates which contain a targeting moiety—a molecule or feature designed to localize the conjugate within a subject or animal at a site or sites, localization can refer to a state when an equilibrium between bound, “localized”, and unbound, “free” entities within a subject has been essentially achieved. The rate at which such equilibrium is achieved depends upon the route of administration. For example, a conjugate administered by intravenous injection can achieve localization within minutes of injection. On the other hand, a conjugate administered orally can take hours to achieve localization. Alternatively, localization can refer to the location of the entity within the subject or animal at selected time periods after the entity is administered. By way of another example, localization is achieved when a moiety becomes distributed following administration.

A reasonable estimate of the time to achieve localization can be made by one skilled in the art. Furthermore, the state of localization as a function of time can be followed by imaging the detectable moiety (e.g., a light-emitting conjugate) according to the methods of the invention, such as with a photodetector device. The “photodetector device” used can have a high enough sensitivity to allow for the imaging of faint light from within a mammal in a reasonable amount of time, and to use the signal from such a device to construct an image.

In cases where light-generating moieties which are extremely bright can be used, and/or to detect light-generating fusion proteins localized near the surface of the subject or animal being imaged, a pair of “night-vision” goggles or a standard high-sensitivity video camera, such as a Silicon Intensified Tube (SIT) camera (e.g., from Hammamatsu Photonic Systems, Bridgewater, N.J.), can be used. In embodiments, a more sensitive method of light detection can be used.

In extremely low light levels the photon flux per unit area becomes so low that the scene being imaged no longer appears continuous. Instead, it is represented by individual photons which are both temporally and spatially distinct form one another. Viewed on a monitor, such an image appears as scintillating points of light, each representing a single detected photon. By accumulating these detected photons in a digital image processor over time, an image can be acquired and constructed. In contrast to conventional cameras where the signal at each image point is assigned an intensity value, in photon counting imaging the amplitude of the signal carries no significance. The objective is to detect the presence of a signal (photon) and to count the occurrence of the signal with respect to its position over time.

Photodetector devices, such as those described herein, can detect individual photons and generate a signal which can be analyzed by an image processor. Reduced-Noise Photodetection devices achieve sensitivity by reducing the background noise in the photon detector, as opposed to amplifying the photon signal. Noise is reduced primarily by cooling the detector array. The devices include charge coupled device (CCD) cameras referred to as “backthinned”, cooled CCD cameras. In the more sensitive instruments, the cooling is achieved using, for example, liquid nitrogen, which brings the temperature of the CCD array to approximately −120° C. “Backthinned” refers to an ultra-thin backplate that reduces the path length that a photon follows to be detected, thereby increasing the quantum efficiency. A sensitive backthinned cryogenic CCD camera is the “TECH 512”, a series 200 camera available from Photometries, Ltd. (Tucson, Ariz.).

“Photon amplification devices” amplify photons before they hit the detection screen. This class includes CCD cameras with intensifiers, such as microchannel intensifiers. A microchannel intensifier contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image processors process signals generated by photodetector devices which count photons in order to construct an image which can be, for example, displayed on a monitor or printed on a video printer. Such image processors are can be sold as part of systems which include the sensitive photon-counting cameras described herein, and accordingly, are available from the same sources. The image processors can be connected to a personal computer, such as an IBM-compatible PC or an Apple Macintosh (Apple Computer, Cupertino, Calif), which can or cannot be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

In an embodiment, the biological sample contains protein molecules from the test subject. One exemplary biological sample is a fecal sample isolated by conventional means from a subject.

The invention also encompasses kits for detecting the presence of iAP or an iAP-expressing cell in a biological sample. For example, the kit can comprise: a labeled compound or agent that can detect a gastrointestinal disease (e.g., an anti-iAP scFv or monoclonal antibody) in a biological sample; means for determining the amount of iAP in the sample; and means for comparing the amount of iAP in the sample with a standard. The compound or agent can be packaged in a suitable container. The kit can further comprise instructions for using the kit to detect biomarkers of gastrointestinal disease in a sample.

In embodiments, the diagnostic assay comprises a disposable article for detecting or measuring biomarkers of gastrointestinal diseases. The disposable article can comprise a biosensor, and can optionally comprise other components known to the art. In embodiments, the biosensor can comprise at least one bio-recognition element.

In embodiments, the biosensor can detect or measure iAP in a sample. In other embodiments, the biosensor can detect or measure iAP enzymatic activity, total fecal protein, iAP dimerization/dissociation, post-translationally modified iAP, or a combination thereof. Non-limiting examples of post-translational modifications and samples are described herein.

In embodiments, the biosensor can be an immunosensor, and can further comprise a detection signal. Non-limiting examples of detection signals comprise a radioactive signal, colorimetric signal, a fluorescent signal, chemiluminescent signal, or a combination thereof. For example, the biosensor can produce a new color or change in spectral absorption. In embodiments, the biosensor of the invention comprises a bio-recognition element, or molecular recognition element, that provides the highly specific binding or detection selectivity for an analyte, such as iAP. The bio-recognition element, or system, can be a biologically derived material such as an enzyme or sequence of enzymes; an antibody or fragment thereof; a membrane receptor protein; DNA; an organelle, a natural or synthetic cell membrane; an intact or partial viable or nonviable bacterial, plant or animal cell; or a piece of plant or mammalian tissues, and functions to interact specifically with a target biological analyte. The bio-recognition element is responsible for the selective recognition of the analyte and the physico-chemical signal that provides the basis for the output signal. The physico-chemical signal generated by the bio-recognition element or elements can be communicated visually to the wearer or caretaker (i.e., via a color change visible to the human eye). Other embodiments can produce optical signals, which can require other instrumentation to enhance the signal. These include fluorescence, bioluminescence, total internal reflectance resonance, surface plasmon resonance, Raman methods and other laser-based methods.

Alternatively, the signal can be processed via an associated transducer which, for example, can produce an electrical signal (e.g., current, potential, inductance, or impedance) that can be displayed (e.g., on a readout such as an LED or LCD display) or which triggers an audible or tactile (e.g., vibration) signal or which can trigger an actuator, as described herein. The signal can be qualitative (e.g., indicating the presence of the target biological analyte) or quantitative (i.e., a measurement of the amount or concentration of the target biological analyte). In such embodiments, the transducer can optionally produce an optical, thermal or acoustic signal.

In any case, the signal can also be durable (i.e., stable and readable over a length of time can be at least of the same magnitude as the usage life of the article) or transient (i.e., registering a real-time measurement). Additionally, the signal can be transmitted to a remote indicator site (e.g., via a wire, or transmitter, such as an infrared or rf transmitter) including other locations within or on the article or remote devices. Further, the biosensor, or any of its components, can be adapted to detect and/or signal only concentrations of the target biological analyte above a predefined threshold level (e.g., in cases wherein the target biological analyte is normally in the bodily waste or when the concentration of the analyte is below a known “danger” level).

In an embodiment, the disposable article can be a diaper to be worn by a subject. Non-limiting examples of additional disposable articles include wipe for cleaning a subject, dipstick, spoon, scoopula, filter paper, or swab.

In aspects of the invention, the disposable article as described herein can be a component of a kit useful for diagnosing a subject with a gastrointestinal disease. Additional components of kits of the invention can comprise a bio-recognition element, a support structure, and instructions for use thereof. For example, an iAP bio-recognition element, such as an antibody as described herein, can be immobilized to a solid support structure.

Non-limiting examples of the composition of the solid support structure comprise plastic, cardboard, glass, plexiglass, tin, paper, or a combination thereof. The solid support can also comprise a dip stick, spoon, scoopula, filter paper or swab.

### Kits

Aspects of the invention are further directed to a diagnostic kit of molecular biomarkers for identifying a subject exhibiting or having a predisposition to develop a gastrointestinal disease. In embodiments, the kit comprises at least one of a means for determining total fecal protein concentration, a means for determining intestinal alkaline phosphatase (iAP) activity, and an iAP bio-recognition element, wherein together represent a molecular signature that is indicative of the presence of or a predisposition to development of a gastrointestinal disease in a human subject. In embodiments, the signature comprises total protein concentration at least two standard deviations above the mean of a control sample, intestinal alkaline phosphatase protein concentration at least two standard deviations above the mean of a control sample, or intestinal alkaline phosphatase activity at least two standard deviations below the mean of a control sample. In still other embodiments, the signature is selected from at least two of the group comprising total protein concentration at least two standard deviations above the mean of a control sample, intestinal alkaline phosphatase protein concentration at least two standard deviations above the mean of a control sample, and intestinal alkaline phosphatase activity at least two standard deviations below the mean of a control sample. In embodiments, the control sample can comprise two or more control samples.

In one embodiment, the kit includes (a) a container that contains components and support structures as described herein, and optionally (b) informational material. The informational material can be descriptive, instructional, marketing or other material comprising the methods described herein and/or the use of the agents for diagnostic purposes. In an embodiment, the kit includes also includes a therapeutics, such as antibiotics, probiotics, or an iAP replacement composition.

The informational material of the kits is not limited in its form. In one embodiment, the informational material can include information about production of the components of the kit, such as molecular weight, concentration, date of expiration, batch or production site information, and so forth. In one embodiment, the informational material can comprise methods of using the components of the kit, (e.g., to diagnose a subject with a GI disorder). The information can be provided in a variety of formats, include printed text, computer readable material, video recording, or audio recording, or information that provides a link or address to substantive material.

The kit can include other ingredients, such as solvents or buffers, a stabilizer, or a preservative. Optionally, the kit can comprise therapeutic agents, such as iAP replacement compositions or antibiotics, that can be provided in any form, e.g., liquid, dried or lyophilized form, such as substantially pure and/or sterile. When the agents are provided in a liquid solution, the liquid solution can be an aqueous solution. When the agents are provided as a dried form, reconstitution can be by the addition of a suitable solvent. The solvent, e.g., sterile water or buffer, can optionally be provided in the kit. In one embodiment, the kit includes other ingredients for detecting the presence of iAP or an iAP-expressing cell in a biological sample. For example, the kit can comprise: a labeled compound or agent that can detect a gastrointestinal disease (e.g., an anti-iAP scFv or monoclonal antibody) in a biological sample; means for determining the amount of iAP in the sample; and means for comparing the amount of iAP in the sample with a standard. The compound or agent can be packaged in a suitable container. The kit can further comprise instructions for using the kit to detect biomarkers of gastrointestinal disease in a sample.

In embodiments, the kit can comprise a means for obtaining a sample from a subject and/or a vessel for storing the collected sample for a period of time; an agent that can detect iAP (e.g., an anti-iAP monoclonal antibody) in a sample; means for determining the amount of iAP in the sample; and means for comparing the amount of iAP in the sample with a standard. The compound or agent can be packaged in a suitable container. The kit can further comprise instructions for using the kit to detect iAP in a sample.

In an embodiment, the kit can be used in conjunction with capital equipment (such as a mass spectrometer) for the detection of iAP. The assay kit can include reagents for use in sample preparation. These reagents can include commercially available materials such as, but not limited to nanoparticles, digestion compounds, and wash buffers for rinsing. Other components within the kit can include pipette tips, aliquot tubes (sometimes referred to as ‘vials’ or ‘Eppendorf tubes’), an aliquot stand for tube management, sample identification labeling, and device labelling, such as Instructions for Use (IFU). The kit can further consist of reusable capital components including a magnet for separation of nanoparticles and/or additional disposable components.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention is further described in the following examples, which do not limit the scope of the invention described in the claims.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Example 1: CHOSEN DIAGNOSTIC INC MONOCLONAL ANTIBODY IP

A recombinant monoclonal antibody with the following properties was generated using a peptide synthesized from the human sequence of intestinal alkaline phosphatase.

Human intestinal alkaline phosphatase epitope:
(SEQ ID NO: 11)
PEYPADASQNGIRLDGK
>CDI005E8_H (heavy chain)
(SEQ ID NO: 9)
ATGGAGACTGGGCTGCGCTGGCTTCTCCTGGTCGCTGTGCTCAAAGGTGT
CCAGTGTCAGTCGTTGGAGGAGTCCGGGGGAGGCCTGGTCCAGCCTGGGG
CATCCCTGACACTCACCTGCACAGCCTCTGGATTCTCCTTCACTAACAAC
TATTGGATATATTGGGTCCGCCAGGCTCCAGGGAAGGGGCTGGAGTGGAT
CGGATCCGTGATTATTAGTAGTAGTAGCGCATACTACGCGAGCTGGGCGA
AAGGCCGATTCACCATCACCAAACCCTCGTCGACCACGGTGACTCTGCAG
GTGACCAGTCTGACAGCCGCGGACACGGCCACCTATTTCTGTGTGACAGG
TGGTATCTTATGGGGCCCAGGCACCCTGGTCACCGTCTCCTCAGGGCAAC
CTAAGGCTCCATCAGTCTTCCCACTGGCCCCCTGCTGCGGGGACACACCC
AGCTCCACGGTGACCCTGGGCTGCCTGGTCAAAGGCTACCTCCCGGAGCC
AGTGACCGTGACCTGGAACTCGGGCACCCTCACCAATGGGGTACGCACCT
TCCCGTCCGTCCGGCAGTCCTCAGGCCTCTACTCGCTGAGCAGCGTGGTG
AGCGTGACCTCAAGCAGCCAGCCCGTCACCTGCAACGTGGCCCACCCAGC
CACCAACACCAAAGTGGACAAGACCGTTGCGCCCTCGACATGCAGCAAGC
CCATGTGCCCACCCCCTGAACTCCCGGGGGGACCGTCTGTCTTCATCTTC
CCCCCAAAACCCAAGGACACCCTCATGATCTCACGCACCCCCGAGGTCAC
ATGCGTGGTGGTGGACGTGAGCCAGGATGACCCCGAGGTGCAGTTCACAT
GGTACATAAACAACGAGCAGGTGCGCACCGCCCGGCCGCCGCTACGGGAG
CAGCAGTTCAACAGCACGATCCGCGTGGTCAGCACCCTCCCCATCGCGCA
CCAGGACTGGCTGAGGGGCAAGGAGTTCAAGTGCAAAGTCCACAACAAGG
CACTCCCGGCCCCCATCGAGAAAACCATCTCCAAAGCCAGAGGGCAGCCC
CTGGAGCCGAAGGTCTACACCATGGGCCCTCCCCGGGAGGAGCTGAGCAG
CAGGTCGGTCAGCCTGACCTGCATGATCAACGGCTTCTACCCTTCCGACA
TCTCGGTGGAGTGGGAGAAGAACGGGAAGGCAGAGGACAACTACAAGACC
ACGCCGACCGTGCTGGACAGCGACGGCTCCTACTTCCTCTACAGCAAGCT
CTCAGTGCCCACGAGTGAGTGGCAGCGGGGCGACGTCTTCACCTGCTCCG
TGATGCACGAGGCCTTGCACAACCACTACACGCAGAAGTCCATCTCCCGC
TCTCCGGGTAAATAG
>CDI005E8_L (light chain)
(SEQ ID NO: 10)
ATGGACACGAGGGCCCCCACTCAGCTGCTGGGGCTCCTGCTGCTCTGGCT
CCCAGGTGCCAGATGTGATGTTGTGATGACCCAGACTCCAGCCTCCGTGG
AGGCAGCTGTGGGAGGCACAGTCACCATCAAGTGCCAGGCCAGTCAGAGC
ATTGGCACTGCATTAGCCTGGTATCAGCAGAAACCAGGGCAGCCTCCCAA
GCGCCTGATCTACAAGGCATCCACTCTGACATCTGGGGTCCCATCGCGGT
TCAAAGGCAGTGGATCTGGGACAGATTTCACTCTCACCATCAGCGACCTG
GAGTGTGCCGATGCTGCCACTTACTACTGTCAATGTACTGTTGATGATAG
TAGTAGTGGTAGTAGTTATGGTAATGCTTTCGGCGGAGGGACCGAGGTGG
TGGTCAGAGGTGATCCAGTTGCACCTACTGTCCTCATCTTCCCACCAGCT
GCTGATCAGGTGGCAACTGGAACAGTCACCATCGTGTGTGTGGCGAATAA
ATACTTTCCCGATGTCACCGTCACCTGGGAGGTGGATGGCACCACCCAAA
CAACTGGCATCGAGAACAGTAAAACACCGCAGAATTCTGCAGATTGTACC
TACAACCTCAGCAGCACTCTGACACTGACCAGCACACAGTACAACAGCCA
CAAAGAGTACACCTGCAAGGTGACCCAGGGCACGACCTCAGTCGTCCAGA
GCTTCAATAGGGGTGACTGTTAG
CDI005E8 Heavy chain
(SEQ ID NO: 1)
METGLRWLLLVAVLKGVQCQSLEESGGGLVQPGASLTLTCTASGFSFTNN
YWIYWVRQAPGKGLEWIGSVIISSSSAYYASWAKGRFTITKPSSTTVTLQ
VTSLTAADTATYFCVTGGILWGPGTLVTVSSGQPKAPSVFPLAPCCGDTP
SSTVTLGCLVKGYLPEPVTVTWNSGTLINGVRTFPSVRQSSGLYSLSSVV
SVTSSSQPVTCNVAHPATNTKVDKTVAPSTCSKPMCPPPELPGGPSVFIF
PPKPKDTLMISRTPEVTCVVVDVSQDDPEVQFTWYINNEQVRTARPPLRE
QQENSTIRVVSTLPIAHQDWLRGKEFKCKVHNKALPAPIEKTISKARGQP
LEPKVYTMGPPREELSSRSVSLTCMINGFYPSDISVEWEKNGKAEDNYKT
TPTVLDSDGSYFLYSKLSVPTSEWQRGDVFTCSVMHEALHNHYTQKSISR
SPGK
CDI005E8 Light chain
(SEQ ID NO: 2)
MDTRAPTQLLGLLLLWLPGARCDVVMTQTPASVEAAVGGTVTIKCQASQS
IGTALAWYQQKPGQPPKRLIYKASTLTSGVPSRFKGSGSGTDFTLTISDL
ECADAATYYCQCTVDDSSSGSSYGNAFGGGTEVVVRGDPVAPTVLIFPPA
ADQVATGTVTIVCVANKYFPDVTVTWEVDGTTQTTGIENSKTPQNSADCT
YNLSSTLTLTSTQYNSHKEYTCKVTQGTTSVVQSFNRGDC
CDI005E8 Heavy chain CDR1
(SEQ ID NO: 3)
CTASGFSFTNNYWIYWV
CDI005E8 Heavy chain CDR2
(SEQ ID NO: 4)
LEWIGSVIISSSSAYYASWAKGRFT
CDI005E8 Heavy chain CDR3
(SEQ ID NO: 5)
CVTGGILWGPGT
CDI005E8 Light chain CDR1
(SEQ ID NO: 6)
CQASQSIGTALAWYQ
CDI005E8 Light chain CDR1
(SEQ ID NO: 7)
IYKASTLTS
CDI005E8 Light chain CDR1
(SEQ ID NO: 8)
CTVDDSSSGSSYGNAFGGG

### Example 2

### Antibody Development File

Peptide antigen:
2-hsALPI=PEYPADASONGIRLDGK (SEQ ID NO: 11; human intestinal alkaline phosphatase protein aa234-aa250);
calculated MW=1830.9725 g/mol
Chain identification

Heavy chain antibody sequence of
CDI005E8 (SEQ ID NO: 1):
METGLRWLLLVAVLKGVQCQSLEESGGGLVQPGASLTLTCTASGFSFTNN
YWIYWVRQAPGKGLEWIGSVIISSSSAYYASWAKGRFTITKPSSTTVTLQ
VTSLTAADTATYFCVTGGILWGPGTLVTVSSGQPKAPSVFPLAPCCGDTP
SSTVTLGCLVKGYLPEPVTVTWNSGTLTNGVRTFPSVRQSSGLYSLSSVV
SVTSSSQPVTCNVAHPATNTKVDKTVAPSTCSKPMCPPPELPGGPSVFIF
PPKPKDTLMISRTPEVTCVVVDVSQDDPEVQFTWYINNEQVRTARPPLRE
QQFNSTIRVVSTLPIAHQDWLRGKEFKCKVHNKALPAPIEKTISKARGQP
LEPKVYTMGPPREELSSRSVSLTCMINGFYPSDISVEWEKNGKAEDNYKT
TPTVLDSDGSYFLYSKLSVPTSEWQRGDVFTCSVMHEALHNHYTQKSISR
SPGK
Light chain antibody sequence of
CDI005E8 (SEQ ID NO: 2):
MDTRAPTQLLGLLLLWLPGARCDVVMTQTPASVEAAVGGTVTIKCQASQS
IGTALAWYQQKPGQPPKRLIYKASTLTSGVPSRFKGSGSGTDFTLTISDL
ECADAATYYCQCTVDDSSSGSSYGNAFGGGTEVVVRGDPVAPTVLIFPPA
ADQVATGTVTIVCVANKYFPDVTVTWEVDGTTQTTGIENSKTPQNSADCT
YNLSSTLTLTSTQYNSHKEYTCKVTQGTTSVVQSENRGDC

Affinity and specificity determination:

K_{d}, CDI005E8 Ab=7.11±1.01 μM, when using 2 μg/mL (1.1 μM) of peptide antigen 2-hsALPI, which was surprising considering the hydrophobic iAP epitope and the hydrophobic antibody paratope are an unusual combination to elicit picomolar binding affinities.

CDI005E8 is specific for intestinal isoform and does not bind to purified placenta, tissue-nonspecific, or germ cell AP.

### Complementarity-Determining Region (CDR) Identification

Table 4. Without wishing to be bound by theory, key regions and residues in the CDI005E8 antigen binding fragment. To identify CDR regions, two different methods were used to analyze the sequences of the CDI005E8 heavy (VH) and light (VL) chains of the variable region (Fv): (i) Genscript website guidance or (ii) ANARCI calculations. For ANARCI calculations on the SAbPred server, PDB 5v6m was used for VH and VL frameworks. READ was used with CDR specific databases to predict the CDR conformations. If a knowledge-based prediction cannot be used, then MODELLER was used to predict the CDR loop. The following templates, methods, and scores were obtained: CDR H1: PDB ID 6cjk (D), fread score=71; CDR H2: PDB ID 2xlo (B); fread score=30; CDR H3: PDB ID 4okv (C), sequence similarity score=9; CDR L1: PDB ID 5vkd (L); fread score=44; CDR L2: PDB ID 5v6m (L), fread score=43; and CDR L3: PDB ID 6cjk (C), modeler ab initio score=25. The amino acid sequence common to both manual determination and ANARCI server calculations are underlined. Paratope, or antigen-binding region, was calculated using i-Patch algorithm; top 10 paratope residues in the VH and VL chains are shown, alongside their scores. Score describes how often the residue type in its local environment (patch) is involved in antigen binding in known structures. Paratope residues with highest four scores are shown in bold in the CDR prediction; paratope residue with highest score is in bold and in grey highlight. Abbreviations: Ka=Kabat; Ch=Chothia; IM=IMGT; No=North.

| CDR PREDICTION | | | | | | |
|---|---|---|---|---|---|---|
| | manual determination using | | | | PARATROPE | |
| Fv* | Genscript guidelines | ANARCI server | | | IDENTIFICATION** | |
| region | (Kabat or Chothia) | amino acid sequence | residue # | ID | residue | score |
| H1-CDR | 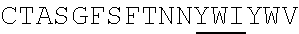 | 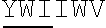 | 35-42 | Ka | 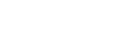 | 10.01 |
| | | 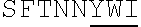 | 29-39 | Ch | VH-S64 | 6.43 |
| | | 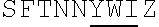 | 29-40 | IM | VA-706 | 5.49 |
| | | 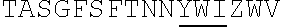 | 24-42 | No | VH-A68 | 5.43 |
| | | | | | VH-K72 | 4.93 |
| H2-CDR | 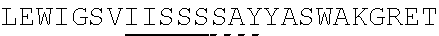 | 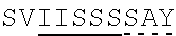 | 55-66 | Ka | VH-S69 | 4.69 |
| | | 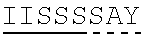 | 57-66 | Ch | VH-Y37 | 4.44 |
| | | 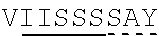 | 56-66 | IM | VH-T31 | 4.20 |
| | | 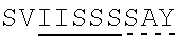 | 55-66 | No | VH-B76 | 4.13 |
| | | | | | VH-F72 | 4.05 |
| H3-CDR | CVTGGILWGPGT | GGIL | 107-117 | Ka | | |
| | | GGIL | 107-117 | Ch | | |
| | | VTGGIL | 105-117 | IM | | |
| | | VTGGIL | 105-117 | No | | |
| L1-CDR | CQASQSIGTALAWYQ | QASQSIGTALA | 24-40 | Ka | 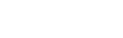 | 3.71 |
| | | QASQSIGTALA | 24-40 | Ch | VL-K31 | 3.56 |
| | | CQASQSIGTALA | 23-40 | IM | VL-755 | 3.41 |
| | | QASQSIGTALA | 24-40 | No | VL-0109 | 2.73 |
| | | | | | VL-A117 | 2.36 |
| L2-CDR | 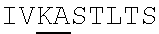 | 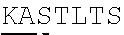 | 56-69 | Ka | VL-A12 | 2.09 |
| | | 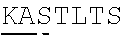 | 56-69 | Ch | VA-S65 | 2.02 |
| | | 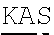 | 56-65 | IM | VL-T66 | 2.02 |
| | | 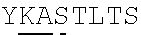 | 55-69 | No | VL-B107 | 1.97 |
| | | | | | VL-F111 | 1.95 |
| L3-CDR | 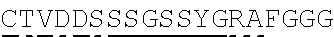 | 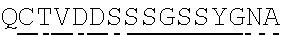 | 105-117 | Ka | | |
| | | 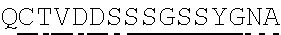 | 105-117 | Ch | | |
| | | 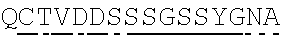 | 105-117 | IM | | |
| | | 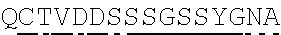 | 105-117 | No | | |

*H3-CDR can be the antibody molecule's most important component in conferring binding activity and specificity. This specific target binding is an outcome of unique V and D rearrangements and VL pairing; the HCDR3 is necessary, albeit insufficient, for soecific antibody binding.

EpiPred uses geometric matching of the antibody and antigen interfaces coupled with antibody-antigen-specific knowledge-based potential. Computational B-cell epitope prediction provides information about the regions of the antigen bound by the antibody, but it does not directly contribute to the knowledge of the antibody residues that need to be mutated so as to modify its function. This problem can be approached by antibody-antigen docking, which can provide a list of putative orientations of the two molecules with respect to each other. Antibody-antigen docking requires different methodology from that used for the corresponding problem in non-antibody targets. This is in part because antibodies use different residues in their binding sites when compared with both general proteins and antigens and thus an asymmetric scoring system is required that accounts for these discrepancies. Use of EpiPred was shown to enrich the top decoys with more native poses.

PROTEIN SEQUENCE OF HUMAN INTESTINAL ALKALINE PHOSPHATASE: UniProt P09923; NCBI Reference Sequence: NP_001622.2 intestinal-type alkaline phosphatase precursor [Homo sapiens] and NCBI Reference Sequence: NM 001631.5

### Structure of Human Intestinal Alkaline Phosphatase

Human intestinal alkaline phosphatase-no experimental structures available in the Protein Databank (PDB)

Assumption: a single static x-ray structure is sufficient to identify determinants of antibody-antigen recognition.

CDRs are part of the variable chains in immunoglobulins (antibodies) and T-cell receptors, generated by B-cells and T-cells respectively, where these molecules bind to their specific antigen. Exact identification of CDRs is crucial for understanding and manipulating antigenic interactions. One way to do this is by marking residues on the antibody that interact with B cell epitopes on the antigen. This requires identification of B cell epitopes and CDRs. Sequence and structural diversity are concentrated in six hypervariable loops or CDRs. A set of CDRs constitutes a paratope. Existing tools for identifying CDRs are based on sequence analysis or general biophysical principles. Two different methods were used here to identify CDR regions: (i) Genscript website guidance or (ii) ANARCI server.

For the Antigen receptor Numbering And Receptor Classification (ANARCI) software, this tool will provide numbering of amino acid sequence of antibody and T-cell receptor variable domains. ANACI aligns a given sequence to a database of Hidden Markov models that describe the germline sequences of antibody and TCR domain types. The most significant alignment predicts the domain type and species of the input sequence. Identification of domains and their annotation can be performed with IMGT, Chothia, Kabat, Martin (Enhanced Chothia) or AHo numbering schemes. Full description of the pipeline is published [Dunbar J and Deane CM. ANARCI: Antigen receptor numbering and receptor classification. Bioniformatics (2016)). Alternatively, web-interface program can be found at opig.stats.ox.ac.uk/webapps/anarci.

### Peptide Antigen Determination

### SUMMARY

Epitope discovery was performed using Discotope 2.0 and Discotope 1.0 of human intestinal alkaline phosphatase residues. Discotope 1.0 has a specificity of 75% and a sensitivity of 47.2% with the use of a −7.7 score threshold. Discotope 2.0 displayed improved performance both in cross-validation and in independent evaluations; it had a sensitivity of 82% with use of a −3.7 score threshold for a 15 aa peptide.

### Second Round

A candidate human iAP peptide antigen (P09923 (PPBI_HUMAN) was identified using DiscoTope 2.0.
aa234-250 17-mer, PEYPADASQNGIRLDGK (SEQ ID NO: 11) between α11 and α12

### First Round

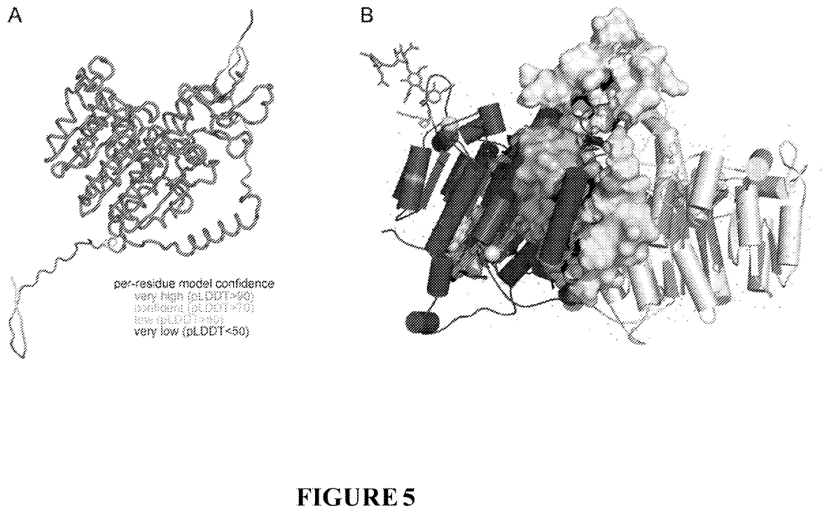

A candidate human iAP peptide antigen (P09923 (PPBI_HUMAN) was identified using DiscoTope 1.0 [FIG. 5].
aa234-250 17-mer, PEYPADASQNGIRLDGK (SEQ ID NO: 11) between all and a12 (SEQ ID NO: 18)

DiscoTope is a method for predicting discontinuous B-cell epitopes epitopes from 3D structures of proteins in PDB format.

https://services.healthtech.dtu.dk/service.php?DiscoTope-2.0

Reliable B Cell Epitope Predictions: Impacts of Method Development and Improved Benchmarking Jens Vindahl Kringelum, Claus Lundegaard, Ole Lund, and Morten Nielsen. Plos Computational Biology, 2012

Method utilizes calculation of surface accessibility (estimated in terms of contact numbers) and a new epitope propensity amino acid score. Final scores are calculated by combining the propensity scores of residues in spatial proximity and the contact numbers.

TABLE 5
Discotope 2.0 scores for human intestinal alkaline phosphatase:

| residue | residue | contact | propensity | discotope |
|---|---|---|---|---|
| number | name | number | score | score |
| 1 | MET | 0 | 1.014 | 0.898 |
| 2 | GLN | 0 | 0.355 | 0.314 |
| 3 | GLY | 2 | −1.183 | −1.277 |
| $ | PRO | 4 | −2.341 | −2.532 |
| 5 | TRP | 2 | −4.252 | −3.993 |
| 6 | VAL | 3 | −5.828 | −5.503 |
| 7 | LEU | 2 | −6.788 | −6.238 |
| 8 | LEU | 0 | −7.39 | −6.54 |
| 9 | LEU | 0 | −8.367 | −7.405 |
| 10 | LEU | 2 | −8.934 | −8.137 |
| 11 | GLY | 3 | −8.794 | −8.128 |
| 12 | LEU | 2 | −7.168 | −6.574 |
| 13 | ARG | 0 | −7.975 | −7.058 |
| 14 | LEU | 4 | −9.417 | −8.794 |
| 15 | GLN | 13 | −7.994 | −8.57 |
| 16 | LEU | 3 | −7.885 | −7.323 |
| 17 | SER | 15 | −7.844 | −8.667 |
| 18 | LEU | 7 | −7.526 | −7.466 |
| 19 | GLY | 13 | −7.562 | −8.187 |
| 20 | VAL | 16 | −4.75 | −6.044 |
| 21 | ILE | 4 | −3.708 | −3.742 |
| 22 | PRO | 17 | −2.55 | −4.212 |
| 23 | ALA | 0 | −1.049 | −0.928 |
| 24 | GLU | 8 | −0.446 | −1.814 |
| 25 | GLU | 12 | −1.242 | −2.479 |
| 26 | GLU | 5 | −0.562 | −1.077 |
| 27 | ASN | 7 | −0.554 | −1.295 |
| 28 | PRO | 0 | 0.387 | −0.873 |
| 29 | ALA | 3 | 0.955 | −3.39 |
| 30 | PHE | 8 | −0.325 | −1.211 |
| 31 | TRP | 7 | −1.198 | −1.866 |
| 32 | ASA | 4 | −1.34 | −1.646 |
| 33 | ARG | 2 | −1.585 | −2.208 |
| 34 | GUN | 11 | −1.637 | −2.714 |
| 35 | ALA | 2 | −2.938 | −3.40 |
| 36 | ALA | 6 | −2.183 | −2.62 |
| 37 | GUL | 7 | −2.503 | −3.02 |
| 38 | ALA | 12 | −6.712 | −7.32 |
| 39 | LEU | 6 | −5.453 | −5.516 |
| 40 | ASP | 6 | −3.626 | −3.899 |
| 41 | ALA | 12 | −6.426 | −7.067 |
| 42 | ALA | 8 | −8.583 | −8.516 |
| 43 | LYS | 6 | −4.962 | −5.081 |
| 44 | LYS | 8 | −6.435 | −6.615 |
| 45 | LEU | 21 | −12.243 | −13.25 |
| 46 | GLN | 5 | −13.767 | 12.759 |
| 47 | PRO | 11 | −19.048 | −18.122 |
| 48 | ILE | 30 | −20.632 | 21.709 |
| 49 | GLN | 3 | −18.409 | −16.637 |
| 50 | LYS | 21 | −20.695 | −20.73 |
| 51 | VAL | 24 | −25.434 | −25.269 |
| 52 | ALA | 38 | −31.769 | −32.486 |
| 53 | LYS | 19 | −28.884 | −27.747 |
| 54 | ASN | 32 | −36.129 | −35.654 |
| 55 | LEU | 33 | −43.655 | −42.43 |
| 56 | ILE | 41 | −46.249 | −45.645 |
| 57 | LEU | 41 | −46.015 | −45.438 |
| 58 | PHE | 40 | −42.759 | −42.442 |
| 59 | LEU | 43 | −41.223 | −41.428 |
| 60 | GLY | 46 | −32.767 | −34.289 |
| 61 | ASP | 39 | −30.256 | −31.262 |
| 62 | GLY | 35 | −23.686 | −24.987 |
| 63 | LEU | 36 | −26.727 | −27.793 |
| 64 | GLY | 30 | −25.385 | −25.915 |
| 65 | VAL | 13 | 22.884 | −21.747 |
| 66 | PRO | 26 | −17.669 | −18.627 |
| 67 | THR | 49 | −18.95 | −22.406 |
| 68 | VAL | 29 | −23.117 | −23.793 |
| 69 | THR | 15 | −18.787 | −18.352 |
| 70 | ALA | 38 | −17.084 | −19.49 |
| 71 | THR | 37 | −15.208 | −17.714 |
| 72 | ARG | 18 | −17.426 | −17.492 |
| 73 | ILE | 25 | −15.561 | −16.647 |
| 74 | LEU | 29 | −12.534 | −14.428 |
| 75 | LYS | 26 | −11.556 | −13.217 |
| 76 | GLY | 22 | 10.89 | −12.168 |
| 77 | GLN | 24 | −7.919 | −9.768 |
| 78 | LYS | 20 | −5.477 | −7.147 |
| 79 | ASN | 19 | −5.192 | −6.78 |
| 80 | GLY | 0 | −4.501 | −3.983 |
| 81 | LYS | 16 | −5.645 | −6.836 |
| 82 | LEU | 15 | −8.105 | −8.898 |
| 83 | GLY | 22 | −10.83 | −12.115 |
| 84 | PRO | 19 | −15.037 | −15.492 |
| 85 | GLU | 16 | −11.663 | −12.162 |
| 86 | THR | 21 | −13.9 | −14.717 |
| 87 | PRO | 11 | 16.52 | −15.885 |
| 88 | LEU | 36 | −21.814 | −23.445 |
| 89 | ALA | 24 | −24.284 | −24.252 |
| 90 | MET | 38 | −28.583 | −29.666 |
| 91 | ASP | 20 | −22.725 | −22.412 |
| 92 | ARG | 13 | −21.142 | −20.206 |
| 93 | PHE | 36 | 26.527 | −27.617 |
| 94 | PRO | 24 | −21.113 | −21.445 |
| 95 | TYR | 24 | −28.569 | −28.043 |
| 96 | LEU | 13 | −28.421 | −26.648 |
| 97 | ALA | 28 | −29.833 | −29.622 |
| 98 | LEU | 10 | −27.225 | −25.244 |
| 99 | SER | 43 | −28.886 | −30.509 |
| 100 | LYS | 14 | −22.704 | −21.703 |
| 101 | THR | 31 | −23.732 | −24.568 |
| 102 | TYR | 9 | −15.531 | −14.78 |
| 103 | ASN | 30 | −14.052 | −15.886 |
| 104 | VAL | 23 | −11.384 | −12.72 |
| 105 | ASP | 19 | −6.464 | −7.906 |
| 106 | ARG | 23 | −7.34 | −9.141 |
| 107 | GLN | 2 | −7.526 | −6.89 |
| 108 | VAL | 13 | −11.004 | −11.234 |
| 109 | PRO | 39 | 16.713 | −19.276 |
| 110 | ASP | 22 | −17.168 | −17.723 |
| 111 | SER | 33 | −25.488 | −26.352 |
| 112 | ALA | 27 | −22.023 | −22.595 |
| 113 | ALA | 29 | −23.122 | −23.798 |
| 114 | THR | 38 | −29.878 | −30.812 |
| 115 | ALA | 39 | −36.386 | −36.687 |
| 116 | THR | 34 | −32.36 | −32.549 |
| 117 | ALA | 34 | −34.466 | −34.412 |
| 118 | TYR | 44 | −40.608 | −40.998 |
| 119 | LEU | 42 | −43.753 | −43.552 |
| 120 | CYS | 32 | −38.132 | −37.427 |
| 121 | GLY | 19 | −35.145 | −33.288 |
| 122 | VAL | 22 | −30.666 | −29.669 |
| 123 | LYS | 19 | −25.013 | −24.322 |
| 124 | ALA | 43 | −18.986 | −21.748 |
| 125 | ASN | 20 | −11.181 | −12.196 |
| 126 | PHE | 11 | −10.105 | −10.208 |
| 127 | GLN | 10 | −9.841 | −9.859 |
| 128 | THR | 34 | −12.208 | −14.714 |
| 129 | ILE | 28 | −19.991 | −20.912 |
| 130 | GLY | 34 | −25.139 | −26.158 |
| 131 | LEU | 32 | −17.959 | −19.574 |
| 132 | SER | 8 | −15.044 | −14.234 |
| 133 | ALA | 17 | −10.74 | −11.46 |
| 134 | ALA | 9 | −9.228 | −9.202 |
| 135 | ALA | 34 | −10.075 | −12.827 |
| 136 | ARG | 5 | −7.774 | −7.455 |
| 137 | PHE | 18 | −6.994 | −8.26 |
| 138 | ASN | 6 | −7.066 | −6.943 |
| 139 | GLN | 13 | −6.936 | −7.633 |
| 140 | CYS | 17 | −8.667 | −9.625 |
| 141 | ASN | 3 | −7.18 | −6.699 |
| 142 | THR | 17 | −8.464 | −9.446 |
| 143 | THR | 26 | −10.389 | −12.185 |
| 144 | ARG | 8 | −8.45 | −8.399 |
| 145 | GLY | 5 | −9.81 | −9.257 |
| 146 | ASN | 27 | −10.827 | −12.687 |
| 147 | GLU | 20 | −17.215 | −17.536 |
| 148 | VAL | 30 | −24.85 | −25.442 |
| 149 | ILE | 14 | −28.723 | −27.03 |
| 150 | SER | 40 | −37.688 | −37.954 |
| 151 | VAL | 40 | −41.242 | 41.099 |
| 152 | MET | 34 | −40.21 | −39.496 |
| 153 | ASN | 24 | −33.271 | −32.204 |
| 154 | ARG | 28 | −31.213 | −30.843 |
| 155 | ALA | 43 | −35.103 | 36.011 |
| 156 | LYS | 24 | −29.247 | 28.643 |
| 157 | GLN | 17 | −20.693 | −20.268 |
| 158 | ALA | 32 | −22.093 | −23.232 |
| 159 | GLY | 3 | −19.556 | −17.652 |
| 160 | LYS | 34 | −27.63 | −28.363 |
| 161 | SER | 24 | −30.807 | −30.024 |
| 162 | VAL | 28 | −35.829 | −34.929 |
| 163 | GLY | 39 | −36.587 | −36.865 |
| 164 | VAL | 37 | −37.108 | −37.095 |
| 165 | VAL | 39 | −33.394 | −34.039 |
| 166 | THR | 37 | −29.707 | −30.545 |
| 167 | THR | 38 | 21.288 | −23.21 |
| 168 | THR | 32 | −17.929 | −19.547 |
| 169 | ARG | 28 | −13.547 | −15.21 |
| 170 | VAL | 39 | −20.754 | −22.852 |
| 171 | GLN | 36 | −17.237 | −19.395 |
| 172 | HIS | 36 | −19.224 | −21.153 |
| 173 | ALA | 32 | −24.998 | −25.803 |
| 174 | SER | 37 | −34.547 | −34.829 |
| 175 | PRO | 42 | −33.433 | −34.418 |
| 176 | ALA | 36 | −28.52 | 29.381 |
| 177 | GLY | 41 | −34.043 | −34.843 |
| 178 | THR | 37 | −37.496 | −37.439 |
| 179 | TYR | 35 | −29.272 | −29.931 |
| 180 | ALA | 38 | −22.821 | −24.566 |
| 181 | HIS | 27 | −19.131 | −20.036 |
| 182 | THR | 36 | 13.883 | −16.427 |
| 183 | VAL | 21 | −10.677 | −11.864 |
| 184 | ASN | 24 | −7.868 | −9.723 |
| 185 | ARG | 25 | −10.561 | −12.221 |
| 186 | ASN | 10 | −5.283 | −5.825 |
| 187 | TRP | 32 | −5.555 | −8.596 |
| 188 | TYR | 23 | −5.828 | −7.803 |
| 189 | SER | 26 | −2.543 | −5.24 |
| 190 | ASP | 20 | −1.535 | −3.64 |
| 191 | ALA | 23 | 1.261 | −1.529 |
| 192 | ASP | 28 | −0.012 | −2.886 |
| 193 | MET | 28 | −1.918 | −4.918 |
| 194 | PRO | 12 | −1.339 | −2.565 |
| 195 | ALA | 0 | −0.704 | −0.623 |
| 196 | SER | 8 | −2.563 | −3.186 |
| 197 | ALA | 27 | −4.929 | −7.467 |
| 198 | ARG | 16 | −2.99 | −4.486 |
| 199 | GUN | 3 | −1.446 | −1.625 |
| 200 | GLU | 18 | −4.144 | −5.738 |
| 201 | GLY | 6 | −4.243 | −4.445 |
| 202 | CYS | 37 | −7.573 | −10.957 |
| 203 | GLN | 21 | −8.104 | −9.587 |
| 204 | ASP | 30 | −8.648 | −11.104 |
| 205 | ILE | 36 | −12.918 | 15.572 |
| 206 | ALA | 37 | −10.56 | −13.6 |
| 207 | THR | 22 | −9.511 | −10.947 |
| 208 | GLN | 38 | −15.327 | −17.935 |
| 209 | LEU | 45 | −19.725 | −22.632 |
| 210 | ILE | 30 | −15.416 | −17.093 |
| 211 | SER | 16 | −12.65 | −13.036 |
| 212 | ASN | 33 | −17.93 | −19.663 |
| 213 | MET | 31 | −22.301 | −23.301 |
| 214 | ASP | 19 | −21.657 | −21.352 |
| 215 | ILE | 39 | −28.253 | −29.489 |
| 216 | ASP | 34 | −25.894 | −26.826 |
| 217 | VAL | 40 | −26.513 | −28.064 |
| 218 | ILE | 42 | −24.212 | −26.258 |
| 219 | LEU | 39 | −21.653 | 23.648 |
| 220 | GLY | 34 | −17.9 | −19.752 |
| 221 | GLY | 26 | −10.913 | −12.648 |
| 222 | GLY | 37 | −6.609 | −10.104 |
| 223 | ARG | 26 | −2.499 | −5.202 |
| 224 | LYS | 23 | 1.569 | −1.286 |
| 225 | TYR | 33 | −2.685 | −6.171 |
| 226 | MET | 32 | −4.278 | −7.466 |
| 227 | PHE | 25 | 0.253 | −2.651 |
| 228 | PRO | 7 | 1.931 | 0.904 |
| 229 | MET | 8 | 3.843 | 2.481 |
| 230 | GLY | 3 | 4.826 | 3.926 |
| 233 | THR | 22 | 2.818 | −0.036 |
| 232 | PRO | 5 | 3.353 | 2.392 |
| 233 | AS& | 25 | 3.292 | 0.038 |
| 234 | PRO | 19 | 1.042 | −1.262 |
| 235 | GLU | 28 | 2.964 | −0.597 |
| 236 | TYR | 20 | 5.779 | 2.835 |
| 237 | PRO | 3 | 5.426 | 4.457 |
| 238 | ALA | 0 | 5.367 | 4.75 |
| 239 | ASP | 13 | 5.81 | 3.877 |
| 240 | ALA | 2 | 5.633 | 4.755 |
| 243 | SER | 1 | 6.875 | 5.97 |
| 242 | GLN | 18 | 6.646 | 3.812 |
| 243 | ASN | 12 | 4.834 | 2.898 |
| 244 | GLY | 20 | 1.681 | −0.812 |
| 245 | ILE | 23 | 2.491 | −0.44 |
| 246 | ARG | 26 | 1.136 | −1.985 |
| 247 | LEU | 12 | 1.763 | 0.181 |
| 248 | ASP | 25 | 1.868 | −1.222 |
| 249 | GLY | 0 | 1.76 | 1.557 |
| 250 | LYS | 24 | 0.401 | −2.405 |
| 251 | ASN | 9 | −1.504 | −2.36 |
| 252 | LEU | 35 | −4.204 | −7.745 |
| 253 | VAL | 28 | −7.605 | −9.951 |
| 254 | GLN | 9 | −5.364 | −5.782 |
| 255 | GLU | 19 | −4.652 | −6.302 |
| 256 | TRP | 33 | −8.689 | −11.485 |
| 257 | LEU | 15 | −7.662 | −8.506 |
| 258 | ALA | 7 | −5.85 | −5.982 |
| 259 | LYS | 21 | −7.931 | −9.434 |
| 260 | HIS | 23 | −8.847 | 10.474 |
| 261 | GLN | 0 | −7.972 | −7.055 |
| 262 | GLY | 15 | −10.006 | −10.58 |
| 263 | ALA | 19 | −11.796 | 12.625 |
| 264 | TRP | 21 | −11.124 | −12.26 |
| 265 | TYR | 20 | −9.562 | −10.762 |
| 266 | VAL | 32 | −9.35 | −11.955 |
| 267 | TRP | 21 | −7.262 | −8.842 |
| 268* | ASN | 10 | −6.497 | −6.9 |
| 269 | ARG | 15 | −8.453 | −9.206 |
| 270 | THR | 4 | −6.376 | −6.103 |
| 271 | GLU | 18 | −9.107 | −10.13 |
| 272 | LEU | 29 | −12.563 | −14.453 |
| 273 | MET | 11 | −11.751 | −11.665 |
| 274 | GLN | 13 | −11.025 | −11.252 |
| 275 | ALA | 30 | −12.751 | −14.735 |
| 276 | SER | 25 | −15.19 | −16.319 |
| 277 | LEU | 7 | −13.695 | −12.925 |
| 278 | ASP | 19 | −11.055 | −11.969 |
| 279 | GLN | 0 | −8.533 | −7.551 |
| 280 | SER | 4 | −8.836 | −8.279 |
| 281 | VAL | 26 | −13.644 | −15.065 |
| 282 | THR | 13 | −13.513 | −13.454 |
| 283 | HIS | 19 | −16.546 | −16.828 |
| 284 | LEU | 41 | −17.256 | −19.987 |
| 285 | MET | 31 | −14.916 | −16.766 |
| 286 | GLY | 37 | −13.815 | −16.482 |
| 287 | LEU | 32 | −10.419 | −12.901 |
| 288 | PHE | 36 | −8.983 | −12.09 |
| 289 | GLU | 26 | −3.761 | −6.319 |
| 290 | PRO | 19 | 1.388 | −0.956 |
| 293 | GLY | 13 | −0.486 | −1.695 |
| 292 | ASP | 22 | −5.449 | −7.353 |
| 293 | THR | 43 | −5.665 | −9.959 |
| 284 | LYS | 19 | −1.629 | −3.627 |
| 295 | TYR | 21 | −4.013 | −5.966 |
| 296 | GLU | 29 | −3.898 | −6.785 |
| 297 | ILE | 21 | −0.863 | −3.179 |
| 298 | HIS | 19 | 3.537 | 0.946 |
| 293 | ARG | 26 | 0.642 | −2.422 |
| 300 | ASP | 13 | 1.621 | −0.06 |
| 301 | PRO | 4 | −1.667 | −1.935 |
| 302 | THR | 1 | 0.284 | 0.136 |
| 303 | LEU | 17 | −1.181 | −3 |
| 304 | ASP | 27 | −7.2 | −9.477 |
| 305 | PRO | 36 | −11.766 | −14.553 |
| 306 | SER | 19 | −14.99 | −15.451 |
| 307 | LEU | 37 | −23.95 | −25.451 |
| 308 | MET | 19 | −21.854 | −21.525 |
| 309 | GLU | 24 | −18.668 | −19.282 |
| 310 | MET | 44 | −23.682 | −26.018 |
| 311 | THR | 31 | −29.94 | −30.062 |
| 312 | GLU | 18 | −22.702 | −22.161 |
| 313 | ALA | 35 | −20.112 | −21.824 |
| 314 | ALA | 41 | −28.574 | −30.003 |
| 315 | LEU | 26 | −30.164 | −29.685 |
| 316 | ARG | 20 | −21.442 | −21.276 |
| 317 | LEU | 35 | −23.393 | −24.728 |
| 318 | LEU | 37 | −28.166 | −29.182 |
| 319 | SER | 21 | −25.089 | −24.619 |
| 320 | ARG | 20 | −16.939 | −17.291 |
| 321 | ASN | 29 | −19.46 | −20.557 |
| 322 | PRO | 3 | −16.019 | −14.521 |
| 323 | ARG | 14 | −20.252 | −19.533 |
| 324 | GLY | 34 | −28.321 | −28.974 |
| 325 | PHE | 33 | −35.871 | −35.541 |
| 326 | TYR | 36 | −43.103 | −42.287 |
| 327 | LEU | 43 | −44.606 | −44.422 |
| 328 | PHE | 43 | −43.503 | −43.445 |
| 329 | VAL | 36 | −40.736 | −40.191 |
| 330 | GLU | 39 | −36.279 | −36.592 |
| 331 | GLY | 42 | −29.526 | −30.96 |
| 332 | GLY | 35 | 25.633 | −26.71 |
| 333 | ARG | 30 | −14.36 | −16.159 |
| 334 | ILE | 43 | −17.264 | −20.224 |
| 335 | ASP | 33 | −16.683 | −18.559 |
| 336 | HIS | 25 | −7.554 | −9.56 |
| 337 | GLY | 38 | −9.078 | −12.404 |
| 338 | HIS | 35 | −9.33 | −12.282 |
| 339 | HIS | 13 | −3.834 | −4.888 |
| 340 | GLU | 20 | −2.832 | −4.806 |
| 341 | GLY | 23 | −4.987 | −7.059 |
| 342 | VAL | 18 | −4.973 | −6.471 |
| 343 | ALA | 39 | −9.309 | 12.723 |
| 344 | TYR | 19 | −6.808 | −8.21 |
| 345 | GLN | 28 | −6.403 | −8.887 |
| 346 | ALA | 42 | −13.416 | −16.703 |
| 347 | LEU | 40 | −14.776 | −17.677 |
| 348 | THR | 22 | −10.149 | −11.512 |
| 349 | GLU | 36 | −15.275 | −17.658 |
| 350 | ALA | 40 | −21.474 | −23.604 |
| 351 | VAL | 23 | −17.662 | −18.276 |
| 352 | MET | 34 | −18.411 | −20.204 |
| 353 | PHE | 43 | −27.739 | −29.494 |
| 354 | ASP | 33 | −26.135 | −26.925 |
| 355 | ASP | 21 | −21.102 | −21.09 |
| 356 | ALA | 41 | −26.748 | −28.387 |
| 357 | ILE | 36 | −33.366 | −33.669 |
| 358 | GLU | 15 | −24.487 | −23.396 |
| 359 | ARG | 23 | −20.55 | −20.831 |
| 360 | ALA | 47 | −30.792 | −32.656 |
| 361 | GLY | 21 | −28.209 | −27.38 |
| 362 | GLN | 14 | −18.779 | −18.229 |
| 363 | LEU | 26 | −20.432 | −21.072 |
| 364 | THR | 41 | −27.395 | −28.96 |
| 365 | SER | 16 | −23.753 | −22.861 |
| 366 | GLU | 23 | −27.655 | −27.12 |
| 367 | GLU | 8 | 22.282 | −20.639 |
| 368 | ASP | 19 | −25.576 | −24.82 |
| 369 | THR | 31 | −34.2 | −33.832 |
| 370 | LEU | 37 | −39.566 | −39.271 |
| 371 | THR | 29 | −42.934 | −41.331 |
| 372 | LEU | 40 | −45.02 | −44.443 |
| 373 | VAL | 33 | −42.181 | 41.125 |
| 374 | THR | 43 | −42.039 | 42.15 |
| 375 | ALA | 37 | −35.432 | −35.613 |
| 376 | ASP | 47 | −34.536 | −35.97 |
| 377 | HIS | 32 | −27.491 | −28.01 |
| 378 | SER | 28 | −20.738 | −21.573 |
| 379 | HIS | 42 | −15.315 | −18.383 |
| 380 | VAL | 21 | −11.58 | −12.663 |
| 381 | PHE | 36 | −13.054 | −15.692 |
| 382 | SER | 17 | −12.514 | −13.03 |
| 383 | PHE | 31 | −13.274 | −15.313 |
| 384 | GLY | 13 | 11.139 | −11.353 |
| 385 | GLY | 30 | −9.517 | −11.873 |
| 386 | TYR | 4 | −8.465 | −7.951 |
| 387 | THR | 28 | −10.179 | −12.229 |
| 388 | LEU | 13 | −8.64 | −9.142 |
| 389 | ARG | 12 | −9.485 | −9.774 |
| 390 | GLY | 13 | −9.461 | −9.868 |
| 391 | SER | 24 | −10.805 | −12.322 |
| 392 | SER | 20 | −10.294 | −11.41 |
| 393 | ILE | 33 | −10.774 | −13.33 |
| 394 | PHE | 26 | −8.821 | −10.797 |
| 395 | GLY | 23 | −10.175 | −11.65 |
| 396 | LEU | 19 | −9.142 | −10.276 |
| 397 | ALA | 21 | −9.492 | −10.815 |
| 398 | PRO | 11 | −7.754 | −8.127 |
| 399 | SER | 6 | −7.653 | −7.463 |
| 400 | LYS | 7 | −5.811 | −5.948 |
| 401 | ALA | 19 | −5.353 | 6.922 |
| 402 | GLN | 0 | −2.542 | −2.25 |
| 403 | ASP | 11 | −1.347 | −2.457 |
| 404 | SER | 0 | −3.271 | −2.895 |
| 405 | LYS | 16 | −3.807 | −5.209 |
| 406 | ALA | 18 | −6.752 | −8.046 |
| 407 | TYR | 22 | −8.754 | −10.277 |
| 408 | THR | 35 | −10.376 | −13.208 |
| 409 | SER | 29 | −11.052 | −13.116 |
| 410 | ILE | 40 | −10.767 | 14.128 |
| 411 | LEU | 23 | −9.091 | −10.69 |
| 412 | TYR | 36 | −7.951 | −11.177 |
| 413 | GLY | 13 | −5.781 | −6.611 |
| 414 | ASN | 13 | −3.671 | −4.744 |
| 415 | GLY | 29 | −3.115 | −6.092 |
| 416 | PRO | 14 | −1.899 | −3.291 |
| 417 | GLY | 36 | −3.898 | −7.59 |
| 418 | TYR | 16 | −3.525 | −4.96 |
| 419 | VAL | 19 | −2.356 | −4.27 |
| 420 | PHE | 10 | −2.558 | −3.414 |
| 421 | ASN | 8 | −0.701 | −1.541 |
| 422 | SER | 2 | 0.065 | −0.173 |
| 423 | GLY | 0 | −0.171 | −0.152 |
| 424 | VAL | 10 | −1.049 | ·2.078 |
| 425 | ARG | 18 | −2.583 | −4.356 |
| 426 | PRO | 18 | −1.857 | −3.714 |
| 427 | ASP | 9 | −3.733 | −4.338 |
| 428 | VAL | 31 | −6.155 | −9.012 |
| 429* | ASN | 2 | −6.559 | −6.035 |
| 430 | GLU | 13 | −6.639 | −7.371 |
| 431 | SER | 0 | 5.72 | −5.063 |
| 432 | GLU | 16 | −5.663 | −6.851 |
| 433 | SER | 26 | −6.565 | −8.8 |
| 434 | GLY | 14 | −6.358 | −7.237 |
| 435 | SER | 13 | −3.762 | −4.825 |
| 436 | PRO | 8 | −2.25 | −2.931 |
| 437 | ASP | 1 | −1.326 | −1.289 |
| 438 | TYR | 28 | −4.01 | −6.769 |
| 439 | GLN | 23 | −3.93 | −6.123 |
| 440 | GLN | 36 | −6.443 | −9.842 |
| 441 | GLN | 24 | −6.793 | −8.772 |
| 442 | ALA | 27 | −5.906 | −8.332 |
| 443 | ALA | 9 | −4.446 | −4.97 |
| 444 | VAL | 13 | −2.742 | −3.691 |
| 445 | PRO | 5 | −0.878 | −1.352 |
| 446 | 1.80 | 18 | 0.07 | −2.008 |
| 447 | SER | 0 | 2.461 | 2.178 |
| 448 | SER | 11 | 0.536 | −0.791 |
| 449 | GLU | 37 | −3.597 | −7.438 |
| 450 | THR | 14 | −4.355 | −5.464 |
| 451 | HIS | 30 | −12.311 | −14.345 |
| 452 | GLY | 29 | −14.271 | −15.965 |
| 453 | GLY | 12 | −13.51 | −13.337 |
| 454 | GLU | 13 | −16.801 | −16.364 |
| 455 | ASP | 15 | −20.272 | −19.666 |
| 456 | VAL | 42 | −29.13 | −30.61 |
| 457 | ALA | 22 | −33.365 | −32.058 |
| 458 | VAL | 36 | −37.806 | −37.598 |
| 459 | PHE | 25 | −38.318 | −36.787 |
| 460 | ALA | 36 | −38.206 | −37.952 |
| 461 | ARG | 19 | −34.002 | −32.277 |
| 462 | GLY | 38 | −32.215 | −32.88 |
| 463 | PRO | 32 | −30.4 | −30.584 |
| 464 | GLN | 28 | −31.026 | −30.678 |
| 465 | ALA | 33 | −34.346 | −34.192 |
| 466 | HIS | 14 | −28.904 | −27.19 |
| 467 | LEU | 26 | −30.299 | −29.805 |
| 468 | VAL | 37 | −32.134 | −32.693 |
| 469 | HIS | 8 | 25.387 | −23.388 |
| 470 | GLY | 7 | −19.811 | −18.338 |
| 471 | VAL | 5 | −19.255 | −17.616 |
| 472 | GLN | 32 | −26.119 | −26.795 |
| 473 | GLU | 17 | −27.045 | −25.889 |
| 474 | GLN | 42 | −32.939 | −33.981 |
| 475 | SER | 19 | −34.071 | −32.338 |
| 476 | PHE | 27 | −35.021 | −34.099 |
| 477 | VAL | 44 | −42.085 | −42.305 |
| 478 | ALA | 33 | −42.276 | −41.209 |
| 479 | HIS | 14 | −36.778 | −34.159 |
| 480 | VAL | 36 | −39.7 | −39.274 |
| 481 | MET | 42 | −42.715 | −42.633 |
| 482 | ALA | 27 | −39.404 | −37.978 |
| 483 | PHE | 20 | −34.879 | −33.168 |
| 484 | ALA | 37 | −35.119 | −35.335 |
| 485 | ALA | 38 | −33.591 | −34.098 |
| 486 | CYS | 17 | −27.643 | −26.419 |
| 487 | LEU | 36 | −30.441 | −31.08 |
| 488 | GLU | 13 | −25.646 | −24.192 |
| 489 | PRO | 10 | −22.46 | −21.028 |
| 490 | TYR | 25 | −24.248 | −24.334 |
| 491 | THR | 7 | −20.616 | −19.051 |
| 492 | ALA | 0 | −17.345 | −15.35 |
| 493 | CYS | 27 | −21.021 | −21.709 |
| 494 | ASP | 0 | −15.571 | −13.78 |
| 495 | LEU | 31 | −18.257 | −19.722 |
| 496 | ALA | 9 | −13.383 | −12.879 |
| 497 | PRO | 5 | −15.939 | −14.681 |
| 498 | PRO | 26 | −18.958 | −19.768 |
| 499 | ALA | 15 | −15.131 | −15.116 |
| 500 | CYS | 4 | −12.778 | −11.768 |
| 501 | THR | 19 | −11.758 | −12.591 |
| 502 | THR | 4 | −5.513 | −5.339 |
| 503 | ASP | 7 | −5.966 | −6.085 |
| 504 | ALA | 0 | −4.636 | −4.102 |
| 505 | ALA | 5 | −4.478 | −4.538 |
| 506 | HIS | 2 | −4.755 | −4.438 |
| 507 | PRO | 3 | −5.501 | −5.214 |
| 508 | VAL | 0 | −5.828 | −5.158 |
| 509 | ALA | 2 | −6.295 | −5.801 |
| 510 | ALA | 0 | −6.584 | −5.827 |
| 511 | SER | 5 | −6.737 | −6.537 |
| 512 | LEU | 1 | −6.617 | −5.971 |
| 513 | PRO | 3 | −6.8 | −6.363 |
| 514 | LEU | 0 | −7.762 | −6.87 |
| 515 | LEU | 2 | −8.318 | −7.592 |
| 516 | ALA | 0 | −8.769 | −7.761 |
| 517 | GLY | 3 | −8.365 | −7.748 |
| 518 | THR | 0 | −8.732 | −7.728 |
| 519 | LEU | 0 | −8.652 | −7.657 |
| 520 | LEU | 0 | −9.446 | −8.36 |
| 521 | LEU | 1 | −9.414 | −8.447 |
| 522 | LEU | 0 | −8.398 | −7.432 |
| 523 | GLY | 3 | −7.579 | −7.053 |
| 524 | ALA | 0 | −7.106 | −6.289 |
| 525 | SER | 0 | −6.318 | −5.592 |
| 526 | ALA | 0 | −5.203 | −4.605 |
| 527 | ALA | 1 | −3.97 | −3.629 |
| S28 | PRO | 0 | −2.601 | −2.302 |

### Example 3

A recombinant monoclonal antibody with the following properties was generated using a peptide synthesized from the human sequence of intestinal alkaline phosphatase.

### Peptide Antigen:

Human intestinal alkaline phosphatase epitope
(6-hsALPI; also named peptide 6):
PGYVENSGVRPDVNESESGSPDY
(SEQ ID NO: 30; human intestinal alkaline
phosphatase protein aa416-aa438)
Calculated molecular weight of 6-hsALPI = 2472.53 g/mol.
CDI022E3 heavy chain amino acid sequence:
(SEQ ID NO: 20)
METGLRWLLLVAVLKGVQCQSLEESGGRLVTPGTPLTLTCTVSGIDLSTYAMGWVRQAPG
EGLEWIGTIGVSGSTYYASWAKGRFTISKTSTTVDLKMTSPATEDTATYFCARGSVWGPG
TLVTVSLGQPKAPSVFPLAPCCGDTPSSTVTLGCLVKGYLPEPVTVTWNSGTLINGVRTE
PSVROSSGLYSLSSVVSVTSSSQPVTCNVAHPATNTKVDKTVAPSTCSKPMCPPPELPGG
PSVFIFPPKPKDTLMISRTPEVTCVVVDVSQDDPEVOFTWYINNEQVRTARPPLREQQEN
STIRVVSTLPIAHQDWLRGKEFKCKVHNKALPAPIEKTISKARGQPLEPKVYTMGPPREE
LSSRSVSLTCMINGFYPSDISVEWEKNGKAEDNYKTTPTVLDSDGSYFLYSKLSVPTSEW
QRGDVFTCSVMHEALHNHYTQKSISRSPGK*
CDI022E3 light chain amino acid sequence:
(SEQ ID NO: 21)
MDTRAPTQLLGLLLLWLPGATFAQVLTQTPASVEAAVGGTVTIKCQASQSISTWLAWYQQ
KPGQAPKRLIYSASTLASGVSSRFKGSGSGTEFTLTISDLECADAATYYCRCSYGKSYGE
AFGGGTEVVVKGDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQSFNRGDC*
Heavy chain CDR1 - CDI022E3
(SEQ ID NO: 22)
TVSGIDLSTYAMG
Heavy chain CDR2 - CDI022E3
(SEQ ID NO: 23 or SEQ ID NO: 31)
(SEQ ID NO: 23)
TIGVSGSTY;
or
(SEQ ID NO: 31)
TIGVSGSTYYASWAKG
Heavy chain CDR3 - CDI022E3
(SEQ ID NO: 24)
ARGSV
Light chain CDR1 - CDI022E3
(SEQ ID NO: 25)
QASQSISTWLA
Light chain CDR2 - CDI022E3
(SEQ ID NO: 26)
YSASTLAS
Light chain CDR3 - CDI022E3
(SEQ ID NO: 27)
RCSYGKSYGEA
CDI022E3_H (heavy chain DNA sequence):
(SEQ ID NO: 28)
ATGGAGACTGGGCTGCGCTGGCTTCTCCTGGTCGCTGTGCTCAAAGGTGTCCAGTGTCAG
TCGCTGGAGGAGTCCGGGGGTCGCCTGGTCACGCCTGGGACACCCCTGACACTCACCTGC
ACAGTCTCTGGAATCGACCTCAGTACCTATGCAATGGGCTGGGTCCGCCAGGCTCCAGGG
GAGGGGCTGGAATGGATCGGGACCATTGGAGTTAGTGGTAGCACATACTACGCGAGCTGG
GCGAAAGGCCGATTCACCATCTCCAAAACCTCGACCACGGTGGATCTCAAAATGACCAGT
CCGGCAACCGAGGACACGGCCACCTATTTCTGTGCCAGAGGAAGCGTCTGGGGCCCAGGC
ACCCTGGTCACCGTCTCCTTGGGGCAACCTAAGGCTCCATCAGTCTTCCCACTGGCCCCC
TGCTGCGGGGACACACCCAGCTCCACGGTGACCCTGGGCTGCCTGGTCAAAGGCTACCTC
CCGGAGCCAGTGACCGTGACCTGGAACTCGGGCACCCTCACCAATGGGGTACGCACCTTC
CCGTCCGTCCGGCAGTCCTCAGGCCTCTACTCGCTGAGCAGCGTGGTGAGCGTGACCTCA
AGCAGCCAGCCCGTCACCTGCAACGTGGCCCACCCAGCCACCAACACCAAAGTGGACAAG
ACCGTTGCGCCCTCGACATGCAGCAAGCCCATGTGCCCACCCCCTGAACTCCCGGGGGGA
CCGTCTGTCTTCATCTTCCCCCCAAAACCCAAGGACACCCTCATGATCTCACGCACCCCC
GAGGTCACATGCGTGGTGGTGGACGTGAGCCAGGATGACCCCGAGGTGCAGTTCACATGG
TACATAAACAACGAGCAGGTGCGCACCGCCCGGCCGCCGCTACGGGAGCAGCAGTTCAAC
AGCACGATCCGCGTGGTCAGCACCCTCCCCATCGCGCACCAGGACTGGCTGAGGGGCAAG
GAGTTCAAGTGCAAAGTCCACAACAAGGCACTCCCGGCCCCCATCGAGAAAACCATCTCC
AAAGCCAGAGGGCAGCCCCTGGAGCCGAAGGTCTACACCATGGGCCCTCCCCGGGAGGAG
CTGAGCAGCAGGTCGGTCAGCCTGACCTGCATGATCAACGGCTTCTACCCTTCCGACATC
TCGGTGGAGTGGGAGAAGAACGGGAAGGCAGAGGACAACTACAAGACCACGCCGACCGTG
CTGGACAGCGACGGCTCCTACTTCCTCTACAGCAAGCTCTCAGTGCCCACGAGTGAGTGG
CAGCGGGGCGACGTCTTCACCTGCTCCGTGATGCACGAGGCCTTGCACAACCACTACACG
CAGAAGTCCATCTCCCGCTCTCCGGGTAAATAG
CDI022E3_L (light chain DNA sequence):
(SEQ ID NO: 29)
ATGGACACGAGGGCCCCCACTCAGCTGCTGGGGCTCCTGCTGCTCTGGCTCCCAGGTGCC
ACATTTGCCCAAGTGCTGACCCAGACTCCAGCCTCCGTGGAGGCAGCTGTGGGAGGCACA
GTCACCATCAAGTGCCAGGCCAGTCAGAGCATTAGCACCTGGTTAGCCTGGTATCAGCAG
AAACCAGGGCAGGCTCCCAAGCGCCTGATCTATTCTGCATCCACTCTGGCATCTGGGGTC
TCATCGCGGTTCAAAGGCAGTGGATCTGGGACAGAGTTCACTCTCACCATCAGCGACCTG
GAGTGTGCCGATGCTGCCACTTACTACTGTCGATGTAGTTATGGTAAAAGTTATGGTGAG
GCTTTCGGCGGAGGGACCGAGGTGGTGGTCAAAGGTGATCCAGTTGCACCTACTGTCCTC
ATCTTCCCACCAGCTGCTGATCAGGTGGCAACTGGAACAGTCACCATCGTGTGTGTGGCG
AATAAATACTTTCCCGATGTCACCGTCACCTGGGAGGTGGATGGCACCACCCAAACAACT
GGCATCGAGAACAGTAAAACACCGCAGAATTCTGCAGATTGTACCTACAACCTCAGCAGC
ACTCTGACACTGACCAGCACACAGTACAACAGCCACAAAGAGTACACCTGCAAGGTGACC
CAGGGCACGACCTCAGTCGTCCAGAGCTTCAATAGGGGTGACTGTTAG

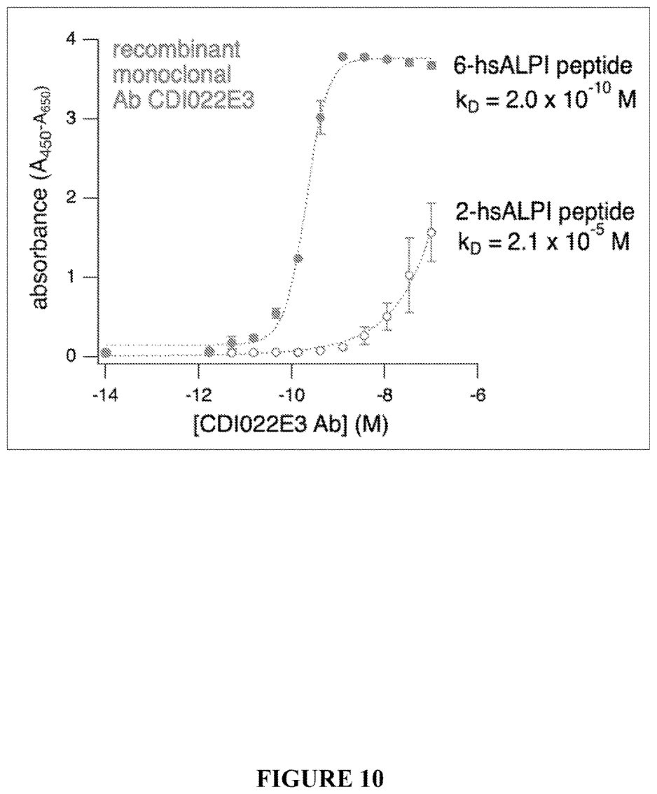

Affinity and specificity determination for CDI022E3 [See FIG. 10]: K_{D, CDI022E3 Ab}=200 μM, when using 2 ug/mL (0.8 μM) of peptide antigen 6-hsALPI. K_{D, CDI022E3 Ab}=21 μM, when using 2 μg/mL (1.1 μM) of peptide antigen 2-hsALPI.

### Example 4

### Glycopeptide Analysis

Antibodies are well-known to be glycosylated, and this post-translational modification is critical for their antibody effector properties. In general, antibody glycosylation defines structure of the antibody Fc region and determines which Fc receptors it can bind to in order to recruit effector cells. Antibody glycosylation can be harnessed to improve the efficacy of monoclonal therapeutics. Antibody glycosylation can be modulated by vaccination, indicated that rational immunogen design can seek to elicit a specific antibody glycosylation response.

Glycosylation is a common post-translational modified for IgG antibodies produced by mammalian cells, i.e. Chinese hamster ovary (CHO) cells, which are frequently used for production. IgG1 molecules contain a single N-linked glycan at Asn297 in the CH2 domain in each of the two heavy chain Fc region. During synthesis of Nglycans, multiple sugar moieties can be added to form different glycoforms, e.g., G0, G1, G2, afucosylated complex. Glycoforms can be necessary to achieve therapeutic efficacy. The observed high heterogeneity of glycoforms can impact antibody stability, pharmacokinetics (PK), efficacy, and immunogenicity of mAbs. In addition, glycoform diversity plays an important role for complement-dependent cytotoxicity (CDC) and antibody-dependent cell-mediated cytotoxicity (ADCC) functions through modulating the binding to the Fcγ receptor. Targeting of specific glycoforms can be pursued via molecular glycosylation engineering, but can also be affected by cell culture conditions.

Terminal sugar residues, such as mannose, sialic acids, fucose, or galactose, influence therapeutic mAbs positively or negatively. Mannosylation, which has significant undesirable effects on the PK of glycoproteins, can cause a decreased half life of mAbs. Moreover, terminal galactose residues can enhance CDC activities and Fc-Clq interactions, whereas core fucose can decrease ADCC and Fc-FcγR binding.

### Glycopeptide Analysis of Monoclonal Antibody Sample

### Materials and Methods:

Protein digestion was performed. Briefly, provided proteins were treated with dithiothreitol (DTT) and iodoacetamide (IAA) to reduce and alkylate prior to overnight trypsin digestion. LC-MS/MS analysis was performed using a Thermo Eclipse mass spectrometer coupled to an Ultimate 3000 RSLCnano system. Data was analyzed using Byonic ver5 software and manual interpretation, including looking for diagnostic ions of NeuAc₂ (Medzihradszky et al. (2015) Anal. Chem). Delta mod score was >10 for regular glycopeptides, for acetylation searches delta mod score of >1 was used.

### Results:

Nano LC-MS/MS analyses confirmed the presence of monoclonal antibody protein in samples provided. Overall quality of recovered peptides was high and recovery of peptide analyses was repeatable on independent antibody samples. Byonic search found 1 glycosylation site in the heavy chain and 1 glycosylation site on the light chain.

TABLE 6
Percent coverage and present glycosylation
sites of provided samples:

| | Protein | N-gly | | Protein | N-gly |
|---|---|---|---|---|---|
| | coverage | residue | | coverage | residue |
| Sample 1 | (%) | site | Sample 2 | (%) | site |
| Heavy chain | 90.7 | 304 | Heavy chain | 87 | 304 |
| Light chain | 91.2 | 202 | Light chain | 91.2 | 202 |

Reagents used in analyses were purchased from Sigma Aldrich. Data acquisition was performed on a Thermo Scientific Orbitrap Eclipse Tribrid mass spectrometer tandem NanoFlow-LC system. Data analysis was performed by using FreeStyle 1.8, Xcalibur 3.0, and Byonic v4.0.12 software. Peptide analysis and site mapping were performed using LC-MS/MS. Two provided samples were reduced with DTT, alkylated with iodoacetamide, and the protein was digested with 0.5 μg/μl sequencing-grade trypsin and chymotrypsin (later) at 37° C. for 16 h. Peptides were analyzed on an Ultimate 3000 RSLCnano connected to a Thermo Eclipse mass spectrometer. Nano-LC columns of 15 cm length with 75 μm internal diameter, filled with 3 μm C18 reverse phase material were used for chromatographic separation of the samples. The separation conditions were low to high acetonitrile in a solution containing 0.1% formic acid, and the separation time was 60 minutes. The precursor ion scan was acquired at 120,000 resolution in the Orbitrap analyzer and precursors at a time frame of 3 s were selected for subsequent fragmentation using HCD. Charge state screening was enabled, and precursors with unknown charge state or a charge state of +1 were excluded. Dynamic exclusion was enabled (exclusion duration of 30 s). The fragment ions were analyzed on an Orbitrap for HCD at 30,000 resolution The resulting glycoproteomic data were processed with Byonic (v4.0.12), and searched against the provided sequences and a catalogue of more than 70+ mammalian N-glycans and 9 common O-glycans. The precursor mass tolerance and fragment mass tolerances were set to 5 ppm and 10 ppm respectively. Additional modifications including deamidation of N and Q, carboxymethylation of C, and oxidation of precursor were also included in the search. Assignments were made using Byonic software and manual interpretation.

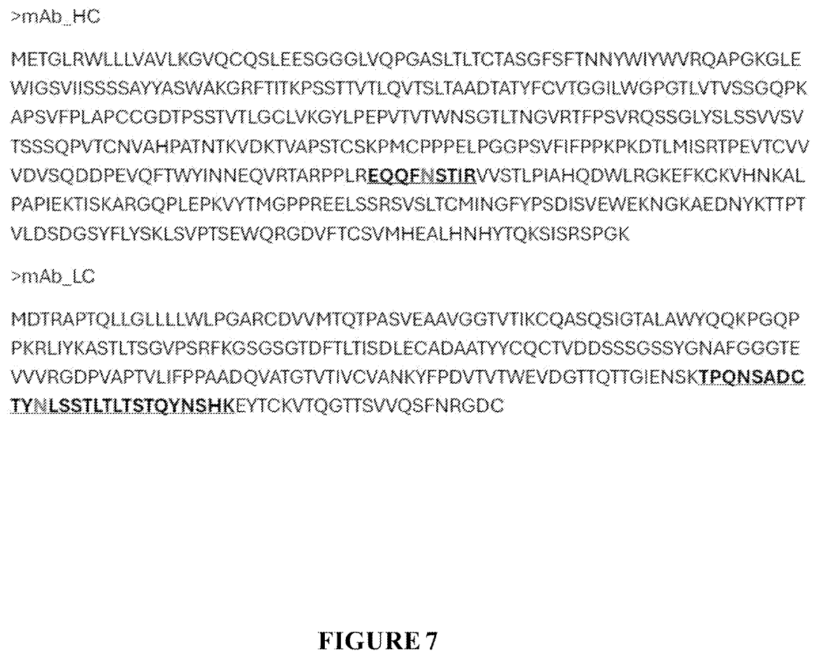

Referring to FIG. 7, a glycopeptide coverage map for rmAb CDI005E8 is shown. Amino Acids (AA) in bold and underlined are fragments that were chosen for RA calculations. AA in red are N-glycosylation sites.

rmAb CDI005E8_HC
METGLRWLLLVAVLKGVQCQSLEESGGGLVQPGASLTLTCTASGFSFTNNYWIYWV
RQAPGKGLEWIGSVIISSSSAYYASWAKGRFTITKPSSTTVTLQVTSLTAADTATYFC
VTGGILWGPGTLVTVSSGQPKAPSVFPLAPCCGDTPSSTVTLGCLVKGYLPEPVTVT
WNSGTLTNGVRTFPSVRQSSGLYSLSSVVSVTSSSQPVTCNVAHPATNTKVDKTVAP
STCSKPMCPPPELPGGPSVFIFPPKPKDTLMISRTPEVTCVVVDVSQDDPEVQFTWYIN
NEQVRTARPPLREQQF [symbol] STIRVVSTLPIAHQDWLRGKEFKCKVHNKALPAPIEKTIS
KARGQPLEPKVYTMGPPREELSSRSVSLTCMINGFYPSDISVEWEKNGKAEDNYKTT
PTVLDSDGSYFLYSKLSVPTSEWQRGDVFTCSVMHEALHNHYTQKSISRSPGK
rmAb CDI005E8_LC
MDTRAPTQLLGLLLLWLPGARCDVVMTQTPASVEAAVGGTVTIKCQASQSIGTALA
WYQQKPGQPPKRLIYKASTLTSGVPSRFKGSGSGTDFTLTISDLECADAATYYCQCT
VDDSSSGSSYGNAFGGGTEVVVRGDPVAPTVLIFPPAADQVATGTVTIVCVANKYFP
DVTVTWEVDGTTQTTGIENSKTPQNSADCTY [symbol] LSSTLTLTSTQYNSHKEYTCKVT
QGTTSVVQSFNRGDC

TABLE 7
Relative abundancies (RA) of identified glycoforms found on
EQQFNSTIR, Pos 304 (rmAb CDI005E8 HC):

| | | | Delta | | | | | | |
|---|---|---|---|---|---|---|---|---|---|
| | | | Mod. | | Obs. | Calc. | ppm | | Scan |
| Sequence | Glycans | RA | Score | z | m/z | m/z | er | Calc. | Time |
| EQQFN[+1241.45]STIR | HexNAc(3) | 3.5 | 507 | 3 | 788.6748 | 768.67 | 0.61 | 2364.01 | 17.9 |
| | Hex(3) | | | | | | | | |
| | Fuc(1) | | | | | | | | |
| EQQFN[+1444 53]STIR | HexNAc(4) | 60.1 | 513 | 2 | 1284.047 | 1284.05 | −0.15 | 2567.09 | 17.8 |
| | Hex(3) | | | | | | | | |
| | Fuc(1) | | | | | | | | |
| EQQFN[+1506.56]STIR | HexNAc(4) | 27.2 | 441 | 2 | 1365.074 | 1965.07 | −0.27 | 2729.14 | 17.6 |
| | Hex(4) | | | | | | | | |
| | Fuc(1) | | | | | | | | |
| EQQFN[+1768.63]STIR | HexNAc(4) | 2.9 | 342 | 2 | 1446.101 | 1446.10 | 0.39 | 2891.19 | 17.5 |
| | Hex(5) | | | | | | | | |
| | Fuc(1) | | | | | | | | |

TABLE 8
Relative abundancies (RA) of identified glycoforms found on
TPQNSADCTYNLSSTLTLTSTQYNSHK, Pos 202 (rmAb CDI005E8 LC):

| | | | Delta | | | | | | |
|---|---|---|---|---|---|---|---|---|---|
| | | RA | Mod. | | Obs. | Calc. | ppm | Clac. | Scan |
| Sequence | Glycans | % | Score | z | m/z | m/z | err. | MH | Time |
| TPQNSADCTYNLSSTLTLTS | NG | 89.9 | 1113 | 3 | 1011.468 | 1011.468 | −0.32 | 3032.3902 | 31.3 |
| TQYNSHK | | | | | | | | | |
| TPQNSADCTYN[+1540.52] | HexNAc(2) | 0.1 | 145 | 4 | 1143.985 | 1146.965 | 0.23 | 4572.9187 | 30.2 |
| LSSTLTLTSTQYNSHK | Hex(7) | | | | | | | | |
| TPQNSADCTYN[+1702.56] | HexNAc(2) | 5.2 | 512 | 4 | 1184.496 | 1184.498 | 0 | 4734.9717 | 30.0 |
| LSSTLTLTSTQYNSHK | Hex(8) | | | | | | | | |
| TPQNSADCTYN[+1664.63] | HexNAc(2) | 4.8 | 212 | 4 | 1225.009 | 1225.012 | −2.01 | 4697.0243 | 31.0 |
| LSSTLTLTSTQYNSHK | Hex(9) | | | | | | | | |

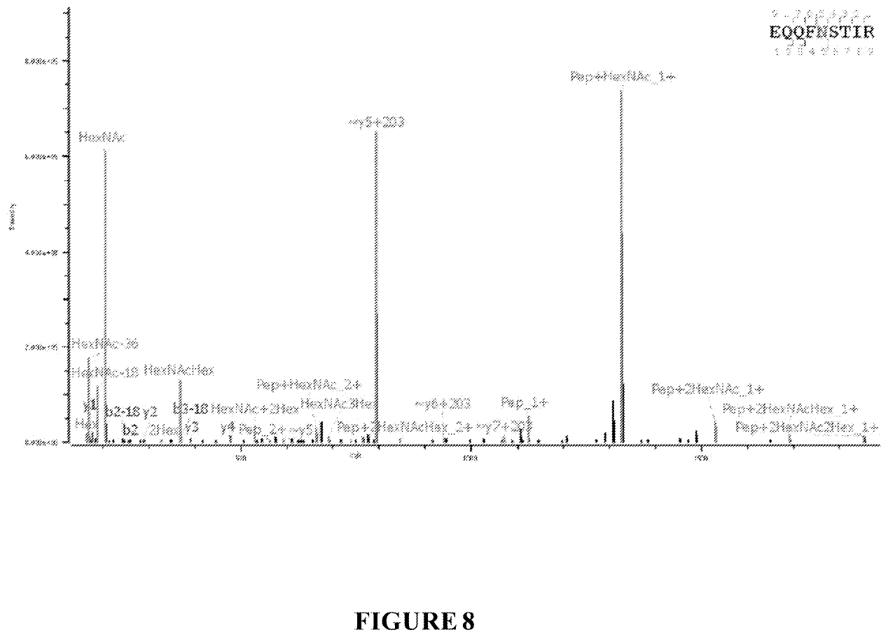

Referring to FIG. 8, a representative MS2 spectrum of glycopeptide found in rmAb CDI005E8 is shown. Peaks representing HexNAc (3) Hex (3) structure on the following peptide EQQFNSTIR (pos. 304) are shown. Glycoform structures are based on total mass and known biosynthetic pathways. The structures do not reflect sequencing or compositional analysis.

### Glycomic Analysis of Monoclonal Antibody Sample

### Materials and Methods:

### Processing of Glycoprotein and Release of N-Glycans:

100 μg of each sample was transferred to a clean Eppendorf tube and an equivalent volume of 25 mM DTT was added and incubated at 50° C. for 45 minutes to denature. The samples were then gradually desalted using 10 kDa cutoff spin filters by centrifuging at 14,000×g for 15 minutes. (Note: the 10 kDa cutoff spin filters were washed twice with 400 μL of 50 mM ammonium bicarbonate before use). The samples were then washed using 400 μL of 50 mM ammonium bicarbonate and centrifuged again at 14,000×g for 15 minutes. This step was repeated once more. The sample remaining in the filter was transferred to a clean Eppendorf tube and the N-glycans were released from proteins by adding PNGase F and incubating at 37° C. for 18 hours. The released N-glycans were separated from the O-glycoprotein using 10 kDa cutoff spin filter, purified using C18 cartridge, and were lyophilized before proceeding to permethylation.

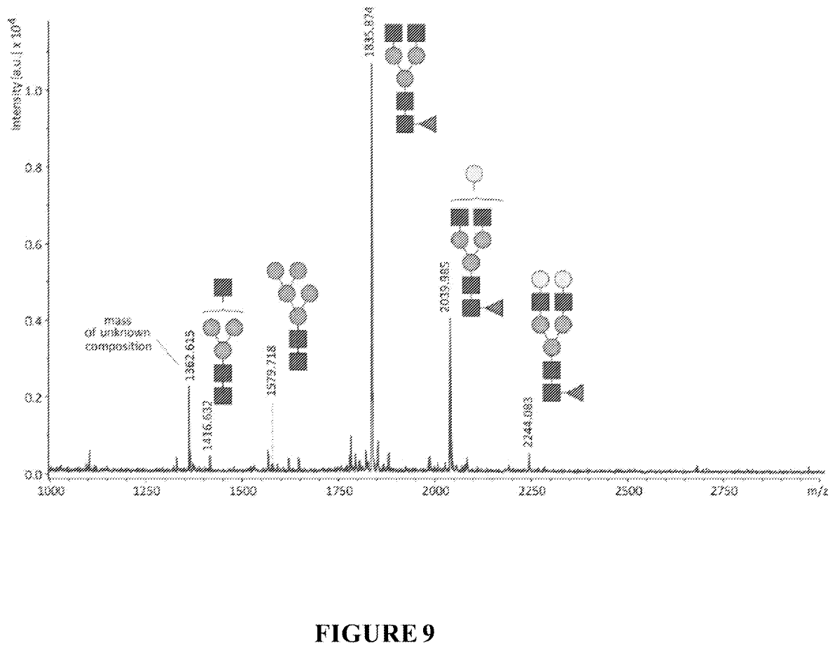

Referring to FIG. 9, MALDI spectrum of N-glycans identified in rmAb CDI005E8 is shown. For the major N-glycans, m/z is given together with a cartoon representation of the structure. Glycoforms are provided in Table 9.

TABLE 9
Relative abundancies of glycans identified in rmAb CDI005E8:

| Monoisotopic | | |
|---|---|---|
| Mass | | Percent |
| [M + Na⁺] | Glycan Text Composition | Composition |
| 1835.87 | HexNAc)₂(Deoxyhexose)₃ + (Man)₃(GlcNAc)₂ | 63.81 |
| 2039.99 | (Hex)₃(HexNAC)₂(Deoxyhexose)₃ + (Man)₃(GlcNAc)₂ | 24.49 |
| 1416.63 | (HexNAc)₃ + (Man)₃(GlcNAc)₂ | 2.92 |
| 2244.08 | (Hex)₂(HexNAc)₂(Deoxyhexose)₃ + (Man)₃(GlcNAc)₂ | 2.73 |
| 1620.74 | (Hex)₃(HexNAc)₃ + (Man)₃(GlcNAc)₂ | 2.42 |
| 1590.72 | (HexNAC)₃(Deoxyhexose)₃ + (Man)₃(GlcNAc)₂ | 1.37 |
| 1866,89 | (Hex)₃(HexNAc)₂ + (Man)₃(GlcNAc)₂ | 1.17 |
| 1579.72 | (Hex)₂ + (Man)₃(GlcNAc)₂ | 1.08 |

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. An isolated antibody or fragment thereof that binds to human intestinal alkaline phosphatase (hIAP) comprising:
a. a VH CDR1 comprising the amino acid sequence of SEQ ID NO: 3, a VH CDR2 comprising the amino acid sequence of SEQ ID NO: 4, a VH CDR3 comprising the amino acid sequence of SEQ ID NO: 5, or sequences at least 80% identical thereto, and/or
a VL CDR1 comprising the amino acid sequence of SEQ ID NO: 6, a VL CDR2 comprising the amino acid sequence of SEQ ID NO: 7, and a VL CDR3 comprising the amino acid sequence of SEQ ID NO: 8, or sequences at least 80% identical thereto; or
b. a VH CDR1 comprising the amino acid sequence of SEQ ID NO: 22, a VH CDR2 comprising the amino acid sequence of SEQ ID NO: 23 or SEQ ID NO: 31, a VH CDR3 comprising the amino acid sequence of SEQ ID NO: 24, or sequences at least 80% identical thereto, and/or
a VL CDR1 comprising the amino acid sequence of SEQ ID NO: 25, a VL CDR2 comprising the amino acid sequence of SEQ ID NO: 26, and a VL CDR3 comprising the amino acid sequence of SEQ ID NO: 27, or sequences at least 80% identical thereto.

2. An isolated antibody or fragment thereof that binds to human intestinal alkaline phosphatase (hIAP) comprising:
a. a heavy chain variable region comprising an amino acid sequence according to SEQ ID NO: 1 or a sequence that is at least 80% identical thereto, and/or a light chain variable region comprising an amino acid sequence according to SEQ ID NO: 2 or a sequence that is at least 80% identical thereto; or
b. a heavy chain variable region comprising an amino acid sequence according to SEQ ID NO: 20 or a sequence that is at least 80% identical thereto, and/or a light chain variable region comprising an amino acid sequence according to SEQ ID NO: 21 or a sequence that is at least 80% identical thereto.

3. The isolated monoclonal antibody or antigen-binding fragment of claim 1 or claim 2, wherein the isolated monoclonal antibody or antigen-binding fragment comprises a wildtype Fc or a modified Fc.

4. The isolated monoclonal antibody or antigen-binding fragment of claim 1 or claim 2, wherein the fragment comprises an Fab fragment, a single-chain variable fragment (scFv), or a single-domain antibody.

5. The isolated monoclonal antibody or antigen-binding fragment of claim 1 or claim 2, further comprising a detectable moiety.

6. A pharmaceutical composition comprising the isolated monoclonal antibody or fragment thereof according to claim 1 or claim 2 and a pharmaceutically acceptable carrier or excipient.

7. A nucleic acid encoding the antibody according to claim 1 or claim 2.

8. The nucleic acid of claim 7, wherein the nucleic acid comprises a nucleic acid sequence according to SEQ ID NO: 9 and/or SEQ ID NO: 10, SEQ ID NO: 28 and/or SEQ ID NO: 29, or a sequence that is at least 80% identical thereto.

9. A vector comprising the nucleic acid of claim 8.

10. An isolated cell comprising the vector of claim 9.

11. An isolated monoclonal antibody or fragment thereof that binds to human intestinal alkaline phosphatase (hIAP) comprising:
a. a heavy chain variable region encoded by a nucleic acid sequence according to SEQ ID NO: 9 or a sequence that is at least 80% identical thereto, and/or a light chain variable region encoded by a nucleic acid sequence according to SEQ ID NO: 10 or a sequence that is at least 80% identical thereto; or
b. a heavy chain variable region encoded by a nucleic acid sequence according to SEQ ID NO: 28 or a sequence that is at least 80% identical thereto, and/or a light chain variable region encoded by a nucleic acid sequence according to SEQ ID NO: 29 or a sequence that is at least 80% identical thereto.

12. An isolated cell comprising one or more polynucleotide(s) encoding the isolated monoclonal antibody or fragment thereof according to claim 1 or claim 2.

13. A method of detecting or diagnosing an iAP associated gastrointestinal disorder in a subject in need thereof, the method comprising administering to a subject a therapeutically effective amount of the hIAP antibody or fragment thereof of any one of claim 1, claim 2, or claim 4.

14. A method of treating or preventing an iAP associated gastrointestinal disease in a subject in need thereof, the method comprising administering to a subject a therapeutically effective amount of the hIAP antibody or fragment thereof of any one of claim 1, claim 2, or claim 4

15. The method of claim 13 or claim 14, wherein the iAP associated disease comprises a gastrointestinal disease.

16. The method of claim 15, wherein the gastrointestinal disease comprises colitis, inflammatory bowel disease (IBD), gastritis, gastroenteritis, pyloric stenosis, gastric cancer, gastrointestinal cancer, colon cancer, infectious diarrhea, fecal impaction, constipation, intestinal obstruction and pseudoobstruction, or malabsorption.

17. A kit comprising the isolated monoclonal antibody of claim 1 or claim 2.
